(19) United States
(12) Patent Application Publication
(10) Pub. No.: US 2022/0054544 A1
(43) Pub. Date: Feb. 24, 2022

(54) CONDITIONALLY ACTIVE RECEPTORS

(71) Applicant: Harpoon Therapeutics, Inc., South San Francisco, CA (US)

(72) Inventors: Shuoyen Jack LIN, San Bruno, CA (US); Richard J. AUSTIN, San Francisco, CA (US); Bryan D. LEMON, Mountain View, CA (US); Kevin WRIGHT, Redwood City, CA (US); Holger WESCHE, San Francisco, CA (US)

(21) Appl. No.: 17/276,777

(22) PCT Filed: May 14, 2019
(86) PCT No.: PCT/US19/32224

§ 371 Date: Mar. 16, 2021

Related U.S. Application Data
(60) Provisional application No. 62/734,952, filed on Sep. 21, 2018.

(51) Int. Cl. A61K 35/17; C07K 16/28; C07K 14/725

(52) U.S. Cl. CPC A61K 35/17; C07K 16/2818; C07K 2319/03; C07K 2317/565; C07K 14/7051

(57) ABSTRACT

Disclosed herein are conditionally active receptors which comprise a target antigen binding domain and a binding moiety. Each binding moiety comprises non-CDR loops for masking the binding of a target antigen binding domain to its target. The receptors are activated upon cleavage of the cleavable linkers. Pharmaceutical compositions comprising cells comprising the conditionally active receptors disclosed herein and methods of using such formulations are further provided.

### CROSS-REFERENCE

This application claims the benefit of U.S. Provisional Application No. 62/734,952, filed Sep. 21, 2018, which application is incorporated herein by reference in its entirety.

### SEQUENCE LISTING

The instant application contains a Sequence Listing which is hereby incorporated by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if set forth in their entireties.

### BACKGROUND OF THE INVENTION

The present disclosure provides conditionally active receptors which comprise a binding moiety comprising non-CDR loops, is activated upon cleavage, and can be used for diagnosing and treating cancers.

### SUMMARY OF THE INVENTION

One embodiment provides a conditionally active chimeric antigen receptor that comprises a single polypeptide chain, wherein the single polypeptide chain comprises (a) a binding moiety comprising a non-CDR loop and a cleavable linker; (b) a target antigen binding domain; (c) a transmembrane domain; and (d) an intracellular signaling domain; wherein the binding moiety is capable of masking the binding of the target antigen binding domain to its target.

In some embodiments, the binding moiety is a natural peptide, a synthetic peptide, or an engineered scaffold. In some embodiments, the engineered scaffold is a sdAb, a scFv, a Fab, a VHH, a fibronectin type III domain, immunoglobulin-like scaffold, DARPin, cystine knot peptide, lipocalin, three-helix bundle scaffold, protein G-related albumin-binding module, or a DNA or RNA aptamer scaffold. In some embodiments, the non-CDR-loop comprises a non-CDR loop of a variable domain, a constant domain, a C1-set domain, a C2-set domain, an I-domain, or any combinations thereof. In some embodiments, the binding moiety further comprises complementarity determining regions (CDRs). In some embodiments, the binding moiety is capable of binding an antigen, modifying a tumor microenvironment, activating an immune cell, or any combinations thereof. In some embodiments, the CDR loop provides a binding site specific for an antigen. In some embodiments, the antigen is a tumor antigen. In some embodiments, the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, the antigen comprises PD1 or CTLA4. In some embodiments, the binding moiety comprises a non-immunoglobulin molecule. In some embodiments, the non-immunoglobulin molecule comprises a natural peptide, a synthetic peptide, an engineered scaffold, or an engineered bulk serum protein. In some embodiments, the intracellular signaling domain comprises a signaling domain from ZAP70, CD3 zeta, CD28, or 4-1BB.

In some embodiments, the cleavable linker comprises a cleavage site. In some embodiments, the cleavage site is recognized by a protease, is pH sensitive, or is cleaved by chemical degradation. In some embodiments, the binding moiety is bound to the target antigen binding domain. In some embodiments, the binding moiety is covalently linked to the target antigen binding domain. In some embodiments, the binding moiety is capable of masking the binding of the target antigen binding domain to its target via specific intermolecular interactions between the binding moiety and the target antigen binding domain. In some embodiments, the non-CDR loop provides a binding site specific for binding of the moiety to the target antigen binding domain. In some embodiments, upon cleavage of the cleavage site, the binding moiety is separated from the target antigen binding domain and the target antigen binding domain binds to its target. In some embodiments, the target antigen binding domain binds to a tumor antigen. In some embodiments, the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT11. In some embodiments, the target antigen binding domain binds to an immune checkpoint protein. In some embodiments, the immune checkpoint protein comprises CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDOL IDO2, TDO, KIR, LAG-3, TIM-3, or VISTA. In some embodiments, the cleavage site is recognized by a serine protease, a cysteine protease, an aspartate protease, a threonine protease, a glutamic acid protease, a metalloproteinase, a gelatinase, or a asparagine peptide lyase. In some embodiments, the cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elatase, a cathepsin G, a proteinase 3, a neutrophil serine protease 4, a mast cell chymase, a mast cell tryptase, a dipeptidyl peptidase, or a dipeptidyl peptidase IV (DPPIV/CD26). In some embodiments, the target antigen binding domains comprises an sdAb, a scFv, a Fab, a variable heavy chain domain (VHH), or a combination thereof.

One embodiment provides a conditionally active T-cell receptor fusion protein that comprises a single polypeptide chain, comprising: (a) a T-cell receptor subunit comprising: (i) at least a portion of a T-cell receptor extracellular domain; (ii) a transmembrane domain; and (iii) a T-cell receptor intracellular domain comprising a stimulatory domain from an intracellular signaling domain; (b) a binding moiety comprising a non-CDR loop and a cleavable linker; and (c) a target antigen binding domain; wherein the binding moiety is capable of masking the binding of the target antigen binding domain to its target. In some embodiments, the conditionally active T-cell receptor fusion protein of further comprises a costimulatory domain, wherein the costimulatory domain is a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

In some embodiments, the at least one but not more than 20 modifications thereto comprises a modification of an amino acid that mediates cell signaling or a modification of an amino acid that is phosphorylated in response to a ligand binding to the encoded T-cell receptor fusion protein. In some embodiments, the encoded transmembrane domain comprises a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CDS, CD8, CD9, CD16, CD22, CD33, CD28, CD37, CD64, CD80, CD86, CD134, CD137, CD154, functional fragments thereof, and amino acid sequences thereof having at least one but not more than 20 modifications thereto. In some embodiments, the T-cell receptor fusion comprises an immunoreceptor tyrosine-based activation motif (ITAM) or portion thereof, wherein the ITAM or portion thereof is from a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b 1 chain, Fc gamma receptor 2b2 chain, Fc gamma receptor 3a chain, Fc gamma receptor 3b chain, Fc beta receptor 1 chain, TYROBP (DAP12), CDS, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some embodiments, the T-cell receptor intracellular domain is derived from CD3 epsilon CD3 gamma, CD3 delta, CD3 alpha, CD3 beta, or a combination thereof. In some embodiments, the T-cell receptor subunit and the target antigen binding domain are operatively linked. In some embodiments, the T-cell receptor fusion protein incorporates into a T-cell receptor when expressed in a T-cell. In some embodiments, the antigen binding domain comprises a domain from a human or humanized antibody. In some embodiments, the binding moiety comprises a natural peptide, a synthetic peptide, or an engineered scaffold. In some embodiments, the engineered scaffold comprises a sdAb, a scFv, a Fab, a VHH, a fibronectin type III domain, an immunoglobulin-like scaffold, a DARPin, a cystine knot peptide, a lipocalin, or a three-helix bundle scaffold. In some embodiments, the non-CDR-loop comprises a non-CDR loop of a variable domain, a constant domain, a C1-set domain, a C2-set domain, an I-domain, or any combinations thereof. In some embodiments, the binding moiety further comprises complementarity determining regions (CDRs). In some embodiments, the binding moiety is capable of binding an antigen, modifying a tumor microenvironment, activating an immune cell, or any combinations thereof. In some embodiments, the CDR loop provides a binding site specific for an antigen. In some embodiments, the antigen comprises a tumor antigen.

In some embodiments, the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, the antigen comprises PD1 or CTLA4. In some embodiments, the intracellular signaling domain comprises a signaling domain from ZAP70, CD3 zeta, CD28, or 4-1BB. In some embodiments, the cleavable linker comprises a cleavage site. In some embodiments, the cleavage site is recognized by a protease, is pH sensitive, or is cleaved by chemical degradation. In some embodiments, the binding moiety is bound to the target antigen binding domain. In some embodiments, the binding moiety is covalently linked to the target antigen binding domain. In some embodiments, the binding moiety is capable of masking the binding of the target antigen binding domain to its target via specific intermolecular interactions between the binding moiety and the target antigen binding domain. In some embodiments, the non-CDR loop provides a binding site specific for binding of the moiety to the antigen binding domain. In some embodiments, upon cleavage of the cleavage site, the binding moiety is separated from the target antigen binding domain and the target antigen binding domain binds to its target. In some embodiments, the target antigen binding domain binds to a tumor antigen. In some embodiments, the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, the target antigen binding domain binds to an immune checkpoint protein. In some embodiments, the immune checkpoint protein is CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA. In some embodiments, the cleavage site is recognized by a serine protease, a cysteine protease, an aspartate protease, a threonine protease, a glutamic acid protease, a metalloproteinase, a gelatinase, or a asparagine peptide lyase. In some embodiments, the cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elatase, a cathepsin G, a proteinase 3, a neutrophil serine protease 4, a mast cell chymase, a mast cell tryptase, a dipeptidyl peptidase, or a dipeptidyl peptidase IV (DPPIV/CD26). In some embodiments, the target antigen binding domains comprises an sdAb, a scFv, a Fab, a variable heavy chain domain (VHH), or a combination thereof.

One embodiment provides a conditionally active T-cell receptor comprising a binding moiety comprising a non-CDR loop and a cleavable linker, wherein the binding moiety is capable of masking the binding of the T-cell receptor to its target. In some embodiments, the non-CDR loop of the binding moiety provides a binding site specific for T-cell receptor alpha, T-cell receptor beta, or a combination thereof. In some embodiments, the binding moiety is a natural peptide or, a synthetic peptide, an engineered scaffold. In some embodiments, the engineered scaffold comprises a sdAb, a scFv, a Fab, a VHH, a fibronectin type III domain, an immunoglobulin-like scaffold, a DARPin, a cystine knot peptide, a lipocalin, or a three-helix bundle scaffold. In some embodiments, the non-CDR-loop comprises a non-CDR loop of a variable domain, a constant domain, a C1-set domain, a C2-set domain, an I-domain, or any combinations thereof. In some embodiments, the binding moiety further comprises complementarity determining regions (CDRs). In some embodiments, the binding moiety is capable of binding an antigen, modifying a tumor microenvironment, activating an immune cell, or any combinations thereof. In some embodiments, the CDR loop provides a binding site specific for an antigen. In some embodiments, the antigen comprises a tumor antigen. In some embodiments, the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, the antigen comprises PD1 or CTLA4.

In some embodiments, the intracellular signaling domain comprises a signaling domain from ZAP70, CD3 zeta, CD28, or 4-1BB. In some embodiments, the cleavable linker comprises a cleavage site. In some embodiments, the cleavage site is recognized by a protease, is pH sensitive, or is cleaved by chemical degradation. In some embodiments, the binding moiety is bound to the target antigen binding domain. In some embodiments, the binding moiety is covalently linked to the target antigen binding domain. In some embodiments, the binding moiety is capable of masking the binding of the target antigen binding domain to its target via specific intermolecular interactions between the binding moiety and the target antigen binding domain. In some embodiments, the non-CDR loop provides a binding site specific for binding of the moiety to the target antigen binding domain. In some embodiments, upon cleavage of the cleavage site, the binding moiety is separated from the target antigen binding domain and the target antigen binding domain binds to its target. In some embodiments, the target antigen binding domain binds to a tumor antigen. In some embodiments, the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, the target antigen binding domain binds to an immune checkpoint protein. In some embodiments, the immune checkpoint protein comprises CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA. In some embodiments, the protease cleavage site is recognized by a serine protease, a cysteine protease, an aspartate protease, a threonine protease, a glutamic acid protease, a metalloproteinase, a gelatinase, or a asparagine peptide lyase. In some embodiments, the protease cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elatase, a cathepsin G, a proteinase 3, a neutrophil serine protease 4, a mast cell chymase, a mast cell tryptase, a dipeptidyl peptidase, or a dipeptidyl peptidase IV (DPPIV/CD26). In some embodiments, the target antigen binding domains comprises an sdAb, a scFv, a Fab, a variable heavy chain domain (VHH), or a combination thereof.

In one aspect, the present disclosure provides a conditionally active chimeric antigen receptor comprising a single polypeptide chain, comprising (a) a binding moiety comprising a non-CDR loop and a cleavable linker; (b) a target antigen binding domain; (c) a transmembrane domain; and (d) an intracellular signaling domain; wherein the binding moiety is capable of masking the binding of the target antigen binding domain to its target. In some embodiments, the binding moiety is a natural peptide, a synthetic peptide, or an engineered scaffold. In some embodiments, the engineered scaffold is a sdAb, a scFv, a Fab, a VHH, a fibronectin type III domain, immunoglobulin-like scaffold, DARPin, cystine knot peptide, lipocalin, three-helix bundle scaffold, protein G-related albumin-binding module, or a DNA or RNA aptamer scaffold. In some embodiments, the non-CDR-loop comprises a non-CDR loop of a variable domain, a constant domain, a C1-set domain, a C2-set domain, an I-domain, or any combinations thereof. In some embodiments, the binding moiety further comprises complementarity determining regions (CDRs). In some embodiments, the binding moiety is capable of binding a target antigen binding domain, modifying a tumor microenvironment, activating an immune cell, or any combinations thereof. In some embodiments, the CDR loop provides a binding site specific for an antigen. In some embodiments, the antigen is a tumor antigen. In some embodiments, the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, the antigen comprises PD1 or CTLA4. In some embodiments, the intracellular signaling domain comprises a signaling domain from ZAP70, CD3 zeta, CD28, or 4-1BB. In some embodiments, the cleavable linker comprises a cleavage site. In some embodiments, the cleavage site is recognized by a protease, is pH sensitive, or is cleaved by chemical degradation. In some embodiments, the binding moiety is bound to the target antigen binding domain. In some embodiments, the binding moiety is covalently linked to the target antigen binding domain. In some embodiments, the binding moiety is capable of masking the binding of the target antigen binding domain to its target via specific intermolecular interactions between the binding moiety and the target antigen binding domain. In some embodiments, the non-CDR loop provides a binding site specific for binding of the moiety to the target antigen binding domain. In some embodiments, upon cleavage of the cleavage site, the binding moiety is separated from the target antigen binding domain and the target antigen binding domain binds to its target. In some embodiments, the target antigen binding domain binds to a tumor antigen. In some embodiments, the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, the target antigen binding domain binds to an immune checkpoint protein. In some embodiments, the immune checkpoint protein comprises CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDOL IDO2, TDO, KIR, LAG-3, TIM-3, or VISTA. In some embodiments, the cleavage site is recognized by a serine protease, a cysteine protease, an aspartate protease, a threonine protease, a glutamic acid protease, a metalloproteinase, a gelatinase, or a asparagine peptide lyase. In some embodiments, the cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elatase, a cathepsin G, a proteinase 3, a neutrophil serine protease 4, a mast cell chymase, a mast cell tryptase, a dipeptidyl peptidase, or a dipeptidyl peptidase IV (DPPIV/CD26). In some embodiments, the target antigen binding domains comprises an sdAb, a scFv, a Fab, a variable heavy chain domain (VHH), or a combination thereof.

In another aspect, the present disclosure provides a conditionally active T-cell receptor fusion protein comprising a single polypeptide chain comprising: (a) a T-cell receptor subunit comprising (i) at least a portion of a T-cell receptor extracellular domain; (ii) a transmembrane domain; and (iii) a T-cell receptor intracellular domain comprising a stimulatory domain from an intracellular signaling domain; (b) a binding moiety comprising a non-CDR loop and a cleavable linker; and (c) a target antigen binding domain; wherein the binding moiety is capable of masking the binding of the target antigen binding domain to its target. In some embodiments, the T-cell receptor fusion protein further comprises a costimulatory domain, wherein the costimulatory domain is a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto. In some embodiments, the at least one but not more than 20 modifications thereto comprises a modification of an amino acid that mediates cell signaling or a modification of an amino acid that is phosphorylated in response to a ligand binding to the encoded T-cell receptor fusion protein. In some embodiments, the encoded transmembrane domain comprises a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CDS, CD8, CD9, CD16, CD22, CD33, CD28, CD37, CD64, CD80, CD86, CD134, CD137, CD154, functional fragments thereof, and amino acid sequences thereof having at least one but not more than 20 modifications thereto. In some embodiments, the T-cell receptor fusion comprises an immunoreceptor tyrosine-based activation motif (ITAM) or portion thereof, wherein the ITAM or portion thereof is from a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b 1 chain, Fc gamma receptor 2b2 chain, Fc gamma receptor 3a chain, Fc gamma receptor 3b chain, Fc beta receptor 1 chain, TYROBP (DAP12), CDS, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some embodiments, the T-cell receptor intracellular domain is derived from CD3 epsilon CD3 gamma, CD3 delta, CD3 alpha, CD3 beta, or a combination thereof. In some embodiments, the T-cell receptor subunit and the target antigen binding domain are operatively linked. In some embodiments, the T-cell receptor fusion protein incorporates into a T-cell receptor when expressed in a T-cell. In some embodiments, the antigen binding domain comprises a domain from a human or humanized antibody. In some embodiments, the binding moiety comprises a natural peptide, a synthetic peptide, or an engineered scaffold. In some embodiments, the engineered scaffold comprises a sdAb, a scFv, a Fab, a VHH, a fibronectin type III domain, an immunoglobulin-like scaffold, a DARPin, a cystine knot peptide, a lipocalin, or a three-helix bundle scaffold. In some embodiments, the non-CDR-loop comprises a non-CDR loop of a variable domain, a constant domain, a C1-set domain, a C2-set domain, an I-domain, or any combinations thereof. In some embodiments, the binding moiety further comprises complementarity determining regions (CDRs). In some embodiments, the binding moiety is capable of binding an antigen, modifying a tumor microenvironment, activating an immune cell, or any combinations thereof. In some embodiments, the CDR loop provides a binding site specific for an antigen. In some embodiments, the antigen comprises a tumor antigen. In some embodiments, the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, the antigen comprises PD1 or CTLA4. some embodiments, the intracellular signaling domain comprises a signaling domain from ZAP70, CD3 zeta, CD28, or 4-1BB. In some embodiments, the cleavable linker comprises a cleavage site. In some embodiments, the cleavage site is recognized by a protease, is pH sensitive, or is cleaved by chemical degradation. In some embodiments, the binding moiety is bound to the target antigen binding domain. In some embodiments, the binding moiety is covalently linked to the target antigen binding domain. In some embodiments, the binding moiety is capable of masking the binding of the target antigen binding domain to its target via specific intermolecular interactions between the binding moiety and the target antigen binding domain. In some embodiments, the non-CDR loop provides a binding site specific for binding of the moiety to the target antigen binding domain. In some embodiments, upon cleavage of the cleavage site, the binding moiety is separated from the target antigen binding domain and the target antigen binding domain binds to its target. In some embodiments, the target antigen binding domain binds to a tumor antigen. In some embodiments, the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, the target antigen binding domain binds to an immune checkpoint protein. In some embodiments, the immune checkpoint protein is CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA. In some embodiments, the cleavage site is recognized by a serine protease, a cysteine protease, an aspartate protease, a threonine protease, a glutamic acid protease, a metalloproteinase, a gelatinase, or a asparagine peptide lyase. In some embodiments, the cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elatase, a cathepsin G, a proteinase 3, a neutrophil serine protease 4, a mast cell chymase, a mast cell tryptase, a dipeptidyl peptidase, or a dipeptidyl peptidase IV (DPPIV/CD26). In some embodiments, the target antigen binding domains comprises an sdAb, a scFv, a Fab, a variable heavy chain domain (VHH), or a combination thereof.

In another aspect, the present disclosure provides a conditionally active T-cell receptor comprising a binding moiety comprising a non-CDR loop and a cleavable linker, wherein the binding moiety is capable of masking the binding of the T-cell receptor to its target. In some embodiments, the non-CDR loop of the binding moiety provides a binding site specific for T-cell receptor alpha, T-cell receptor beta, or a combination thereof. In some embodiments, the binding moiety is a natural peptide or, a synthetic peptide, an engineered scaffold. In some embodiments, the engineered scaffold comprises a sdAb, a scFv, a Fab, a VHH, a fibronectin type III domain, an immunoglobulin-like scaffold, a DARPin, a cystine knot peptide, a lipocalin, or a three-helix bundle scaffold. In some embodiments, the non-CDR-loop comprises a non-CDR loop of a variable domain, a constant domain, a C1-set domain, a C2-set domain, an I-domain, or any combinations thereof. In some embodiments, the binding moiety further comprises complementarity determining regions (CDRs). In some embodiments, the binding moiety is capable of binding a target antigen binding domain, modifying a tumor microenvironment, activating an immune cell, or any combinations thereof. In some embodiments, the CDR loop provides a binding site specific for an antigen. In some embodiments, the antigen comprises a tumor antigen. In some embodiments, the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, the cleavable linker comprises a cleavage site. In some embodiments, the cleavage site is recognized by a protease, is pH sensitive, or is cleaved by chemical degradation. In some embodiments, the binding moiety is bound to the target antigen binding domain. In some embodiments, the binding moiety is covalently linked to the target antigen binding domain. In some embodiments, the binding moiety is capable of masking the binding of the target antigen binding domain to its target via specific intermolecular interactions between the binding moiety and the target antigen binding domain. In some embodiments, the non-CDR loop provides a binding site specific for binding of the moiety to the target antigen binding domain. In some embodiments, upon cleavage of the cleavage site, the binding moiety is separated from the target antigen binding domain and the target antigen binding domain binds to its target. In some embodiments, the target antigen binding domain binds to a tumor antigen. In some embodiments, the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, the target antigen binding domain binds to an immune checkpoint protein. In some embodiments, the immune checkpoint protein comprises CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA. In some embodiments, the protease cleavage site is recognized by a serine protease, a cysteine protease, an aspartate protease, a threonine protease, a glutamic acid protease, a metalloproteinase, a gelatinase, or a asparagine peptide lyase. In some embodiments, the protease cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elatase, a cathepsin G, a proteinase 3, a neutrophil serine protease 4, a mast cell chymase, a mast cell tryptase, a dipeptidyl peptidase, or a dipeptidyl peptidase IV (DPPIV/CD26). In some embodiments, the target antigen binding domains comprises an sdAb, a scFv, a Fab, a variable heavy chain domain (VHH), or a combination thereof. In some embodiments, the binding moiety is capable of aiding the expansion of the masked proCAR in patients. In some cases, this circumvents problems associated with lack of antigen-dependent expansion, as experienced by other CARs.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a variable domain of an immunoglobulin molecule, comprising complementarity determining regions (CDR1, CDR2, and CDR3), and non-CDR loops connecting the beta strand (AB, CC′, C″ D, EF, and DE).

FIG. 2 illustrates an exemplary arrangement of a target antigen binding domain (aTarget 1), cleavable linker, and a binding moiety (aTarget 2) of the present disclosure.

FIG. 3 illustrates an example of a conditionally active receptor as described herein.

FIGS. 4A-E show examples of receptor constructs which were generated as disclosed herein used in Examples 12-16. Each construct comprises an intracellular domain comprising a CD8 hinge/transmembrane domain (SEQ ID NO: 62), a 4-1BB intracellular domain (SEQ ID NO: 63), and a CD3 zeta intracellular domain (SEQ ID NO: 64). Construct C1081 (SEQ ID NO: 43; FIG. 4A) does not comprise a target antigen binding domain or a binding moiety. Construct C1824 (SEQ ID NO: 44; FIG. 4B) comprises an anti-EGFR antigen binding domain EH4 (SEQ ID NO: 60) but does not comprise a binding moiety. Construct 1826 (SEQ ID NO: 45; FIG. 4C) comprises target antigen binding domain EH4 and the binding moiety 10G with an unmodified non-CDR loop (SEQ ID NO: 56) along with a non-cleavable linker (SEQ ID NO: 65). Construct 1950 (SEQ ID NO: 46; FIG. 4D) comprises target antigen binding domain EH4 and binding moiety 10G with a modified non-CDR loop (SEQ ID NO: 57) along with a non-cleavable linker (SEQ ID NO: 65). Construct 2031 (SEQ ID NO: 47; FIG. 4E) comprises target antigen binding domain EH4 and binding moiety 10G with a modified non-CDR loop (SEQ ID NO: 57) along with protease cleavable linker 1 (SEQ ID NO: 66).

FIG. 5 shows results from experiments described in Example 12.

FIG. 6 shows results from experiments described in Example 13.

FIGS. 7A-C show results from experiments described in Example 14 for low (FIG. 7A), medium (FIG. 7B), and high (FIG. 7C) CAR expression based on anti-FLAG staining.

FIG. 8 shows results from experiments described in Example 15.

FIG. 9 shows results from experiments in Example 16.

FIGS. 10A-H show examples of receptor constructs which were generated as disclosed herein. Construct C1081 (SEQ ID NO: 43; FIG. 10A) does not comprise a target antigen binding domain or a binding moiety. Construct 1827 (SEQ ID NO: 48; FIG. 10B) comprises an anti-EGFR antigen-binding domain EH104 (SEQ ID NO: 61) but does not comprise a binding moiety. Construct 1829 (SEQ ID NO: 49; FIG. 10C) comprises target antigen binding domain EH104 and the binding moiety 10G with an unmodified non-CDR loop (SEQ ID NO: 56) along with a non-cleavable linker (SEQ ID NO: 65). Construct 1952 (SEQ ID NO: 50; FIG. 10D) comprises target antigen binding domain EH104 and binding moiety 10G with a modified non-CDR loop (SEQ ID NO: 58) along with a non-cleavable linker (SEQ ID NO: 65). Construct 1954 (SEQ ID NO: 51; FIG. 10E) comprises target antigen binding domain EH104 and binding moiety 10G with a modified non-CDR loop (SEQ ID NO: 59) along with a non-cleavable linker (SEQ ID NO: 65). Construct 2033 (SEQ ID NO: 52; FIG. 10F) comprises target antigen binding domain EH104 and binding moiety 10G with a modified non-CDR loop (SEQ ID NO: 58) along with protease cleavable linker 1 (SEQ ID NO: 66). Construct 1953 (SEQ ID NO: 53; FIG. 10G) comprises target antigen binding domain EH104 and binding moiety 10G with a modified non-CDR loop (SEQ ID NO: 59) along with protease cleavable linker 2 (SEQ ID NO: 67). Construct 2035 (SEQ ID NO: 54; FIG. 10H) comprises target antigen binding domain EH104 and binding moiety 10G with a modified non-CDR loop (SEQ ID NO: 59) along with protease cleavable linker 1 (SEQ ID NO: 66).

FIG. 11 shows results from experiments described in Example 17.

FIG. 12 shows results from experiments described in Example 18.

FIGS. 13A-C show results from experiments described in Example 19 for low (FIG. 13A), medium (FIG. 13B), and high (FIG. 13C) CAR expression based on anti-FLAG staining.

FIGS. 14A-C show results from experiments described in Example 20 for low (FIG. 14A), medium (FIG. 14B), and high (FIG. 14C) CAR expression based on anti-FLAG staining.

FIG. 15 shows results from experiments described in Example 21.

FIG. 16 shows results from experiments described in Example 22.

FIG. 17 shows results from experiments described in Example 23.

FIGS. 18A-E illustrate exemplary ProCAR constructs. FIG. 18A illustrates construct C2446 which includes an anti-human serine albumin sdAb, a protease cleavage site 3, an anti-human EGFR sdAb, a FLAG epitope, a CD8 hinge/transmembrane domain, a 4-1BB intracellular domain, and a CD3 zeta intracellular domain (SEQ ID NO: 71). FIG. 18B illustrates construct C2510 which includes an anti-human serine albumin sdAb, a protease cleavage site, an anti-human EGFR sdAb, a FLAG epitope, a dimerization-deficient CD8a hinge/transmembrane domain, a 4-1BB intracellular domain, and a CD3 zeta intracellular domain (SEQ ID NO: 73). FIG. 18C illustrates construct C2513 which includes an anti-human serine albumin sdAb, a protease cleavage site 3, an anti-human EGFR sdAb, a FLAG epitope, a dimerization-deficient CD8a hinge/transmembrane domain, a 4-1BB intracellular domain, and a CD3 zeta intracellular domain (SEQ ID NO: 74). FIG. 18D illustrates construct C2514 which includes an anti-human serine albumin sdAb, an anti-human EGFR sdAb, a FLAG epitope, a dimerization-deficient CD8a hinge/transmembrane domain, a 4-1BB intracellular domain, and a CD3 zeta intracellular domain (SEQ ID NO: 75). FIG. 18E illustrates construct C2448, which includes an anti-human EGFR sdAb, a FLAG epitope, a dimerization-deficient CD8a hinge/transmembrane domain, a 4-1BB intracellular domain, and a CD3 zeta intracellular domain (SEQ ID NO: 72).

FIGS. 19A-B illustrate that the level of protease site-dependent cell killing activity of the ProCARs constructs. The level of protease site-dependent cell killing activity of the ProCARs constructs was reduced by eliminating the dimerization activity of the CD8a transmembrane domain (FIG. 19B) compared to wild-type (FIG. 19A).

FIG. 20 illustrates that the dimerization deficient EGFR CAR ad similar cell killing activity compared to wild-type.

FIGS. 21A-G illustrate exemplary ProCAR constructs. FIG. 21A illustrates construct C2483 which includes an anti-human EpCAM sdAb, a FLAG epitope, a CD8 hinge/transmembrane domain, a 4-1BB intracellular domain, and a CD3 zeta intracellular domain (SEQ ID NO: 77). FIG. 21B illustrates construct C2790 which includes an anti-human serum albumin sdAb, an anti-human EpCAM sdAb, a FLAG epitope, a CD8 hinge/transmembrane domain, a 4-1BB intracellular domain, and a CD3 zeta intracellular domain (SEQ ID NO: 78). FIG. 21C illustrates construct C3269 which includes an anti-human serum albumin sdAb that contains a mask in the CC′ loop, an anti-human EpCAM sdAb, a FLAG epitope, a CD8 hinge/transmembrane domain, a 4-1BB intracellular domain, and a CD3 zeta intracellular domain (SEQ ID NO: 79). FIG. 21D illustrates construct C3272 which includes an anti-human serum albumin sdAb that contains a mask in the CC′ loop, an anti-human EpCAM sdAb, a FLAG epitope, a CD8 hinge/transmembrane domain, a 4-1BB intracellular domain, and a CD3 zeta intracellular domain (SEQ ID NO: 80). FIG. 21E illustrates construct C3329 which includes an anti-human serum albumin sdAb, a protease cleavage site 3, an anti-human EpCAM sdAb, a FLAG epitope, a CD8 hinge/transmembrane domain, a 4-1BB intracellular domain, and a CD3 zeta intracellular domain (SEQ ID NO: 82). FIG. 21F illustrates construct C3328 which includes an anti-human serum albumin sdAb that contains a mask in the CC′ loop, a protease cleavage site 3, an anti-human EpCAM sdAb, a FLAG epitope, a CD8 hinge/transmembrane domain, a 4-1BB intracellular domain, and a CD3 zeta intracellular domain (SEQ ID NO: 81). FIG. 21G illustrates construct C2780 which includes an anti-GFP sdAb, a FLAG epitope, a CD8 hinge/transmembrane domain, a 4-1BB intracellular domain, and a CD3 zeta intracellular domain (SEQ ID NO: 76).

FIG. 22 demonstrates steric blocking of anti-EpCAM sdAb H90 ProCAR-T cell killing activity by HSA when assayed at ratios 10:1, 5:1, 2.5:1, and 1.25:1 CAR-T:Target cells.

FIGS. 23A-C provide histograms of EpCAM-Fc/Alexa Fluor 647 staining of CAR-T cells from FIG. 21 that have been grouped into low (FIG. 23A), medium (FIG. 23B), or high (FIG. 23C) CAR expression based on anti-FLAG staining that demonstrate the efficacy of EpCAM mask 1 in blocking ProCAR EpCAM-binding activity.

FIGS. 24A-C provide histograms of EpCAM-Fc/Alexa Fluor 647 staining of CAR-T cells from FIG. 21 that have been grouped into low (FIG. 24A), medium (FIG. 24B), or high (FIG. 24C) CAR expression based on anti-FLAG staining that demonstrate the efficacy of EpCAM mask 2 in blocking ProCAR EpCAM-binding activity.

FIG. 25 demonstrates masking of the anti-EpCAM sdAb H90 of constructs of FIG. 21 at ratios 10:1, 5:1, 2.5:1, and 1.25:1 CAR-T:Target cells.

FIG. 26 demonstrates protease site-dependent activation of EpCAM ProCAR mask 2 cell killing activity.

FIGS. 27A-C illustrate protease activation of EpCAM Mask 1 ProCAR antigen binding activity at low (FIG. 27A), medium (FIG. 27B), or high (FIG. 27C) CAR expression based on anti-FLAG staining.

FIGS. 28A-C illustrate protease activation of EpCAM Mask 2 ProCAR antigen binding activity at low (FIG. 28A), medium (FIG. 28B), or high (FIG. 28C) CAR expression based on anti-FLAG.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

A “single chain Fv” or “scFv,” as used herein, refers to a binding protein in which the variable domains of the heavy chain and of the light chain of a traditional two chain antibody are joined to form one chain. Typically, a linker peptide is inserted between the two chains to allow for proper folding and creation of an active binding site.

A “cleavage site for a protease,” or “protease cleavage site”, as meant herein, is an amino acid sequence that can be cleaved by a protease, such as, for example, a matrix metalloproteinase or a furin. Examples of such sites include Gly-Pro-Leu-Gly-Ile-Ala-Gly-Gln (SEQ ID NO: 91) or Ala-Val-Arg-Trp-Leu-Leu-Thr-Ala (SEQ ID NO: 92), which can be cleaved by metalloproteinases, and Arg-Arg-Arg-Arg-Arg-Arg (SEQ ID NO: 93), which is cleaved by a furin. In therapeutic applications, the protease cleavage site can be cleaved by a protease that is produced by target cells, for example cancer cells or infected cells, or pathogens.

As used herein, “non-CDR loops” within immunoglobulin (Ig) molecules are regions of a polypeptide other than the complimentarity determining regions (CDRs) of an antibody. These regions may be derived from an antibody or an antibody fragment. These regions may also be synthetically or artificially derived, such as through mutagenesis or polypeptide synthesis.

In an Ig, Ig-like, or beta-sandwich scaffold that has 9 beta-strands (e.g., a VH, a VL, a camelid VHH, a sdAb), the non-CDR loops can refer to the AB, CC′, C″D, EF loops or loops connecting beta-strands proximal to the C-terminus. In an Ig, Ig-like, or beta-sandwich scaffold that has 7 beta-strands (e.g., a CH, a CL, an adnectin, a Fn-III), the non-CDR loops can refer to the AB, CD, and EF loops or loops connecting beta-strands proximal the C-terminus. In other Ig-like or beta-sandwich scaffolds, the non-CDR loops are the loops connecting beta-strands proximal to the C-terminus or topologically equivalent residues using the framework established in the Halaby 1999 publication (Prot Eng Des Sel., 12:563-571).

In a non-beta-sandwich scaffold (e.g., a DARPin, an affimer, an affibody), the “non-CDR loops” refer to an area that is (1) amenable for sequence randomization to allow engineered specificities to a second antigen, and (2) distal to the primary specificity determining region(s) typically used on the scaffold to allow simultaneous engagement of the scaffold to both antigens without steric interference. For this purpose, the primary specificity determining region(s) can be defined using the framework established in the Skrlec 2015 publication (Trends in Biotechnol, 33:408-418). An excerpt of the framework is listed below:

| Scaffold | Primary specificity determining region(s) |
|---|---|
| Affibody | 13 residues in two helices |
| Affimer | 12-36 residues |
| Anticalin | Four loops (up to 24 aa) |
| Avimer | 11 residues |
| Centyrin | 13 residues |
| DARPin | 7 residues in each n-repeat, or |
| | 8 residues in each n-repeat |
| Fynomer | 6 residues in the RT- and n-Src-loop |
| Kunitz domain | 1-2 loops |

“Chimeric antigen receptor” or “CAR” or “CARs”, as used herein, refers to engineered receptors which provide antigen specificity to cells (for example T cells). CARs comprise multiple domains, for example, at least one target antigen binding domain, a transmembrane domain, one or more co-stimulatory domains, and an intracellular signaling domain. Each domain may be connected by a linker.

“Target antigen binding domain”, as used herein, refers to a region which targets a specific antigen. A target antigen binding domain comprises, for example an sdAb, a scFv, a variable heavy domain (VHH), a full length antibody, or any other peptide that has a binding affinity towards a specific antigen. The target antigen binding domain does not include a cytokine.

A “cytokine,” as meant herein, refers to intercellular signaling molecules, and active fragments and portions thereof, which are involved in the regulation of mammalian somatic cells. A number of families of cytokines, for examples, interleukins, interferons, and transforming growth factors are included.

“Intracellular signaling domain” or “cytoplasmic domain”, as used herein, refer to a region of a receptor which transduces the effector function signal.

“Co-stimulatory domain”, as used herein, refers to a region of a receptor which enhances proliferation, survival, and/or development of the cell. Examples include members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 and combinations thereof.

“Transmembrane domain”, as used herein, refers to the region of a receptor which crosses the plasma membrane. Examples include the transmembrane region of a transmembrane protein (for example a Type 1 transmembrane protein), an artificial hydrophobic sequence, and a combination thereof.

“Immune cell”, as used herein, refers to a cell of the mammalian immune system, including but not limited to antigen presenting cells, B-cells, basophils, cytotoxic T-cells, dendritic cells, eosinophils, granulocytes, helper T-cells, leukocytes, lymphocytes, macrophages, mast cells, memory cells, monocytes, natural killer cells, neutrophils, phagocytes, plasma cells and T-cells.

### Binding Moiety for Masking of Target Antigen Binding Domains

The conditionally active receptors described herein comprise at least one binding moiety comprising a non-CDR loop. In one aspect, the binding moiety masks binding of a target antigen binding domain to a target antigen until activation. The cleavable linker, for example, comprises a protease cleavage site or a pH dependent cleavage site. The cleavable linker, in certain instances, is cleaved only in a tumor microenvironment. Thus, the binding moiety, connected to the cleavable linker, and further bound to the target antigen binding domain, in some examples, maintains the target antigen binding domain in an inert state in circulation until the cleavable linker is cleaved off in a tumor microenvironment. In some embodiments, the binding moiety binds to the target antigen binding domain. In some embodiments, a non-CDR loop provides a binding site for binding of the moiety to the target antigen binding domain. In some embodiments, the binding moiety masks binding of the target binding domain to the target antigen, e.g. via steric occlusion, via specific intramolecular interactions, such as interactions within the different domains of the polypeptide comprising the binding moiety. In some embodiments, the binding moiety further comprises complimentary determining regions (CDRs).

In some instances, the binding moiety is a domain derived from an immunoglobulin molecule (Ig molecule). The Ig may be of any class or subclass (IgG1, IgG2, IgG3, IgG4, IgA, IgE, IgM etc). A polypeptide chain of an Ig molecule folds into a series of parallel beta strands linked by loops. In the variable region, three of the loops constitute the “complementarity determining regions” (CDRs) which determine the antigen binding specificity of the molecule. An IgG molecule comprises at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs) with are hypervariable in sequence and/or involved in antigen recognition and/or usually form structurally defined loops, interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments of this disclosure, at least some or all of the amino acid sequences of FR1, FR2, FR3, and FR4 are part of the “non-CDR loop” of the binding moiety described herein. As shown in FIG. 1, a variable domain of an immunoglobulin molecule has several beta strands that are arranged in two sheets. The variable domains of both light and heavy immunoglobulin chains contain three hypervariable loops, or complementarity-determining regions (CDRs). The three CDRs of a V domain (CDR1, CDR2, CDR3) cluster at one end of the beta barrel. The CDRs are the loops that connect beta strands B-C, C′-C″, and F-G of the immunoglobulin fold, whereas the bottom loops that connect beta strands AB, CC′, C″ D and EF of the immunoglobulin fold, and the top loop that connects the D-E strands of the immunoglobulin fold are the non-CDR loops.

Within the variable domain, the CDRs are believed to be responsible for antigen recognition and binding, while the FR residues are considered a scaffold for the CDRs. However, in certain cases, some of the FR residues play an important role in antigen recognition and binding. Framework region residues that affect Ag binding are divided into two categories. The first are FR residues that contact the antigen, thus are part of the binding-site, and some of these residues are close in sequence to the CDRs. Other residues are those that are far from the CDRs in sequence, but are in close proximity to it in the 3-D structure of the molecule, e.g., a loop in heavy chain.

The binding moiety may be any kind of polypeptide. In some embodiments, the binding moiety is a natural peptide, a synthetic peptide, or an engineered serum bulk protein. In some embodiments, the binding moiety is an engineered scaffold. The engineered scaffold comprises, for example, sdAb, a scFv, a Fab, a VHH, a fibronectin type III domain, immunoglobulin-like scaffold (as suggested in Halaby et al., 1999. Prot. Eng. 12(7):563-571), DARPin, cystine knot peptide, lipocalin, three-helix bundle scaffold, protein G-related albumin-binding module, or a DNA or RNA aptamer scaffold.

The non-CDR loop may be any region that is not a complimentary determining region (CDR) of an antibody. In some embodiments, the non-CDR loop is derived from an antibody or a fibronectin. In some embodiments, the non-CDR loop is artificially or synthetically generated, for example, by mutagenesis or polypeptide synthesis.

In some embodiments, the binding moiety performs a secondary function in addition to inhibiting binding of a target antigen binding domain to a target antigen. In some embodiments, the secondary function is performed prior to cleavage of a cleavable linker. In some embodiments, the secondary function is performed after cleavage of a cleavable linker. In other embodiments, the secondary function is performed both before and after cleavage of a cleavable linker. The secondary function may include, but is not limited to, antigen binding, microenvironment modification, immune cell activation or a combination thereof.

In some embodiments, the binding moiety binds to at least one target antigen. In some embodiments, the non-CDR loop is modified to generate an antigen binding site specific for a target antigen. It is contemplated that various techniques can be used for modifying the non-CDR loop, e.g., site-directed mutagenesis, random mutagenesis, insertion of at least one amino acid that is foreign to the non-CDR loop amino acid sequence, amino acid substitution. An antigen peptide is inserted into a non-CDR loop, in some examples. In some examples, an antigenic peptide is substituted for the non-CDR loop. The modification, to generate an antigen binding site, is in some cases in only one non-CDR loop. In other instances, more than one non-CDR loop are modified. For instance, the modification is in any one of the non-CDR loops shown in FIG. 1, i.e., AB, CC′, C″ D, EF, and DE. In some cases, the modification is in the D-E loop. In some cases, the modification is in the CC′ loop. In other cases the modifications are in all four of AB, CC′, C″ D, EF loops. In certain examples, the binding moiety is bound to the target antigen binding domain via its AB, CC′, C″ D, or EF loop and is bound to a target antigen via its BC, C′C″, or FG loop. In certain examples, the binding moiety is bound to the target antigen binding domain via its AB, CC′, C″ D, and E-F loop and is bound to a target antigen via its BC, C′C″, and FG loop. In certain examples, the binding moiety is bound to the target antigen binding domain via one or more of AB, CC′, C″ D, and EF loop and is bound to a target antigen via one or more of B-C, C′C″, and FG loop. In certain examples, the binding moiety is bound to a target antigen via its AB, CC′, C″ D, or EF loop and is bound to the target antigen binding domain via its BC, C′C″, or FG loop. In certain examples, the binding moiety is bound to a target antigen via its AB, CC′, C″ D, and EF loop and is bound to the target antigen binding domain via its B-C, C′C″, and FG loop. In certain examples, the binding moiety is bound to a target antigen via one or more of A-B, CC′, C″D, and E-F loop and is bound to the target antigen binding protein, via one or more of BC, C′C″, and FG loop. In some embodiments, the CDRs provide a binding site for the at least one target antigen. Target antigens, in some cases, are expressed on the surface of a diseased cell or tissue, for example a tumor or a cancer cell. Target antigens include but are not limited to EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1.

In some embodiments, the binding moiety modifies a cellular microenvironment. In some embodiments, the microenvironment is a tumor microenvironment. In some cases, a tumor microenvironment contains inhibitory molecules which may bind to and inhibit immune cells. In some embodiments, the microenviromnemt modification domain inhibits the binding of inhibitory molecules, thereby increasing the activity of the immune cell.

In one embodiment, the binding moiety comprises any type of binding domain, including but not limited to, domains from a monoclonal antibody, a polyclonal antibody, a recombinant antibody, a human antibody, a humanized antibody. In some embodiments, the binding moiety is a single chain variable fragment (scFv), single-domain antibody such as a heavy chain variable domain (VH), a light chain variable domain (VL) and a variable domain (VHH) of camelid derived nanobody. In other embodiments, the binding moiety is a non-Ig binding domain, i.e., antibody mimetic, such as anticalins, affilins, affibody molecules, affimers, affitins, alphabodies, avimers, DARPins, fynomers, kunitz domain peptides, and monobodies.

It is contemplated herein that the binding moiety described herein comprises at least one cleavable linker. In one aspect, the cleavable linker comprises a polypeptide having a sequence recognized and cleaved in a sequence-specific manner. The binding moiety described herein, in some cases, comprises a protease cleavable linker recognized and cleaved in a sequence-specific manner. In some embodiments, the protease cleavable linker is recognized in a sequence-specific manner by a matrix metalloprotease (MMP), for example a MMP9. In some cases, the protease cleavable linker recognized by a MMP9 comprises a polypeptide having an amino acid sequence PR(S/T)(L/I)(S/T). In some cases, the protease cleavable linker recognized by a MMP9 comprises a polypeptide having an amino acid sequence LEATA. In some cases, the protease cleavable linker is recognized in a sequence-specific manner by a MMP11. In some cases, the protease cleavable linker recognized by a MMP11 comprises a polypeptide having an amino acid sequence GGAANLVRGG (SEQ ID NO: 5). In some cases, the protease cleavable linker is recognized by a protease disclosed in Table 1. In some cases, the protease cleavable linker recognized by a protease disclosed in Table 1 comprises a polypeptide having an amino acid sequence selected from a sequence disclosed in Table 1 (SEQ ID NOS: 1-42).

Proteases are proteins that cleave proteins, in some cases, in a sequence-specific manner. Proteases include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin K, Cathepsin L, kallikreins, hK1, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mir1-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, urokinase plasminogen activator (uPA), enterokinase, prostate-specific antigen (PSA, hK3), interleukin-1β converting enzyme, thrombin, FAP (FAP-α), dipeptidyl peptidase, and dipeptidyl peptidase IV (DPPIV/CD26).

TABLE 1
Exemplary Proteases and Protease
Recognition Sequences

| | Cleavage Domain | SEQ ID |
|---|---|---|
| Protease | Sequence | NO: |
| MMP7 | KRALGLPG | 1 |
| MMP7 | (DE)₈RPLALWRS(DR)₈ | 2 |
| MMP9 | PR(S/T)(L/I)(S/T) | 3 |
| MMP9 | LEATA | 4 |
| MMP11 | GGAANLVRGG | 5 |
| MMP14 | SGRIGFLRTA | 6 |
| MMP | PLGLAG | 7 |
| MMP | PLGLAX | 8 |
| MMP | PLGC(me)AG | 9 |
| MMP | ESPAYYTA | 10 |
| MMP | RLQLKL | 11 |
| MMP | RLQLKAC | 12 |
| MMP2, MMP9, MMP14 | EP(Cit)G(Hof)YL | 13 |
| Urokinase plasminogen | SGRSA | 14 |
| activator (uPA) | | |
| Urokinase plasminogen | DAFK | 15 |
| activator (uPA) | | |
| Urokinase plasminogen | GGGRR | 16 |
| activator (uPA) | | |
| Lysosomal Enzyme | GFLG | 17 |
| Lysosomal Enzyme | ALAL | 18 |
| Lysosomal Enzyme | FK | 19 |
| Cathepsin B | NLL | 20 |
| Cathepsin D | PIC(Et)FF | 21 |
| Cathepsin K | GGPRGLPG | 22 |
| Prostate Specific | HSSKLQ | 23 |
| Antigen | | |
| Prostate Specific | HSSKLQL | 24 |
| Antigen | | |
| Prostate Specific | HSSKLQEDA | 25 |
| Antigen | | |
| Herpes Simplex Virus | LVLASSSFGY | 26 |
| Protease | | |
| HIV Protease | GVSQNYPIVG | 27 |
| CMV Protease | GVVQASCRLA | 28 |
| Thrombin | F(Pip)RS | 29 |
| Thrombin | DPRSFL | 30 |
| Thrombin | PPRSFL | 31 |
| Caspase-3 | DEVD | 32 |
| Caspase-3 | DEVDP | 33 |
| Caspase-3 | KGSGDVEG | 34 |
| Interleukin 1β | GWEHDG | 35 |
| converting enzyme | | |
| Enterokinase | EDDDDKA | 36 |
| FAP | KQEQNPGST | 37 |
| Kallikrein 2 | GKAFRR | 38 |
| Plasmin | DAFK | 39 |
| Plasmin | DVLK | 40 |
| Plasmin | DAFK | 41 |
| TOP | ALLLALL | 42 |

Proteases are known to be secreted by some diseased cells and tissues, for example tumor or cancer cells, creating a microenvironment that is rich in proteases or a protease-rich microenvironment. In some case, the blood of a subject is rich in proteases. In some cases, cells surrounding the tumor secrete proteases into the tumor microenvironment. Cells surrounding the tumor secreting proteases include but are not limited to the tumor stromal cells, myofibroblasts, blood cells, mast cells, B cells, NK cells, regulatory T cells, macrophages, cytotoxic T lymphocytes, dendritic cells, mesenchymal stem cells, polymorphonuclear cells, and other cells. In some cases, proteases are present in the blood of a subject, for example proteases that target amino acid sequences found in microbial peptides. This feature allows for targeted therapeutics such as antigen binding proteins to have additional specificity because T cells will not be bound by the antigen binding protein except in the protease rich microenvironment of the targeted cells or tissue.

Other non-limiting examples of linkers that may be utilized in constructs described herein are provided in the Sequence Listing below.

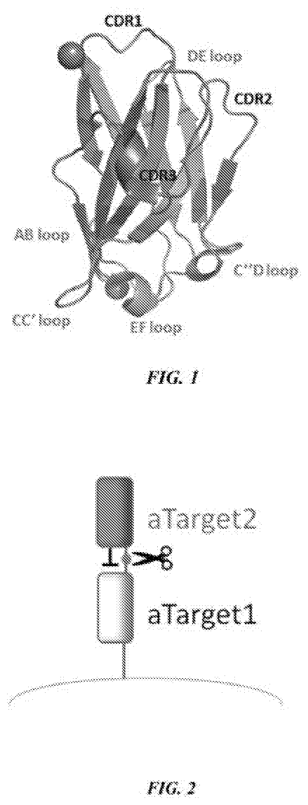

FIG. 2 shows a schematic representation of a portion of an example cleavable conditionally active receptor of the present disclosure. The conditionally active receptor comprises a target antigen binding domain (aTarget1), a cleavable linker, and a binding moiety (aTarget2). The target antigen binding domain has specificity for a first target, while the binding moiety has specifitity for a second target. The binding moiety also has a modified non-CDR loop, which inhibits binding of the target antigen binding domain to its target. Once cleaved at the cleavable linker, the binding moiety can be released, enabling binding of the target antigen binding domain.

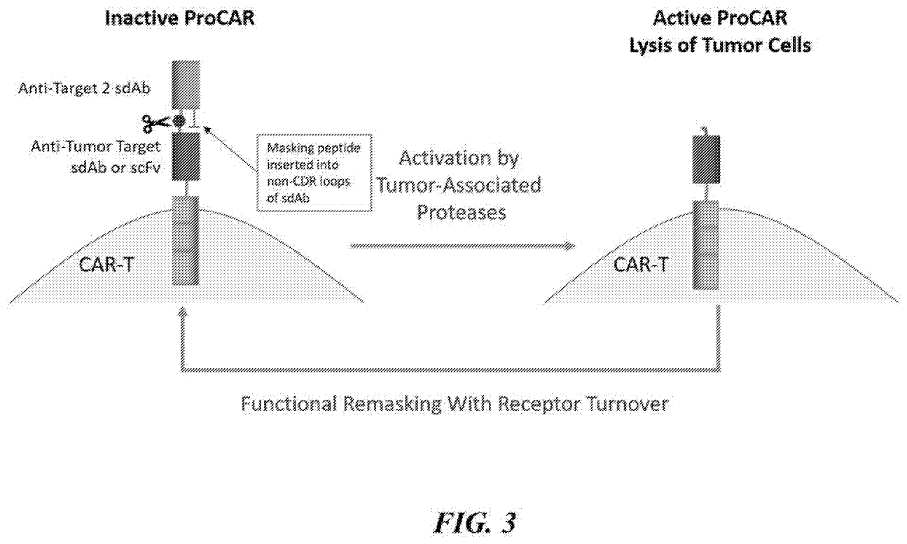

FIG. 3 shows a schematic representation of the activation of an example conditionally active receptor of the present disclosure. Inactive receptor (Inactive ProCAR) comprises a target antigen binding domain (Anti-Tumor Target sdAb or scFv) connected to a binding moiety (Anti-Target 2 sdAb) via a linker comprising a protease cleavage site. The binding moiety comprises a masking peptide, inserted into one or more non-CDR loops, such that the binding moiety binds to and inhibits the target antigen binding domain. In some embodiments, the binding moiety has specificity for a given target, as described further elsewhere herein. The receptor also comprises a transmembrane domain and an intracellular signaling domain. The receptor is provided in a T-cell (CAR-T). Upon exposure to a tumor environment, the protease cleavage site is cleaved by tumor-associated proteases, thereby activating the receptor, generating the active receptor which does not comprise the binding moeity. The receptor now comprises an active antigen-binding domain. As the receptor is internalized by the cell, new receptors are generated which comprise the binding moiety and are inactive.

### Targets of Conditionally Active Receptors

The conditionally active chimeric antigen receptors, T-cell receptor fusion proteins, and T-cell receptors described herein are activated by cleavage of the at least one cleavable linker of the binding moiety. It is contemplated that the activated receptor binds to a target antigen involved in and/or associated with a disease, disorder or condition. In particular, target antigens associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease are contemplated to be the target for the activated chimeric antigen receptors, T-cell receptor fusion proteins, and T-cell receptors disclosed herein.

In some embodiments, the target antigen is a tumor antigen expressed on a tumor cell. Tumor antigens are well known in the art and include, for example, EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. Exemplary sequences of tumor antigens include but are not limited to EpCAM (exemplary protein sequence comprises UniProtkB ID No. P16422), EGFR (exemplary protein sequence comprises UniProtkB ID No. P00533), HER-2 (exemplary protein sequence comprises UniProtkB ID No. P04626), HER-3 (exemplary protein sequence comprises UniProtkB ID No. P21860), c-Met (exemplary protein sequence comprises UniProtkB ID No. P08581), FoIR (exemplary protein sequence comprises UniProtkB ID No. P15238), PSMA (exemplary protein sequence comprises UniProtkB ID No. Q04609), CD38 (exemplary protein sequence comprises UniProtkB ID No. P28907), BCMA (exemplary protein sequence comprises UniProtkB ID No. Q02223), and CEA (exemplary protein sequence comprises UniProtkB ID No. P06731, 5T4 (exemplary protein sequence comprises UniProtkB ID No. Q13641), AFP (exemplary protein sequence comprises UniProtkB ID No. P02771), B7-H3 (exemplary protein sequence comprises UniProtkB ID No. Q5ZPR3), CDH-6 (exemplary protein sequence comprises UniProtkB ID No. P97326), CAIX (exemplary protein sequence comprises UniProtkB ID No. Q16790), CD117 (exemplary protein sequence comprises UniProtkB ID No. P10721), CD123 (exemplary protein sequence comprises UniProtkB ID No. P26951), CD138 (exemplary protein sequence comprises UniProtkB ID No. P18827), CD166 (exemplary protein sequence comprises UniProtkB ID No. Q13740), CD19 (exemplary protein sequence comprises UniProtkB ID No. P15931), CD20 (exemplary protein sequence comprises UniProtkB ID No. P11836), CD205 (exemplary protein sequence comprises UniProtkB ID No. 060449), CD22 (exemplary protein sequence comprises UniProtkB ID No. P20273), CD30 (exemplary protein sequence comprises UniProtkB ID No. P28908), CD33 (exemplary protein sequence comprises UniProtkB ID No. P20138), CD352 (exemplary protein sequence comprises UniProtkB ID No. Q96DU3), CD37 (exemplary protein sequence comprises UniProtkB ID No. P11049), CD44 (exemplary protein sequence comprises UniProtkB ID No. P16070), CD52 (exemplary protein sequence comprises UniProtkB ID No. P31358), CD56 (exemplary protein sequence comprises UniProtkB ID No. P13591), CD70 (exemplary protein sequence comprises UniProtkB ID No. P32970), CD71 (exemplary protein sequence comprises UniProtkB ID No. P02786), CD74 (exemplary protein sequence comprises UniProtkB ID No. P04233), CD79b (exemplary protein sequence comprises UniProtkB ID No. P40259), DLL3 (exemplary protein sequence comprises UniProtkB ID No. Q9NYJ7), EphA2 (exemplary protein sequence comprises UniProtkB ID No. P29317), FAP (exemplary protein sequence comprises UniProtkB ID No. Q12884), FGFR2 (exemplary protein sequence comprises UniProtkB ID No. P21802), FGFR3 (exemplary protein sequence comprises UniProtkB ID No. P22607), GPC3 (exemplary protein sequence comprises UniProtkB ID No. P51654), gpA33 (exemplary protein sequence comprises UniProtkB ID No. Q99795), FLT-3 (exemplary protein sequence comprises UniProtkB ID No. P36888), gpNMB (exemplary protein sequence comprises UniProtkB ID No. Q14956), HPV-16 E6 (exemplary protein sequence comprises UniProtkB ID No. P03126), HPV-16 E7 (exemplary protein sequence comprises UniProtkB ID No. P03129), ITGA2 (exemplary protein sequence comprises UniProtkB ID No. P17301), ITGA3 (exemplary protein sequence comprises UniProtkB ID No. P26006), SLC39A6 (exemplary protein sequence comprises UniProtkB ID No. Q13433), MAGE (exemplary protein sequence comprises UniProtkB ID No. Q9HC15), mesothelin (exemplary protein sequence comprises UniProtkB ID No. Q13421), Muc1 (exemplary protein sequence comprises UniProtkB ID No. P15941), Muc16 (exemplary protein sequence comprises UniProtkB ID No. Q8WX17), NaPi2b (exemplary protein sequence comprises UniProtkB ID No. 095436), Nectin-4 (exemplary protein sequence comprises UniProtkB ID No. Q96918), CDH-3 (exemplary protein sequence comprises UniProtkB ID No. Q8WX17), CDH-17 (exemplary protein sequence comprises UniProtkB ID No. E5RJT3), EPHB2 (exemplary protein sequence comprises UniProtkB ID No. P29323), ITGAV (exemplary protein sequence comprises UniProtkB ID No. P06756), ITGB6 (exemplary protein sequence comprises UniProtkB ID No. P18564), NY-ESO-1 (exemplary protein sequence comprises UniProtkB ID No. P78358), PRLR (exemplary protein sequence comprises UniProtkB ID No. P16471), PSCA (exemplary protein sequence comprises UniProtkB ID No. 043653), PTK7 (exemplary protein sequence comprises UniProtkB ID No. Q13308), ROR1 (exemplary protein sequence comprises UniProtkB ID No. Q01973), SLC44A4 (exemplary protein sequence comprises UniProtkB ID No. Q53GD3), SLITRK5 (exemplary protein sequence comprises UniProtkB ID No. Q81W52), SLITRK6 (exemplary protein sequence comprises UniProtkB ID No. Q9HY7), STEAP1 (exemplary protein sequence comprises UniProtkB ID No. Q9UHE8), TIM1 (exemplary protein sequence comprises UniProtkB ID No. Q96D42), Trop2 (exemplary protein sequence comprises UniProtkB ID No. P09758), or WT1 (exemplary protein sequence comprises UniProtkB ID No. P19544), or any combinations thereof.

In some embodiments, the target antigen is an immune checkpoint protein. Examples of immune checkpoint proteins include but are not limited to CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA. Exemplary sequences of immune checkpoint proteins include but are not limited to CD27 (exemplary protein sequence comprises UniProtkB ID No. P26842), CD137 (exemplary protein sequence comprises UniProtkB ID No. Q07011), 2B4 (exemplary protein sequence comprises UniProtkB ID No. Q9bZW8), TIGIT (exemplary protein sequence comprises UniProtkB ID No. Q495A1), CD155 (exemplary protein sequence comprises UniProtkB ID No. P15151), ICOS (exemplary protein sequence comprises UniProtkB ID No. Q9Y6W8), HVEM (exemplary protein sequence comprises UniProtkB ID No. 043557), CD40L (exemplary protein sequence comprises UniProtkB ID No. P29965), LIGHT (exemplary protein sequence comprises UniProtkB ID No. 043557), OX40 (exemplary protein sequence comprises UniProtkB ID No.), DNAM-1 (exemplary protein sequence comprises UniProtkB ID No. Q15762), PD-L1 (exemplary protein sequence comprises UniProtkB ID No. Q9ZQ7), PD1 (exemplary protein sequence comprises UniProtkB ID No. Q15116), PD-L2 (exemplary protein sequence comprises UniProtkB ID No. Q9BQ51), CTLA-4 (exemplary protein sequence comprises UniProtkB ID No. P16410), CD8 (exemplary protein sequence comprises UniProtkB ID No. P10966, P01732), CD40 (exemplary protein sequence comprises UniProtkB ID No. P25942), CEACAM1 (exemplary protein sequence comprises UniProtkB ID No. P13688), CD48 (exemplary protein sequence comprises UniProtkB ID No. P09326), CD70 (exemplary protein sequence comprises UniProtkB ID No. P32970), AA2AR (exemplary protein sequence comprises UniProtkB ID No. P29274), CD39 (exemplary protein sequence comprises UniProtkB ID No. P49961), CD73 (exemplary protein sequence comprises UniProtkB ID No. P21589), B7-H3 (exemplary protein sequence comprises UniProtkB ID No. Q5ZPR3), B7-H4 (exemplary protein sequence comprises UniProtkB ID No. Q7Z7D3), BTLA (exemplary protein sequence comprises UniProtkB ID No. Q76A9), IDO1 (exemplary protein sequence comprises UniProtkB ID No. P14902), IDO2 (exemplary protein sequence comprises UniProtkB ID No. Q6ZQW0), TDO (exemplary protein sequence comprises UniProtkB ID No. P48755), KIR (exemplary protein sequence comprises UniProtkB ID No. Q99706), LAG-3 (exemplary protein sequence comprises UniProtkB ID No. P18627), TIM-3 (also known as HAVCR2, exemplary protein sequence comprises UniProtkB ID No. Q8TDQ0), or VISTA (exemplary protein sequence comprises UniProtkB ID No. Q9D659).

In some embodiments, a target antigen is a cell surface molecule such as a protein, lipid or polysaccharide. In some embodiments, a target antigen is a on a tumor cell, virally infected cell, bacterially infected cell, damaged red blood cell, arterial plaque cell, or fibrotic tissue cell.

### Transmembrane Domain

The conditionally active chimeric antigen receptors, T-cell receptor fusion proteins, and T-cell receptors of the present disclosure include a transmembrane domain for insertion into a eukaryotic cell membrane. In some embodiments, the transmembrane domain is interposed between the target antigen binding domain and the intracellular domain. In some embodiments, the transmembrane domain is interposed between the target antigen binding domain and the costimulatory domain.

Any transmembrane (TM) domain that provides for insertion of a polypeptide into the cell membrane of a eukaryotic (e.g., mammalian) cell is suitable for use. As one non-limiting example, the TM sequence IYIWAPLAGTCGVLLLSLVITLYC (SEQ ID NO: 94) can be used. Additional non-limiting examples of suitable TM sequences include: a) CD8 beta derived: GLLVAGVLVLLVSLGVAIHLCC (SEQ ID NO: 95); b) CD4 derived: ALIVLGGVAGLLLFIGLGIFFCVRC (SEQ ID NO: 96); c) CD3 zeta derived: LCYLLDGILFIYGVILTALFLRV (SEQ ID NO: 97); d) CD28 derived: WVLVVVGGVLACYSLLVTVAFIIFWV (SEQ ID NO: 98); e) CD134 (OX40) derived: AAILGLGLVLGLLGPLAILLALYLL (SEQ ID NO: 99); and f) CD7 derived: ALPAALAVISFLLGLGLGVACVLA (SEQ ID NO: 100).

### Hinge Region

In some cases, the conditionally active chimeric antigen receptors, T-cell receptor fusion proteins, and T-cell receptors of the present disclosure comprise a hinge region (also referred to herein as a “spacer”), where the hinge region is interposed between the target antigen binding domain and the transmembrane domain. In some cases, the hinge region is an immunoglobulin heavy chain hinge region. In some cases, the hinge region is a hinge region polypeptide derived from a receptor (e.g., a CD8-derived hinge region).

The hinge region can have a length of from about 4 amino acids to about 50 amino acids (aa), e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

Suitable spacers can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Exemplary spacers include glycine polymers (G)n, glycine-serine polymers (including, for example, (GS)n, (GSGGS)n and (GGGS)n, where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary spacers comprise amino acid sequences including, but not limited to, GGSG, GGSGG, GSGSG, GSGGG, GGGSG, GSSSG, and the like.

Immunoglobulin hinge region amino acid sequences are known in the art; see, e.g., Tan et al. (1990) Proc. Natl. Acad. Sci. USA 87: 162; and Huck et al. (1986) Nucl. Acids Res. 14: 1779. As non-limiting examples, an immunoglobulin hinge region can include one of the following amino acid sequences: DKTHT; CPPC; CPEPKSCDTPPPCPR (SEQ ID NO: 101); see, e.g., Glaser et al. (2005) J. Biol. Chem. 280:41494); ELKTPLGDTTHT (SEQ ID NO: 102); KSCDKTHTCP (SEQ ID NO: 111); KCCVDCP (SEQ ID NO: 103); KYGPPCP (SEQ ID NO: 104); EPKSCDKTHTCPPCP (SEQ ID NO: 105); human IgG1 hinge); ERKCCVECPPCP (SEQ ID NO: 106); human IgG2 hinge); ELKTPLGDTTHTCPRCP (SEQ ID NO: 107); human IgG3 hinge); SPNMVPHAHHAQ (SEQ ID NO: 108); human IgG4 hinge); and the like.

In some embodiments, the hinge region comprises an amino acid sequence of a human IgG1, IgG2, IgG3, or IgG4, hinge region. The hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO: 109); see, e.g., Yan et al. (2012) J. Biol. Chem. 287:5891.

In some embodiments, the hinge region comprises an amino acid sequence derived from human CD8; e.g., the hinge region comprises the amino acid sequence: TTTPAPRPPTPAPTIASQPLSLRPEACRPAAGGAVHTRGLDFACD (SEQ ID NO: 110), or a variant thereof.

### Conditionally Active Chimeric Antigen Receptors

In one embodiment, the disclosure provides a conditionally active chimeric antigen receptor (CAR). A CAR generally comprises multiple domains, including a target antigen binding domain, a transmembrane domain, and an intracellular signaling domain. The conditionally active CAR of the present disclosure comprises multiple domains, including a binding moiety, a target antigen binding domain, a transmembrane domain, and an intracellular signaling domain. In some embodiments, the intracellular signaling domain is a signaling domain of a protein including, but not limited to, ZAP70, CD3 zeta, and 4-1BB.

In some embodiments, the conditionally active chimeric antigen receptor further comprises a costimulatory domain. In some embodiments, the costimulatory domain is a functional signaling domain of a protein including, but not limited to, OX40, CD2, CD27, CD28, CDS, ICAM-1, LFA-1 (CD11a/CD18), ICOS (CD278), and 4-1BB (CD137), and amino acid sequences thereof having at least one, two, or three modifications but not more than 20, 10, or 5 modifications thereto.

In some embodiments, the transmembrane domain comprises a transmembrane domain of a protein including, but not limited to, a TCR alpha chain, a TCR beta chain, a TCR zeta chain, a CD3 epsilon TCR subunit, a CD3 gamma TCR subunit, a CD3 delta TCR subunit, CD45, CD4, CDS, CD8, CD9, CD16, CD22, CD33, CD28, CD37, CD64, CD80, CD86, CD134, CD137, CD154, functional fragments thereof, and amino acid sequences thereof having at least one but not more than 20 modifications thereto.

In one aspect, the disclosure provides a cell (e.g., T-cell) engineered to express a CAR. In one aspect a cell is transformed with the CAR and the CAR is expressed on the cell surface. In some embodiments, the cell (e.g., T-cell) is transduced with a viral vector encoding a CAR. In some embodiments, the viral vector is a retroviral vector. In some embodiments, the viral vector is a lentiviral vector. In some such embodiments, the cell may stably express the CAR. In another embodiment, the cell (e.g., T cell) is transfected with a nucleic acid, e.g., mRNA, cDNA, DNA, encoding a CAR. In some such embodiments, the cell may transiently express the CAR.

### Conditionally Active T-Cell Receptor Fusion Proteins

In one embodiment, the disclosure provides a conditionally active T-cell receptor fusion protein. As used herein, a “T-cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T-cell.

The conditionally active TFP comprises a binding moiety, a target antigen binding domain, and a T-cell receptor subunit. In some embodiments, the T-cell receptor subunit further comprises at least a portion of a T-cell receptor extracellular domain, a transmembrane domain, and a T-cell receptor intracellular domain. In some embodiments, the transmembrane domain comprises a transmembrane domain of a protein including, but not limited to, a TCR alpha chain, a TCR beta chain, a TCR zeta chain, a CD3 epsilon TCR subunit, a CD3 gamma TCR subunit, a CD3 delta TCR subunit, CD45, CD4, CDS, CD8, CD9, CD16, CD22, CD33, CD28, CD37, CD64, CD80, CD86, CD134, CD137, CD154, functional fragments thereof, or amino acid sequences thereof having at least one, two, or three modifications but not more than 20, 10, or 5 modifications thereto.

In some embodiments, the T-cell receptor intracellular domain comprises a stimulatory domain. The stimulatory domain may be from T-cell receptor subunit, including but not limited to the beta subunit, alpha subunit, delta subunit, gamma subunit, epsilon subunit, or a combination thereof. In some embodiments, the stimulatory domain comprises an immunoreceptor tyrosine-based activation motif (ITAM) or portion thereof including, but not limited to, CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b 1 chain, Fc gamma receptor 2b2 chain, Fc gamma receptor 3a chain, Fc gamma receptor 3b chain, Fc beta receptor 1 chain, TYROBP (DAP12), CDS, CD16a, CD16b, CD22, CD23, CD32, CD64, CD79a, CD79b, CD89, CD278, CD66d, functional fragments thereof, and amino acid sequences thereof having at least one, two, or three modifications but not more than 20, 10, or 5 modifications thereto.

In some embodiments, the conditionally active TFP further comprises a costimulatory domain. In some embodiments, the costimulatory domain is a functional signaling domain of a protein including, but not limited to, OX40, CD2, CD27, CD28, CDS, ICAM-1, LFA-1 (CD11a/CD18), ICOS (CD278), and 4-1BB (CD137), and amino acid sequences thereof having at least one, two, or three modifications but not more than 20, 10, or 5 modifications thereto.

In some embodiments, the target antigen binding domain is connected to the T-cell receptor extracellular domain by a linker sequence. In some instances, the encoded linker sequence comprises (G₄S)n, wherein n=1 to 4. In some instances, the encoded linker sequence comprises a long linker (LL) sequence. In some instances, the encoded long linker sequence comprises (G4S)n, wherein n=2 to 4. In some instances, the encoded linker sequence comprises a short linker (SL) sequence. In some instances, the encoded short linker sequence comprises (G₄S)n, wherein n=1 to 3.

In one aspect, the disclosure provides a cell (e.g., T-cell) engineered to express a conditionally active T-cell receptor fusion protein (TFP). In one aspect a cell is transformed with the conditionally active TFP and the conditionally active TFP is expressed on the cell surface. In some embodiments, the cell (e.g., T-cell) is transduced with a viral vector encoding a conditionally active TFP. In some embodiments, the viral vector is a retroviral vector. In some embodiments, the viral vector is a lentiviral vector. In some such embodiments, the cell may stably express the conditionally active TFP. In another embodiment, the cell (e.g., T cell) is transfected with a nucleic acid, e.g., mRNA, cDNA, DNA, encoding a conditionally active TFP. In some such embodiments, the cell may transiently express the conditionally active TFP.

### Conditionally Active T-Cell Receptors

In one embodiment, the disclosure provides a conditionally active T-cell receptor. A T-cell receptor generally comprises multiple subunits, including alpha, beta, delta, gamma, epsilon, and zeta subunits. The conditionally active T-cell receptor of the present disclosure comprises a binding moiety. In some embodiments, the binding moiety is attached to a T-cell receptor subunit including, but not limited to, the alpha subunit, beta subunit, or a combination thereof.

In some embodiments, the binding moiety is capable of masking the binding of the T-cell receptor to its target. In some embodiments, the binding moiety binds to T-cell receptor. In some embodiments, a non-CDR loop provides a binding site for binding of the moiety to the T-cell receptor. In some embodiments, the non-CDR loop provides a binding site specific for T-cell receptor alpha, T-cell receptor beta, or a combination thereof. In some embodiments, the binding moiety masks binding of the T-cell receptor to its target, e.g. via steric occlusion, via specific intermolecular interactions.

In one aspect, the disclosure provides a cell (e.g., T-cell) engineered to express a conditionally active T-cell receptor (TCR). In one aspect a cell is transformed with the conditionally active TCR and the conditionally active TCR is expressed on the cell surface. In some embodiments, the cell (e.g., T-cell) is transduced with a viral vector encoding a conditionally active TCR. In some embodiments, the viral vector is a retroviral vector. In some embodiments, the viral vector is a lentiviral vector. In some such embodiments, the cell may stably express the conditionally active TCR. In another embodiment, the cell (e.g., T cell) is transfected with a nucleic acid, e.g., mRNA, cDNA, DNA, encoding a conditionally active TCR. In some such embodiments, the cell may transiently express the conditionally active TCR.

### Cells

In one embodiment, the present disclosure provides a cell comprising the conditionally active chimeric antigen receptor, conditionally active T-cell receptor fusion protein, or conditionally active T-cell receptor of the present disclosure. The cell may be a mammalian cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, HuT-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell or progenitor cell obtained from an individual.

### Methods of Generating a Cell Comprising a Conditionally Active Receptor

The present disclosure provides a method of generating a cell comprising a conditionally active chimeric antigen receptor, T-cell receptor fusion protein, or T-cell receptor. The method generally involves genetically modifying a mammalian cell with an expression vector, or an RNA (e.g., in vitro transcribed RNA), comprising nucleotide sequences encoding a conditionally active chimeric antigen receptor, T-cell receptor fusion protein, or T-cell receptor of the present disclosure. The genetic modification can be carried out in vivo, in vitro, or ex vivo. The cell can be, for example, an immune cell (e.g., a T lymphocyte or NK cell), a stem cell, or a progenitor cell.

In some cases, the genetic modification is carried out ex vivo. For example, a T lymphocyte, a stem cell, or an NK cell is obtained from an individual; and the cell obtained from the individual is genetically modified to express a conditionally active chimeric antigen receptor, T-cell receptor fusion protein, or T-cell receptor of the present disclosure.

### Sources of T-Cells

In some embodiments, a source of T-cells is obtained from a subject. The term “subject” is intended to include living organisms in which an immune response can be elicited (e.g., mammals). Examples of subjects include humans, dogs, cats, mice, rats, and transgenic species thereof. T-cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T-cell lines available in the art, may be used. In certain embodiments of the present disclosure, T-cells can be obtained from a unit of blood collected from a subject using any number of techniques known to the skilled artisan, such as FICOLL™ separation. In one embodiment, cells from the circulating blood of an individual are obtained by apheresis. The apheresis product typically contains lymphocytes, including T-cells, monocytes, granulocytes, B cells, other nucleated white blood cells, red blood cells, and platelets. In one embodiment, the cells collected by apheresis are washed to remove the plasma fraction and to place the cells in an appropriate buffer or media for subsequent processing steps. In one embodiment of the disclosure, the cells are washed with phosphate buffered saline (PBS). In an alternative embodiment, the wash solution lacks calcium and may lack magnesium or may lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In one embodiment, T-cells are isolated from peripheral blood lymphocytes by lysing the red blood cells and depleting the monocytes, for example, by centrifugation through a PERCOLL™ gradient or by counterflow centrifugal elutriation. A specific subpopulation of T-cells, such as CD3+, CD28+, CD4+, CD8+, CD45RA+, and CD45RO+ T-cells, can be further isolated by positive or negative selection techniques. For example, in one embodiment, T-cells are isolated by incubation with anti-CD3/anti-CD28 (e.g., 3×28)-conjugated beads, such as DYNABEADS® M-450 CD3/CD28 T, for a time period sufficient for positive selection of the desired T-cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours. In one embodiment, the incubation time period is 24 hours. Longer incubation times may be used to isolate T-cells in any situation where there are few T-cells as compared to other cell types, such in isolating tumor infiltrating lymphocytes (TIL) from tumor tissue or from immunocompromised individuals. Further, use of longer incubation times can increase the efficiency of capture of CD8+ T-cells. Thus, by simply shortening or lengthening the time T-cells are allowed to bind to the CD3/CD28 beads and/or by increasing or decreasing the ratio of beads to T-cells (as described further herein), subpopulations of T-cells can be preferentially selected for or against at culture initiation or at other time points during the process. Additionally, by increasing or decreasing the ratio of anti-CD3 and/or anti-CD28 antibodies on the beads or other surface, subpopulations of T-cells can be preferentially selected for or against at culture initiation or at other desired time points. Multiple rounds of selection can also be used in the context of this disclosure. In certain embodiments, it may be desirable to perform the selection procedure and use the “unselected” cells in the activation and expansion process. “Unselected” cells can also be subjected to further rounds of selection.

Enrichment of a T-cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T-cells which typically express CD4+, CD25+, CD62Lhi, GITR+, and FoxP3+. Alternatively, in certain embodiments, T regulatory cells are depleted by anti-CD25 conjugated beads or other similar method of selection.

In one embodiment, a T-cell population can be selected that expresses one or more of IFN-γ, TNFα, IL-17A, IL-2, IL-3, IL-4, GM-CSF, granzyme B, and perforin, or other appropriate molecules, e.g., other cytokines. Methods for screening for cell expression can be determined, e.g., by the methods described in PCT Publication No. WO 2013/126712.

For isolation of a desired population of cells by positive or negative selection, the concentration of cells and surface (e.g., particles such as beads) can be varied. In certain embodiments, it may be desirable to significantly decrease the volume in which beads and cells are mixed together (e.g., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet one embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T-cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T-cells that normally have weaker CD28 expression.

In another embodiment, it may be desirable to use lower concentrations of cells. By significantly diluting the mixture of T-cells and surface (e.g., particles such as beads), interactions between the particles and cells is minimized. This selects for cells that express high amounts of desired antigens to be bound to the particles. For example, CD4+ T-cells express higher levels of CD28 and are more efficiently captured than CD8+ T-cells in dilute concentrations. In one embodiment, the concentration of cells used is 5×10e6/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T-cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present disclosure.

Also contemplated in the context of the disclosure is the collection of blood samples or apheresis product from a subject at a time period prior to when the expanded cells as described herein might be needed. As such, the source of the cells to be expanded can be collected at any time point necessary, and desired cells, such as T-cells, isolated and frozen for later use in T-cell therapy for any number of diseases or conditions that would benefit from T-cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T-cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further embodiment of the present disclosure, T-cells are obtained from a patient directly following treatment that leaves the subject with functional T-cells. In this regard, it has been observed that following certain cancer treatments, in particular treatments with drugs that damage the immune system, shortly after treatment during the period when patients would normally be recovering from the treatment, the quality of T-cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T-cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T-cells, B cells, dendritic cells, and other cells of the immune system.

### Activation and Expansion of T-Cells

T-cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T-cells of the disclosure may be expanded by contact with a surface having attached thereto an agent that stimulates a CD3/TCR complex associated signal and a ligand that stimulates a costimulatory molecule on the surface of the T-cells. In particular, T-cell populations may be stimulated as described herein, such as by contact with an anti-CD3 antibody, or antigen binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T-cells, a ligand that binds the accessory molecule is used. For example, a population of T-cells can be contacted with an anti-CD3 antibody and an anti-CD28 antibody, under conditions appropriate for stimulating proliferation of the T-cells. To stimulate proliferation of either CD4+ T-cells or CD8+ T-cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

In certain embodiments, the primary stimulatory signal and the costimulatory signal for the T-cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the costimulatory signal is bound to a cell surface and the agent providing the primary activation signal is in solution or coupled to a surface. In certain embodiments, both agents can be in solution. In one embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T-cells in the present disclosure.

In one embodiment, the two agents are immobilized on beads, either on the same bead, i.e., “cis,” or to separate beads, i.e., “trans.” By way of example, the agent providing the primary activation signal is an anti-CD3 antibody or an antigen binding fragment thereof and the agent providing the costimulatory signal is an anti-CD28 antibody or antigen binding fragment thereof; and both agents are co-immobilized to the same bead in equivalent molecular amounts. In one embodiment, a 1:1 ratio of each antibody bound to the beads for CD4+ T-cell expansion and T-cell growth is used. In certain embodiments of the present disclosure, a ratio of anti CD3:CD28 antibodies bound to the beads is used such that an increase in T-cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one embodiment of the present disclosure, more anti-CD28 antibody is bound to the particles than anti-CD3 antibody, i.e., the ratio of CD3:CD28 is less than one. In certain embodiments of the disclosure, the ratio of anti CD28 antibody to anti CD3 antibody bound to the beads is greater than 2:1. In one particular embodiment, a 1:100 CD3:CD28 ratio of antibody bound to beads is used. In one embodiment, a 1:75 CD3:CD28 ratio of antibody bound to beads is used. In a further embodiment, a 1:50 CD3:CD28 ratio of antibody bound to beads is used. In one embodiment, a 1:30 CD3:CD28 ratio of antibody bound to beads is used. In one embodiment, a 1:10 CD3:CD28 ratio of antibody bound to beads is used. In one embodiment, a 1:3 CD3:CD28 ratio of antibody bound to the beads is used. In yet one embodiment, a 3:1 CD3:CD28 ratio of antibody bound to the beads is used.

Ratios of particles to cells from 1:500 to 500:1 and any integer values in between may be used to stimulate T-cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T-cells. The ratio of anti-CD3- and anti-CD28-coupled particles to T-cells that result in T-cell stimulation can vary as noted above, however certain values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T-cell. In one embodiment, a ratio of particles to cells of 1:1 or less is used. In one particular embodiment, a particle:cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In one embodiment, particles are added on a daily or every other day basis to a final ratio of 1:1 on the first day, and 1:5 on the third and fifth days of stimulation. In one embodiment, the ratio of particles to cells is 2:1 on the first day of stimulation and adjusted to 1:10 on the third and fifth days of stimulation. In one embodiment, particles are added on a daily or every other day basis to a final ratio of 1:1 on the first day, and 1:10 on the third and fifth days of stimulation. One of skill in the art will appreciate that a variety of other ratios may be suitable for use in the present disclosure. In particular, ratios will vary depending on particle size and on cell size and type.

In further embodiments of the present disclosure, the cells, such as T-cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T-cells. In one embodiment the cells (for example, 104 to 109T-cells) and beads (for example, DYNABEADS® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present disclosure. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In one embodiment, greater than 100 million cells/ml is used. In a further embodiment, a concentration of cells of 10, 15, 20, 25, 30, 35, 40, 45, or 50 million cells/ml is used. In yet one embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T-cells. Such populations of cells may have therapeutic value and would be desirable to obtain in certain embodiments. For example, using high concentration of cells allows more efficient selection of CD8+ T-cells that normally have weaker CD28 expression.

In one embodiment of the present disclosure, the mixture may be cultured for several hours (about 3 hours) to about 14 days or any hourly integer value in between. In one embodiment, the mixture may be cultured for 21 days. In one embodiment of the disclosure the beads and the T-cells are cultured together for about eight days. In one embodiment, the beads and T-cells are cultured together for 2-3 days. Several cycles of stimulation may also be desired such that culture time of T-cells can be 60 days or more. Conditions appropriate for T-cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T-cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T-cells that have been exposed to varied stimulation times may exhibit different characteristics. For example, typical blood or apheresed peripheral blood mononuclear cell products have a helper T-cell population (TH, CD4+) that is greater than the cytotoxic or suppressor T-cell population (TC, CD8+). Ex vivo expansion of T-cells by stimulating CD3 and CD28 receptors produces a population of T-cells that prior to about days 8-9 consists predominately of TH cells, while after about days 8-9, the population of T-cells comprises an increasingly greater population of TC cells.

### Activated Conditionally Active Receptors

The conditionally active chimeric antigen receptors, T-cell receptor fusion proteins, and T-cell receptors described herein are activated by cleavage of cleavable linkers the binding moiety and the target antigen binding domain. It is contemplated that the activated conditionally active chimeric antigen receptors, T-cell receptor fusion proteins, and T-cell receptors bind to a target antigen involved in and/or associated with a disease, disorder or condition. In particular, target antigens associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease are contemplated to be the target for the activated chimeric antigen receptors, T-cell receptor fusion proteins, and T-cell receptors disclosed herein. In some embodiments, the target antigen is a tumor antigen expressed on a tumor cell. In some embodiments, a target antigen is a on a tumor cell, virally infected cell, bacterially infected cell, damaged red blood cell, arterial plaque cell, or inflamaed or fibrotic tissue cell.

In some embodiments, a target antigen is a cell surface molecule such as a protein, lipid or polysaccharide. In some embodiments, a target antigen is an immune checkpoint protein.

Target antigens, in some cases, are expressed on the surface of a diseased cell or tissue, for example a tumor or a cancer cell. Examples of target antigens include but are not limited to EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, a target antigen comprises an immune checkpoint protein. Examples of immune checkpoint proteins include but are not limited to CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDOL IDO2, TDO, KIR, LAG-3, TIM-3, or VISTA. Inhibitory immune checkpoint proteins to be inhibited in activating an immune response include but are not limited to A2AR, CD39, CD73, B7-H3, B7-H4, BTLA, CTLA-4, IDOL IDO2, TDO, KIR, LAG3, PD-1, PD-L1, TIM-3, and VISTA. In some embodiments, binding of the conditionally active chimeric antigen receptors, T-cell receptor fusion proteins, and T-cell receptors to an immune checkpoint target protein is dependent upon protease cleavage of the inhibitory domain which restricts binding of the protein to the immune checkpoint target protein only in the microenvironment of a diseased cell or tissue with elevated levels of proteases, for example in a tumor microenvironment.

In some embodiments, the target antigen binding domain of the conditionally active chimeric antigen receptors, T-cell receptor fusion proteins, and T-cell receptors described herein specifically binds to A2AR. In some embodiments, the target antigen binding domain of the conditionally active chimeric antigen receptors, T-cell receptor fusion proteins, and T-cell receptors described herein specifically binds to B7-H3. In some embodiments, the target antigen binding domain of the conditionally active chimeric antigen receptors, T-cell receptor fusion proteins, and T-cell receptors described herein specifically binds to B7-H4. In some embodiments, the target antigen binding domain of the conditionally active chimeric antigen receptors, T-cell receptor fusion proteins, and T-cell receptors described herein specifically binds to BTLA. In some embodiments, the target antigen binding domain of the conditionally active chimeric antigen receptors, T-cell receptor fusion proteins, and T-cell receptors described herein specifically binds to CTLA-4. In some embodiments, the target antigen binding domain of the conditionally active chimeric antigen receptors, T-cell receptor fusion proteins, and T-cell receptors described herein specifically binds to IDO. In some embodiments, the target antigen binding domain of the conditionally active chimeric antigen receptors, T-cell receptor fusion proteins, and T-cell receptors described herein specifically binds to KIR. In some embodiments, the target antigen binding domain of the conditionally active chimeric antigen receptors, T-cell receptor fusion proteins, and T-cell receptors described herein specifically binds to LAG3. In some embodiments, the target antigen binding domain of the conditionally active chimeric antigen receptors, T-cell receptor fusion proteins, and T-cell receptors described herein specifically binds to PD-1. In some embodiments, the target antigen binding domain of the conditionally active chimeric antigen receptors, T-cell receptor fusion proteins, and T-cell receptors described herein specifically binds to PD-L1. In some embodiments, the target antigen binding domain of the conditionally active chimeric antigen receptors, T-cell receptor fusion proteins, and T-cell receptors described herein specifically binds to TIM-3. In some embodiments, the target antigen binding domain of the conditionally active chimeric antigen receptors, T-cell receptor fusion proteins, and T-cell receptors described herein specifically binds to VISTA.

In some embodiments, a target antigen is a viral antigen. Examples of viral antigens include, but are not limited to, Hepatitis Viruses, Flaviviruses, Westnile Virus, Ebola Virus, Pox-Virus, Smallpox Virus, Measles Virus, Herpes Virus, Adenovirus, Papilloma Virus, Polyoma Virus, Parvovirus, Rhinovirus, Coxsackie virus, Polio Virus, Echovirus, Japanese Encephalitis virus, Dengue Virus, Tick Burne Encephalitis Virus, Yellow Fever Virus, Coronavirus, respiratory syncytial virus, parainfluenza virus, La Crosse Virus, Lassa Virus, Rabies Viruses, and Rotavirus antigens.

### Binding Protein Variants

As used herein, the term “binding protein variants” refers to variants and derivatives of conditionally active chimeric antigen receptors, T-cell receptor fusion proteins, and T-cell receptors described herein. In certain embodiments, amino acid sequence variants of the conditionally active chimeric antigen receptors, T-cell receptor fusion proteins, and T-cell receptors described herein are contemplated. For example, in certain embodiments amino acid sequence variants of the conditionally active chimeric antigen receptors, T-cell receptor fusion proteins, and T-cell receptors described herein are contemplated to improve the binding affinity and/or other biological properties of the receptor. Exemplary method for preparing amino acid variants include, but are not limited to, introducing appropriate modifications into the nucleotide sequence encoding the receptor, or by peptide synthesis. Such modifications include, for example, deletions from, and/or insertions into and/or substitutions of residues within the amino acid sequences of the receptor.

Any combination of deletion, insertion, and substitution can be made to the various domains to arrive at the final construct, provided that the final construct possesses the desired characteristics, e.g., antigen-binding. In certain embodiments, binding protein variants having one or more amino acid substitutions are provided. Sites of interest for substitution mutagenesis include the CDRs and framework regions. Amino acid substitutions may be introduced into the variable domains of the target-binding protein of interest and the products screened for a desired activity, e.g., retained/improved antigen binding, decreased immunogenicity, or complement dependent cytotoxicity (CDC). Both conservative and non-conservative amino acid substitutions are contemplated for preparing the receptor variants.

In another example of a substitution to create a variant conditionally active chimeric antigen receptors, T-cell receptor fusion proteins, and T-cell receptors, one or more hypervariable region residues of a parent receptor are substituted. In general, variants are then selected based on improvements in desired properties compared to a parent receptor, for example, increased affinity, reduced affinity, reduced immunogenicity, increased pH dependence of binding. For example, an affinity matured variant receptor can be generated, e.g., using phage display-based affinity maturation techniques such as those described herein and known in the field.

### Affinity Maturation

In designing binding proteins for therapeutic applications, it is desirable to create proteins that, for example, modulate a functional activity of a target, and/or improved binding proteins such as binding proteins with higher specificity and/or affinity and/or and binding proteins that are more bioavailable, or stable or soluble in particular cellular or tissue environments.

The binding moiety described in the present disclosure exhibit improved the binding affinities towards the target binding domain, for example a tumor antigen expressed on a cell surface. In some embodiments, the binding moiety of the present disclosure is affinity matured to increase its binding affinity to the target binding domain, using any known technique for affinity-maturation (e.g., mutagenesis, chain shuffling, CDR amino acid substitution). Amino acid substitutions may be conservative or semi-conservative. For example, the amino acids glycine, alanine, valine, leucine and isoleucine can often be substituted for one another (amino acids having aliphatic side chains). Of these possible substitutions, typically glycine and alanine are used to substitute for one another since they have relatively short side chains and valine, leucine and isoleucine are used to substitute for one another since they have larger aliphatic side chains which are hydrophobic. Other amino acids which may often be substituted for one another include but are not limited to: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur-containing side chains). In some embodiments, the binding moiety is isolated by screening combinatorial libraries, for example, by generating phage display libraries and screening such libraries for antibodies possessing the desired binding characteristics towards a target, for example a target antigen binding domain, a tumor antigen, or any other target antigen.

### Conditionally Active Receptor Protein Modifications

The receptors described herein encompass derivatives or analogs in which (i) an amino acid is substituted with an amino acid residue that is not one encoded by the genetic code, (ii) the mature polypeptide is fused with another compound such as polyethylene glycol, or (iii) additional amino acids are fused to the protein, such as a leader or secretory sequence or a sequence to block an immunogenic domain and/or for purification of the protein.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in the conditionally active chimeric antigen receptors, T-cell receptor fusion proteins, and T-cell receptors described herein, including the peptide backbone, the amino acid side-chains, and the amino or carboxyl termini. Certain common peptide modifications that are useful for modification of the conditionally active binding proteins include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

### Polynucleotides Encoding Receptors

Also provided, in some embodiments, are polynucleotide molecules encoding the conditionally active chimeric antigen receptor, T-cell receptor fusion protein, or modified T-cell receptor as described herein. In some embodiments, the polynucleotide molecules are provided as a DNA construct. In other embodiments, the polynucleotide molecules are provided as a messenger RNA transcript.

The polynucleotide molecules are constructed by known methods such as by combining the genes encoding the various binding domains (e.g. binding moiety, target antigen binding domain, transmembrane domain, intracellular signaling domain, etc.) either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. Depending on the vector system and host utilized, any number of suitable transcription and translation elements, including constitutive and conditionally active promoters, may be used. The promoter is selected such that it drives the expression of the polynucleotide in the respective host cell.

In some embodiments, the polynucleotide is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described conditionally active chimeric antigen receptor, T-cell receptor fusion protein, or T-cell receptor. Examples of expression vectors for expression in E. coli are pSKK (Le Gall et al., J Immunol Methods. (2004) 285(1):111-27) or pcDNA5 (Invitrogen) for expression in mammalian cells.

Thus, the conditionally active chimeric antigen receptor, T-cell receptor fusion protein, and T-cell receptor as described herein, in some embodiments, are produced by introducing a vector encoding the protein as described above into a host cell and culturing said host cell under conditions whereby the protein domains are expressed.

### Production of Conditionally Active Receptor

Disclosed herein, in some embodiments, is a process for the production of conditionally active chimeric antigen receptors, T-cell receptor fusion proteins, and T-cell receptors of the present disclosure. In some embodiments, the process comprises culturing a host transformed or transfected with a vector comprising a nucleic acid sequence encoding a conditionally active chimeric antigen receptor, T-cell receptor fusion protein, or T-cell receptor under conditions allowing the expression of the conditionally active chimeric antigen receptor, T-cell receptor fusion protein, or T-cell receptor and recovering and purifying the produced protein from the culture.

In an additional embodiment is provided a process directed to improving one or more properties, e.g., affinity, stability, heat tolerance, cross-reactivity, etc., of the conditionally active chimeric antigen receptors, T-cell receptor fusion proteins, and T-cell receptors described herein, compared to a reference binding compound. In some embodiments, a plurality of single-substitution libraries is provided each corresponding to a different domain, or amino acid segment of the conditionally active chimeric antigen receptor, T-cell receptor fusion protein, or T-cell receptor or reference binding compound such that each member of the single-substitution library encodes only a single amino acid change in its corresponding domain, or amino acid segment. Typically, this allows all of the potential substitutions in a large protein or protein binding site to be probed with a few small libraries. In some embodiments, the plurality of domains forms or covers a contiguous sequence of amino acids of the conditionally active chimeric antigen receptor, T-cell receptor fusion protein, or T-cell receptor or a reference binding compound. Nucleotide sequences of different single-substitution libraries overlap with the nucleotide sequences of at least one other single-substitution library. In some embodiments, a plurality of single-substitution libraries are designed so that every member overlaps every member of each single-substitution library encoding an adjacent domain.

Binding proteins expressed from such single-substitution libraries are separately selected to obtain a subset of variants in each library which has properties at least as good as those of the reference binding compound and whose resultant library is reduced in size. Generally, the number of nucleic acids encoding the selected set of binding compounds is smaller than the number of nucleic acids encoding members of the original single-substitution library. Such properties include, but are not limited to, affinity to a target compound, stability with respect to various conditions such as heat, high or low pH, enzymatic degradation, cross-reactivity to other proteins and the like. The selected compounds from each single-substitution library are referred to herein interchangeably as “pre-candidate compounds,” or “pre-candidate proteins.” Nucleic acid sequences encoding the pre-candidate compounds from the separate single-substitution libraries are then shuffled in a PCR to generate a shuffled library, using PCR-based gene shuffling techniques.

An exemplary work flow of the screening process is described herein. Libraries of pre-candidate compounds are generated from single substitution libraries and selected for binding to the target protein(s), after which the pre-candidate libraries are shuffled to produce a library of nucleic acids encoding candidate compounds which, in turn, are cloned into a convenient expression vector, such as a phagemid expression system. Phage expressing candidate compounds then undergo one or more rounds of selection for improvements in desired properties, such as binding affinity to a target molecule. Target molecules may be adsorbed or otherwise attached to a surface of a well or other reaction container, or target molecules may be derivatized with a binding moiety, such as biotin, which after incubation with candidate binding compounds may be captured with a complementary moiety, such as streptavidin, bound to beads, such as magnetic beads, for washing. In exemplary selection regimens, the candidate binding compounds undergo a wash step so that only candidate compounds with very low dissociation rates from a target molecule are selected. Exemplary wash times for such embodiments are about 10 minutes, about 15 minutes, about 20 minutes, about 20 minutes, about 30 minutes, about 35 minutes, about 40 minutes, about 45 minutes, about 50 minutes, about 55 mins, about 1 hour, about 2 hours, about 3 hours, about 4 hours, about 5 hours, about 6 hours, about 7 hours, about 8 hours; or in other embodiments, about 24 hours; or in other embodiments, about 48 hours; or in other embodiments, about 72 hours. Isolated clones after selection are amplified and subjected to an additional cycle of selection or analyzed, for example by sequencing and by making comparative measurements of binding affinity towards their target, for example, by ELISA, surface plasmon resonance (SPR), bio-layer interferometry (e.g., OCTET® system, Pall Life Sciences, ForteBio, Menlo Park, Calif.) or the like.

### Pharmaceutical Compositions

Also provided, in some embodiments, are pharmaceutical compositions comprising a therapeutically effective amount of cells (e.g. T-cells) comprising a conditionally active chimeric antigen receptor, T-cell receptor fusion protein, or modified T-cell receptor of the present disclosure, and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The cells described herein are contemplated for use as a medicament. Administration is effected by different ways, e.g., by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

### Methods of Treatment

Also provided herein, in some embodiments, are methods and uses for stimulating the immune system of an individual in need thereof comprising administration of cells (e.g. T-cells) comprising a conditionally active chimeric antigen receptor, T-cell receptor fusion protein, or modified T-cell receptor as described herein. In some instances, administration induces and/or sustains cytotoxicity towards a cell expressing a target antigen. In some instances, the cell expressing a target antigen is a cancer or tumor cell, a virally infected cell, a bacterially infected cell, an autoreactive T or B cell, damaged red blood cells, arterial plaques, or fibrotic tissue. In some embodiments, the target antigen is an immune checkpoint protein.

Also provided herein are methods and uses for a treatment of a disease, disorder or condition associated with a target antigen comprising administering to an individual in need thereof cells (e.g. T-cells) comprising a conditionally active chimeric antigen receptor, T-cell receptor fusion protein, or modified T-cell receptor as described herein. Diseases, disorders or conditions associated with a target antigen include, but are not limited to, viral infection, bacterial infection, auto-immune disease, transplant rejection, atherosclerosis, or fibrosis. In other embodiments, the disease, disorder or condition associated with a target antigen is a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In one embodiment, the disease, disorder or condition associated with a target antigen is cancer. In one instance, the cancer is a hematological cancer. In another instance, the cancer is a melanoma. In a further instance, the cancer is non-small cell lung cancer. In yet further instance, the cancer is breast cancer.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

In some embodiments of the methods described herein, the cells described herein are administered in combination with an agent for treatment of the particular disease, disorder or condition. Agents include but are not limited to, therapies involving antibodies, small molecules (e.g., chemotherapeutics), hormones (steroidal, peptide, and the like), radiotherapies (y-rays, X-rays, and/or the directed delivery of radioisotopes, microwaves, UV radiation and the like), gene therapies (e.g., antisense, retroviral therapy and the like) and other immunotherapies. In some embodiments, the cells comprising the conditionally active chimeric antigen receptor, T-cell receptor fusion protein, or modified T-cell receptor described herein are administered in combination with anti-diarrheal agents, anti-emetic agents, analgesics, opioids and/or non-steroidal anti-inflammatory agents. In some embodiments, the cells comprising the conditionally active chimeric antigen receptor, T-cell receptor fusion protein, or modified T-cell receptor described herein are administered before, during, or after surgery.

### EXAMPLES

The examples below further illustrate the described embodiments without limiting the scope of the disclosure.

### Example 1: Construction of an Exemplary Binding Moiety which Binds to IL-2 and a Target Antigen Binding Domain Whose Target is EGFR

The sequence of an engineered protein scaffold comprising CDR loops capable of binding albumin and non-CDR loops is obtained. Overlapping PCR is used to introduce random mutations in the non-CDR loop regions, thereby generating a library. The resultant sequences are cloned into a phage display vector, thereby generating a phage display library. Escherichia coli cells are transformed with the library and used to construct a phage display library. ELISA is performed using an immobilized target antigen binding domain with specificity for EGFR. A clone with high specificity for EGFR is selected. Affinity maturation is performed by re-randomizing residues in the non-CDR loop regions as before.

Sequence alignment of non-CDR loop regions of the resultant proteins is performed to determine sequence conservation between proteins with high affinity for the target antigen binding domain. Site directed mutagenesis of one or more amino acids within these regions of sequence conservation is performed to generate additional proteins. Binding of the resultant proteins to an immobilized target antigen binding domain whose target is EGFR is measured in an ELISA. A protein with the highest affinity for the target antigen binding domain is selected.

The sequence of this protein is cloned into a vector comprising a sequence for a cleavable linker. The resultant vector is expressed in a heterologous expression system to obtain a binding moiety comprising a cleavable linker and non-CDR loops which provide a binding site specific for a target antigen binding domain whose target is EGFR and CDR loops which are specific for IL-2.

### Example 2: Construction of an Exemplary Conditionally Active Chimeric Antigen Receptor

The binding moiety of Example 1 is cloned into an expression vector comprising the components of a chimeric antigen receptor, namely anti-PSMAscFv-IgG4-CD28tm-CD28costim-CD3zeta, to obtain a vector coding for a conditionally active chimeric antigen receptor. Primary human peripheral blood derived T-cells are activated and then lentivirally transduced with the conditionally active chimeric antigen receptor vector to obtain the conditionally active chimeric antigen receptor according to the present disclosure.

### Example 3: Construction of an Exemplary Conditionally Active T-Cell Receptor Fusion Protein

The binding moiety of Example 1 is cloned into an expression vector comprising the components of a T-cell receptor fusion protein, namely anti-PSMAscFv-IgG4-CD28tm-CD3epsilon, to obtain a vector coding for a conditionally active T-cell receptor fusion protein. Primary human peripheral blood derived T-cells are activated and then lentivirally transduced with the conditionally active T-cell receptor fusion protein vector to obtain the conditionally active T-cell receptor fusion protein according to the present disclosure.

### Example 4: Construction of an Exemplary Conditionally Active T-Cell Receptor

The binding moiety of Example 1 is cloned into an expression vector comprising the components of a T-cell receptor to obtain a vector coding for a conditionally active T-cell receptor. Primary human peripheral blood derived T-cells are activated and then lentivirally transduced with the conditionally active T-cell receptor vector to obtain the conditionally active T-cell receptor according to the present disclosure.

### Example 5: T-Cells Comprising a Conditionally Active Chimeric Antigen Receptor Exhibit Reduced Specificity Towards Cell Line which Overexpresses PSMA but is Protease Deficient

Cells overexpressing PSMA and exhibiting low expression of a matrix metalloprotease are separately incubated with T-cells comprising an exemplary conditionally active chimeric antigen receptor according to the present disclosure and T-cells comprising a non-conditionally active control chimeric antigen receptor. Cells expressing normal levels of PSMA and proteases are also incubated with T-cells comprising a conditionally active chimeric antigen receptor according to the present disclosure and T-cells comprising a non-conditionally active control chimeric antigen receptor. Both receptors comprise a target antigen binding domain with specificity toward PSMA.

Results indicate that in the absence of protease secretion, the T-cells comprising a conditionally active chimeric antigen receptor of the present disclosure interact with the protease expressing cells but do not interact with the PSMA expressed on the surface of the protease deficient cells. In contrast, the T-cells comprising a non-conditionally active control chimeric antigen receptor lack the ability to selectively bind the protease expressing cells over the protease deficient ones. Thus, the T-cells comprising an exemplary conditionally active chimeric antigen receptor of the present disclosure are advantageous, for example, in terms of reducing off-tumor toxicity.

### Example 6: T-Cells Comprising a Conditionally Active T-Cell Receptor Fusion Protein Exhibit Reduced Specificity Towards Cell Line which Overexpresses PSMA but is Protease Deficient

Cells overexpressing PSMA and exhibiting low expression of a matrix metalloprotease are separately incubated with T-cells comprising an exemplary conditionally active T-cell receptor fusion protein according to the present disclosure and T-cells comprising a non-conditionally active control T-cell receptor fusion protein. Cells expressing normal levels of PSMA and proteases are also incubated with T-cells comprising a conditionally active T-cell receptor fusion protein according to the present disclosure and T-cells comprising a non-conditionally active control T-cell receptor fusion protein. Both receptors comprise a target antigen binding domain with specificity toward PSMA.

Results indicate that in the absence of protease secretion, the T-cells comprising a conditionally active T-cell receptor fusion protein of the present disclosure interact with the protease expressing cells but do not interact with the PSMA expressed on the surface of the protease deficient cells. In contrast, the T-cells comprising a non-conditionally active control T-cell receptor fusion protein lack the ability to selectively bind the protease expressing cells over the protease deficient ones. Thus, the T-cells comprising an exemplary conditionally active T-cell receptor fusion protein of the present disclosure are advantageous, for example, in terms of reducing off-tumor toxicity.

### Example 7: T-Cells Comprising a Conditionally Active T-Cell Receptor Exhibit Reduced Specificity Towards Cell Line which Overexpresses PSMA but is Protease Deficient

Cells overexpressing PSMA and exhibiting low expression of a matrix metalloprotease are separately incubated with T-cells comprising an exemplary conditionally active T-cell receptor according to the present disclosure and T-cells comprising a non-conditionally active control T-cell receptor. Cells expressing normal levels of PSMA and proteases are also incubated with T-cells comprising a conditionally active T-cell receptor according to the present disclosure and T-cells comprising a non-conditionally active control T-cell receptor. Both receptors comprise a target antigen binding domain with specificity toward PSMA.

Results indicate that in the absence of protease secretion, the T-cells comprising a conditionally active T-cell receptor of the present disclosure interact with the protease expressing cells but do not interact with the PSMA expressed on the surface of the protease deficient cells. In contrast, the T-cells comprising a non-conditionally active control T-cell receptor lack the ability to selectively bind the protease expressing cells over the protease deficient ones. Thus, the T-cells comprising an exemplary conditionally active T-cell receptor of the present disclosure are advantageous, for example, in terms of reducing off-tumor toxicity.

### Example 8: Treatment with an Exemplary Conditionally Active Chimeric Antigen Receptor of the Present Disclosure Inhibits In Vivo Tumor Growth

Murine tumor line CT26 is implanted subcutaneously in Balb/c mice and on day 7 post-implantation the average size of the tumor is measured. Test mice are treated with T-cells comprising an exemplary conditionally active chimeric antigen receptor which has a target antigen binding domain specific for EGFR where in the EGFR-specific domain is bound to a binding moiety via its non-CDR loops and the binding moiety comprises a cleavable linker and CDR loops specific for IL-2. Control mice are treated with T-cells comprising a chimeric antigen receptor that contains a EGFR-specific domain but does not contain the binding moiety or the cleavable linker, and is not conditionally active. Results show that treatment with T-cells comprising an exemplary conditionally active chimeric antigen receptor of the present disclosure inhibits tumor more efficiently than the comparator T-cells comprising a chimeric antigen receptor which does not contain the moiety with non-CDR loops.

### Example 9: Treatment with an Exemplary Conditionally Active T-Cell Receptor Fusion Protein of the Present Disclosure Inhibits In Vivo Tumor Growth

Murine tumor line CT26 is implanted subcutaneously in Balb/c mice and on day 7 post-implantation the average size of the tumor is measured. Test mice are treated with T-cells comprising an exemplary conditionally active T-cell receptor fusion protein which has a target antigen binding domain specific for EGFR where in the EGFR-specific domain is bound to a binding moiety via its non-CDR loops and the binding moiety comprises a cleavable linker and CDR loops specific for IL-2. Control mice are treated with T-cells comprising a T-cell receptor fusion protein that contains a EGFR-specific domain but does not contain the binding moiety or the cleavable linker, and is not conditionally active. Results show that treatment with T-cells comprising an exemplary conditionally active T-cell receptor fusion protein of the present disclosure inhibits tumor more efficiently than the comparator T-cells comprising a T-cell receptor fusion protein which does not contain the moiety with non-CDR loops.

### Example 10: Treatment with an Exemplary Conditionally Active T-Cell Receptor of the Present Disclosure Inhibits In Vivo Tumor Growth

Murine tumor line CT26 is implanted subcutaneously in Balb/c mice and on day 7 post-implantation the average size of the tumor is measured. Test mice are treated with T-cells comprising an exemplary conditionally active T-cell receptor which has a target antigen binding domain specific for EGFR where in the EGFR-specific domain is bound to a binding moiety via its non-CDR loops and the binding moiety comprises a cleavable linker and CDR loops specific for IL-2. Control mice are treated with T-cells comprising a T-cell receptor that contains a EGFR-specific domain but does not contain the binding moiety or the cleavable linker, and is not conditionally active. Results show that treatment with the T-cells comprising an exemplary conditionally active T-cell receptor of the present disclosure inhibits tumor more efficiently than the comparator T-cells comprising a T-cell receptor which does not contain the moiety with non-CDR loops.

### Example 11: Conditionally Active Receptor Constructs

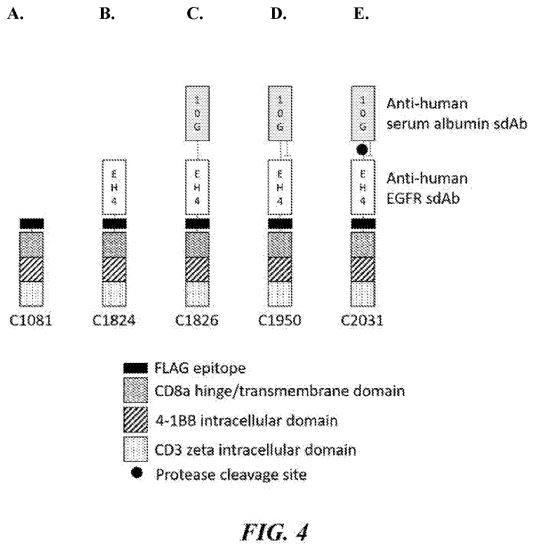

FIGS. 4A-F show examples of receptor constructs which were generated as disclosed herein. Each construct comprises an intracellular domain comprising a CD8 hinge/transmembrane domain (SEQ ID NO: 62), a 4-1BB intracellular domain (SEQ ID NO: 63), and a CD3 zeta intracellular domain (SEQ ID NO: 64). Construct C1081 (SEQ ID NO: 43; FIG. 4A) does not comprise a target antigen binding domain or a binding moiety. Construct C1824 (SEQ ID NO: 44; FIG. 4B) comprises the target antigen binding domain EH4 (SEQ ID NO: 60) but does not comprise a binding moiety. Construct 1826 (SEQ ID NO: 45; FIG. 4C) comprises target antigen binding domain EH4 and the binding moiety 10G with an unmodified non-CDR loop (SEQ ID NO: 56) along with a non-cleavable linker (SEQ ID NO: 65). Construct 1950 (SEQ ID NO: 46; FIG. 4D) comprises target antigen binding domain EH4 and binding moiety 10G with a modified non-CDR loop (SEQ ID NO: 57) along with a non-cleavable linker (SEQ ID NO: 65). Construct 2031 (SEQ ID NO: 47; FIG. 4E) comprises target antigen binding domain EH4 and binding moiety 10G with a modified non-CDR loop (SEQ ID NO: 57) along with protease cleavable linker 1 (SEQ ID NO: 66).

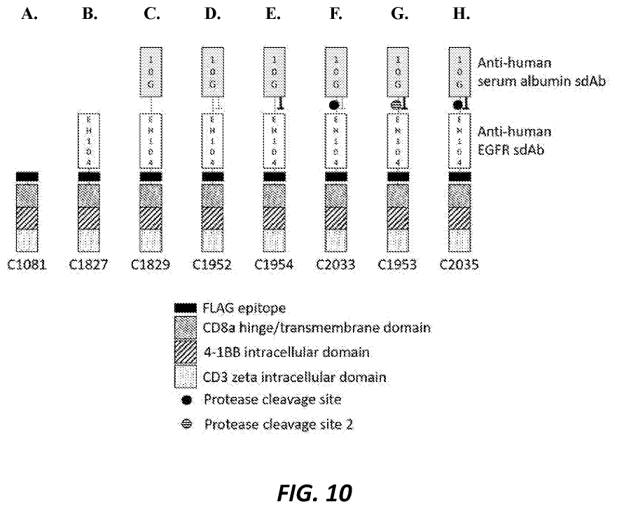

FIGS. 10A-H show examples of receptor constructs which were generated as disclosed herein. Construct C1081 (SEQ ID NO: 43; FIG. 10A) does not comprise a target antigen binding domain or a binding moiety. Construct 1827 (SEQ ID NO: 48; FIG. 10B) comprises the target antigen binding domain EH104 (SEQ ID NO: 61) but does not comprise a binding moiety. Construct 1829 (SEQ ID NO: 49; FIG. 10C) comprises target antigen binding domain EH104 and the binding moiety 10G with an unmodified non-CDR loop (SEQ ID NO: 56) along with a non-cleavable linker (SEQ ID NO: 65). Construct 1952 (SEQ ID NO: 50; FIG. 10D) comprises target antigen binding domain EH104 and binding moiety 10G with a modified non-CDR loop (SEQ ID NO: 58) along with a non-cleavable linker (SEQ ID NO: 65). Construct 1954 (SEQ ID NO: 51; FIG. 10E) comprises target antigen binding domain EH104 and binding moiety 10G with a modified non-CDR loop (SEQ ID NO: 59) along with a non-cleavable linker (SEQ ID NO: 65). Construct 2033 (SEQ ID NO: 52; FIG. 10F) comprises target antigen binding domain EH104 and binding moiety 10G with a modified non-CDR loop (SEQ ID NO: 58) along with protease cleavable linker 1 (SEQ ID NO: 66). Construct 1953 (SEQ ID NO: 53; FIG. 10G) comprises target antigen binding domain EH104 and binding moiety 10G with a modified non-CDR loop (SEQ ID NO: 59) along with protease cleavable linker 2 (SEQ ID NO: 67). Construct 2035 (SEQ ID NO: 54; FIG. 10H) comprises target antigen binding domain EH104 and binding moiety 10G with a modified non-CDR loop (SEQ ID NO: 59) along with protease cleavable linker 1 (SEQ ID NO: 66).

### Example 12: Inhibition of CAR-T Cell Killing Activity by Modified Non-CDR Loop Mask

Chimeric antigen receptor-expressing T-cells (CAR T-cells) were generated by infecting isolated CD4/CD8 positive T cells from a healthy donor with lentivirus expressing the indicated constructs. FIG. 4 shows a diagram with the constructs used. CAR T-cells were mixed at a 10:1 ratio with EGFR-expressing cancer cells HCT116, engineered to express luciferase. After 72 hours, luciferase activity was measured as a readout of HCT116 viability. CAR-T cells that lacked a target antigen binding domain (C1081) were used as a negative control. The assay was performed with or without human serum albumin (HSA), as indicated.

The results of this experiment are shown in FIG. 5. These results indicate that HSA has weak steric blocking activity of CAR T-cell killing activity. These results also demonstrate that the construct containing binding moiety 10G with a modified non-CDR loop (C1950) significantly blocks cell killing activity.

### Example 13: Chimeric Antigen Receptor Binding to EGFR

Chimeric antigen receptor-expressing T-cells (CAR T-cells) were generated by infecting isolated CD4/CD8 positive T cells from a healthy donor with lentivirus expressing the indicated constructs. FIG. 4 shows a diagram with the constructs used. Cells were incubated with Fc-tagged EGFR extracellular domain (ECD). Cells were washed to remove unbound Fc-tagged EGFR ECD. Cells were incubated with a secondary antibody conjugated to DyLight 650 that recognizes human Fc. Binding of the secondary antibody to cells was measured by flow cytometry.

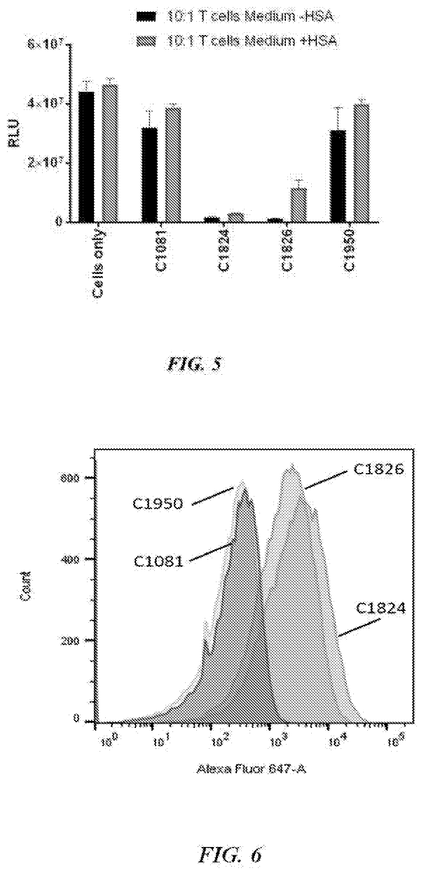

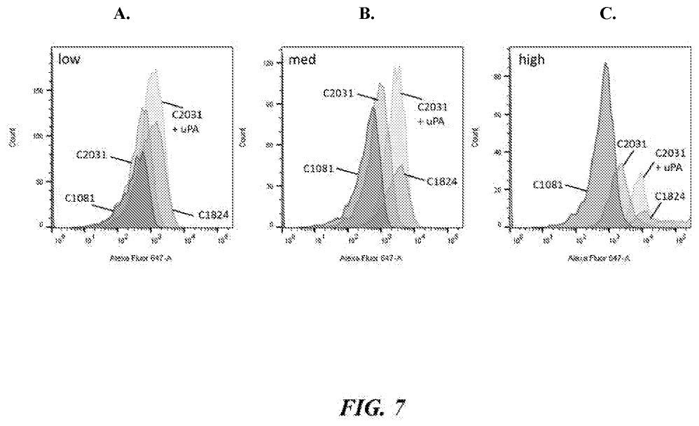

The results of this experiment are shown in FIG. 6. Masked EH4 ProCAR (C1950, comprising binding moiety 10G with a modified non-CDR loop) demonstrated no detectable binding to its antigen EGFR-Fc while the positive controls (C1824, construct comprising the binding domain EH4 but devoid of binding moiety 10G) bound strongly.

The construct C1826, comprising the EGFR binding domain EH4 and the anti-ALB binding moiety 10G with an unmodified non-CDR loop and a non-cleavable linker, i.e., without masking of EH4 binding domain, shows very little steric blocking. This indicates that the anti-ALB binding domain on its own without the mask shows very little steric blocking and that the blocking of EGFR binding, seen in case of C1950, is an effect of the masking.

### Example 14: Protease Activation of Conditionally Active Chimeric Antigen Receptors to EGFR

Chimeric antigen receptor-expressing T-cells (CAR T-cells) were generated by infecting isolated CD4/CD8 positive T cells from a healthy donor with lentivirus expressing the indicated constructs. FIG. 4 shows a diagram with the constructs used. CAR T-cells expressing C2031 were treated with either PBS or recombinant uPA protease, as indicated, for 1 hour at room temperature. All other samples were treated with PBS for 1 hour at room temp. Cells were incubated with Fc-tagged EGFR extracellular domain (ECD) and mouse anti-FLAG antibody. Cells were washed to remove unbound Fc-tagged EGFR ECD and anti-FLAG antibody. Cells were incubated with a secondary antibody conjugated to DyLight 650 that recognizes human Fc and a secondary antibody conjugated to BV421 that recognizes mouse antibodies. Binding of the secondary antibodies to cells was measured by flow cytometry. Samples were analyzed using FLOWJO® software to bin CAR T-cells into low, medium, and high expression groups based on anti-FLAG/BV421 staining. This served to normalize expression between different constructs.

The results of this experiment are shown in FIG. 7. Treatment of the cells expressing the conditionally activated chimeric antigen receptor, C2031, with uPA activated EGFR-binding activity to the same level as the positive control cells expressing C1824.

### Example 15: Protease Cleavage Site-Dependent Activation of CAR T-Cell Activity

Chimeric antigen receptor-expressing T-cells (CAR T-cells) were generated by infecting isolated CD4/CD8 positive T cells from a healthy donor with lentivirus expressing the indicated constructs. FIG. 4 shows a diagram with the constructs used. CAR T-cells were mixed at a 10:1 ratio with EGFR-expressing cancer cells HCT116, engineered to express luciferase. After 72 hours, luciferase activity was measured as a readout of HCT116 viability. CAR-T cells that lacked a target antigen binding domain (C1081) were used as a negative control.

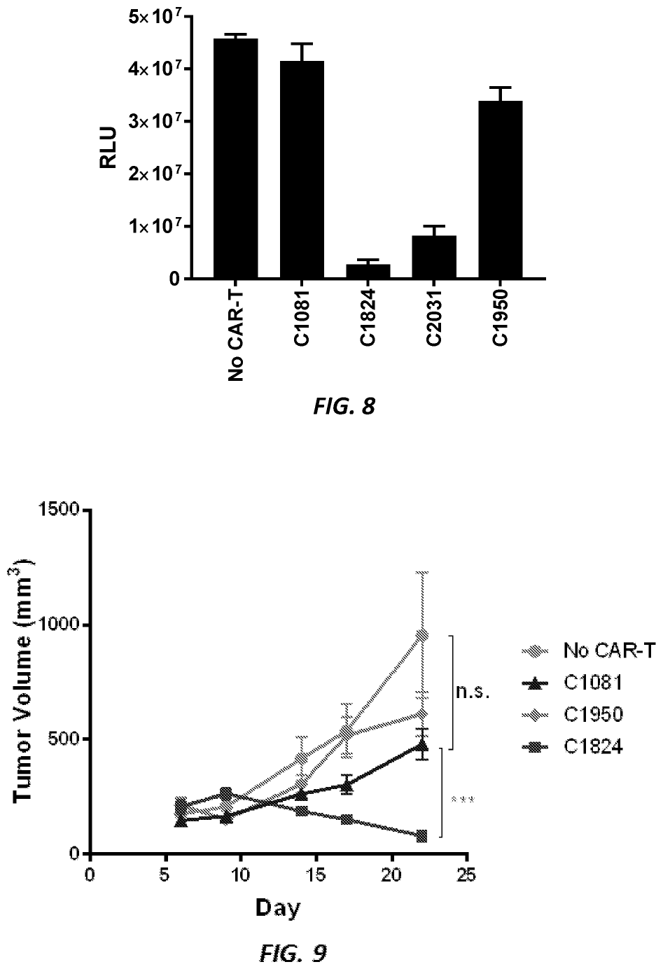

The results of this experiment are shown in FIG. 8. The CAR T-cell expressing a receptor without a protease cleavage site (C1950) showed little cell killing activity. The CAR T-cell expressing a receptor with a protease cleavage site (C2031) showed significant cell killing activity.

### Example 16: In Vivo Masking with Non-Cleavable ProCAR Constructs

NSG mice were subcutaneously implanted with an admixture of HCT116 cancer cells and the indicated CAR-T cells from FIG. 4 at a ratio of 1:1. Tumor volume was measured at the indicated days post-implantation. The results of this experiment are show in FIG. 9. While the CAR-T expressing a receptor with only a target binding domain (C1824) was able to inhibit tumor formation, the CAR-T cell expressing a receptor with a binding moiety containing a modified non-CDR loop and a noncleavable protease linker (C1950) had no activity. Significance was determined by using unpaired t test between groups. *** denotes p<0.0005.

### Example 17: Inhibition of CAR-T Cell Killing Activity by Modified Non-CDR Loop Mask

Chimeric antigen receptor-expressing T-cells (CAR T-cells) were generated by infecting isolated CD4/CD8 positive T cells from a healthy donor with lentivirus expressing the indicated constructs. FIG. 10 shows a diagram with the constructs used. CAR T-cells were mixed at a 10:1 ratio with EGFR-expressing cancer cells HCT116, engineered to express luciferase. After 72 hours, luciferase activity was measured as a readout of HCT116 viability. CAR-T cells that lacked a target antigen binding domain (C1081) were used as a negative control.

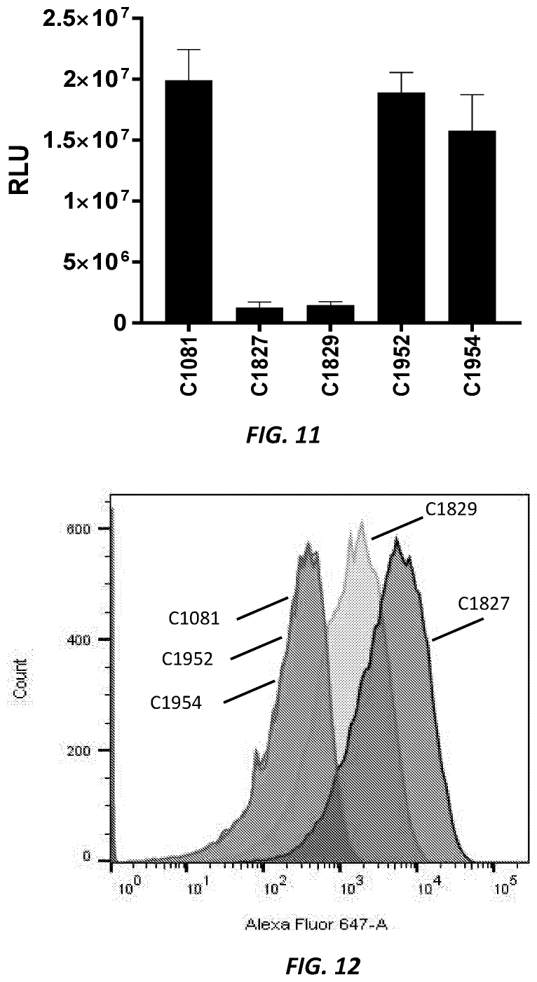

The results of this experiment are shown in FIG. 11. These results demonstrate that the construct containing binding domain 10G with a modified non-CDR loop (C1952 and 1954) significantly blocks cell killing activity. The construct containing binding domain 10G without a modified non-CDR loop (C1829) did not block cell killing activity.

### Example 18: Chimeric Antigen Receptor Binding to EGFR

Chimeric antigen receptor-expressing T-cells (CAR T-cells) were generated by infecting isolated CD4/CD8 positive T cells from a healthy donor with lentivirus expressing the indicated constructs. FIG. 10 shows a diagram with the constructs used. Cells were incubated with Fc-tagged EGFR extracellular domain (ECD). Cells were washed to remove unbound Fc-tagged EGFR ECD. Cells were incubated with a secondary antibody conjugated to DyLight 650 that recognizes human Fc. Binding of the secondary antibody to cells was measured by flow cytometry.

The results of this experiment are shown in FIG. 12. C1954 and C1952, each comprising binding domain 10G with a unique modified non-CDR loop, show no detectable binding to EGFR-Fc, while positive control C1827 (construct comprising the target antigen binding domain EH104 but no binding moiety) binds strongly.

The construct C1829, comprising the EGFR binding domain EH104 and the anti-ALB binding moiety 10G with an unmodified non-CDR loop and a non-cleavable linker, i.e., without masking of EH4 binding domain, shows very little steric blocking. This indicates that the anti-ALB binding domain on its own without the mask shows very little steric blocking and that the blocking of EGFR binding, seen in case of C1954 and C1952, is an effect of the masking.

### Example 19: Protease Activation of Conditionally Active Chimeric Antigen Receptors to EGFR

Chimeric antigen receptor-expressing T-cells (CAR T-cells) were generated by infecting isolated CD4/CD8 positive T cells from a healthy donor with lentivirus expressing the indicated constructs. FIG. 10 shows a diagram with the constructs used. CAR T-cells expressing C2033 were treated with either PBS or recombinant uPA protease, as indicated, for 1 hour at room temperature. All other samples were treated with PBS for 1 hour at room temp. Cells were incubated with Fc-tagged EGFR extracellular domain (ECD) and mouse anti-FLAG antibody. Cells were washed to remove unbound Fc-tagged EGFR ECD and anti-FLAG antibody. Cells were incubated with a secondary antibody conjugated to DyLight 650 that recognizes human Fc and a secondary antibody conjugated to BV421 that recognizes mouse antibodies. Binding of the secondary antibodies to cells was measured by flow cytometry. Samples were analyzed using FLOWJO® software to bin CAR T-cells into low, medium, and high expression groups based on anti-FLAG/BV421 staining. This served to normalize expression between different constructs.

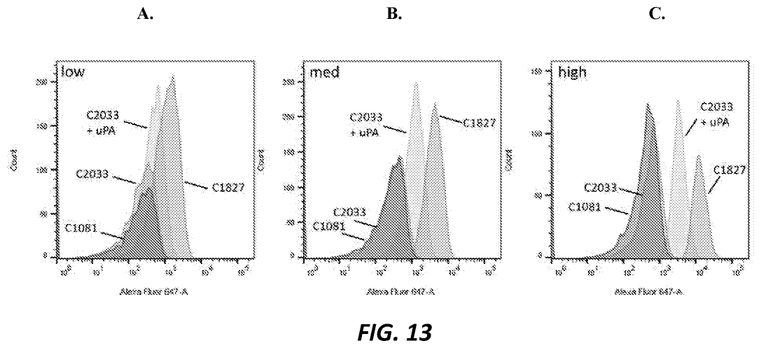

The results of this experiment are shown in FIG. 13. Treatment of C2033 expressing cells with uPA activated EGFR-binding activity.

### Example 20: Protease Activation of Conditionally Active Chimeric Antigen Receptors to EGFR

Chimeric antigen receptor-expressing T-cells (CAR T-cells) were generated by infecting isolated CD4/CD8 positive T cells from a healthy donor with lentivirus expressing the indicated constructs. FIG. 10 shows a diagram with the constructs used. CAR T-cells expressing C2035 were treated with either PBS or recombinant uPA protease, as indicated, for 1 hour at room temperature. All other samples were treated with PBS for 1 hour at room temp. Cells were incubated with Fc-tagged EGFR extracellular domain (ECD) and mouse anti-FLAG antibody. Cells were washed to remove unbound Fc-tagged EGFR ECD and anti-FLAG antibody. Cells were incubated with a secondary antibody conjugated to DyLight 650 that recognizes human Fc and a secondary antibody conjugated to BV421 that recognizes mouse antibodies. Binding of the secondary antibodies to cells was measured by flow cytometry. Samples were analyzed using FLOWJO® software to bin CAR T-cells into low, medium, and high expression groups based on anti-FLAG/BV421 staining. This served to normalize expression between different constructs.

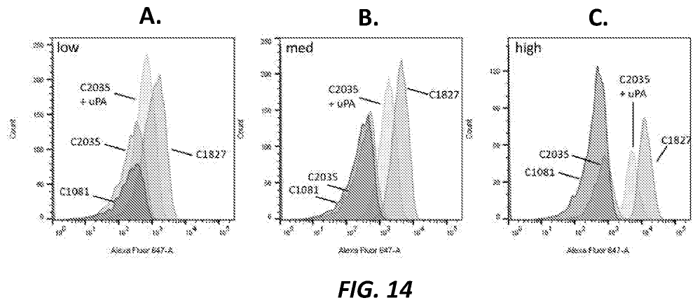

The results of this experiment are shown in FIG. 14. Treatment of C2035 expressing cells with uPA activated EGFR-binding activity.

### Example 21: Protease Cleavage Site-Dependent Activation of CAR T-Cell Activity

Chimeric antigen receptor-expressing T-cells (CAR T-cells) were generated by infecting isolated CD4/CD8 positive T cells from a healthy donor with lentivirus expressing the indicated constructs. FIG. 10 shows a diagram with the constructs used. CAR T-cells were mixed at a 10:1 ratio with EGFR-expressing cancer cells HCT116, engineered to express luciferase. After 72 hours, luciferase activity was measured as a readout of HCT116 viability. CAR-T cells that lacked a target antigen binding domain (C1081) were used as a negative control.

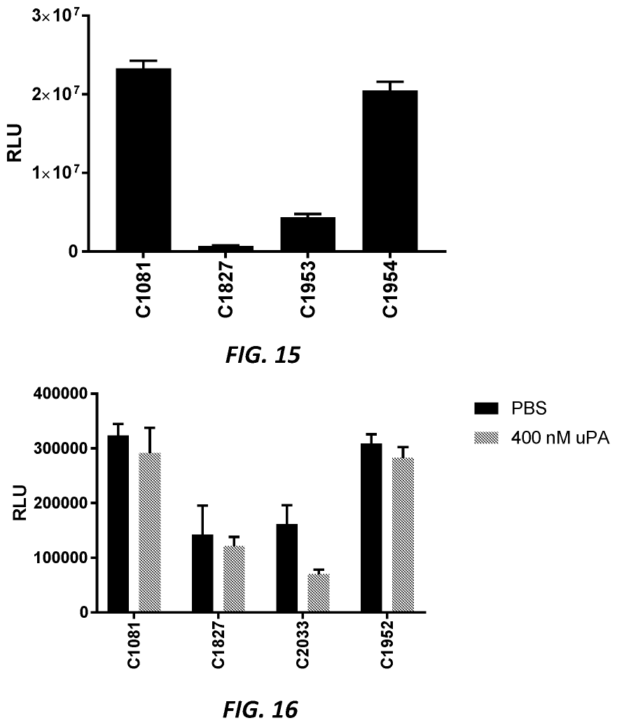

The results of this experiment are shown in FIG. 15. The CAR T-cell expressing a receptor without a protease cleavage site (C1954) showed little cell killing activity. The CAR T-cell expressing a receptor with a protease cleavage site (C1953) showed significant cell killing activity.

### Example 22: Protease Activation of CAR T-Cell Killing Activity

Chimeric antigen receptor-expressing T-cells (CAR T-cells) were generated by infecting isolated CD4/CD8 positive T cells from a healthy donor with lentivirus expressing the indicated constructs. FIG. 10 shows a diagram with the constructs used. CAR-T cells were washed once with PBS and then treated with either PBS or PBS containing 400 nM recombinant uPA protease for 1 hour at room temperature. CAR-T cells were then mixed at a 40:1 ratio with EGFR-expressing HCT116 cells engineered to express luciferase. After 24 hours luciferase activity was measured as a readout of HCT116 viability. CAR-T cells that lacked a target antigen binding domain (C1081) were used as a negative control.

The results of this experiment are shown in FIG. 16. Cells expressing the non-cleavable receptor C1952 showed little cell killing activity. Cells expressing the cleavable receptor C2033 showed moderate cell killing, which was further increased by treatment with recombinant uPA protease.

### Example 22: Protease Activation of CAR T-Cell Killing Activity

Chimeric antigen receptor-expressing T-cells (CAR T-cells) were generated by infecting isolated CD4/CD8 positive T cells from a healthy donor with lentivirus expressing the indicated constructs. FIG. 10 shows a diagram with the constructs used. CAR-T cells were then mixed at a 10:1 ratio with HCT116 cells engineered to express luciferase in T cell culture media with or without 100 nM recombinant Matriptase protease. After 72 hours luciferase activity was measured as a readout of HCT116 viability. CAR-T cells that lacked a target antigen binding domain (C1081) were used as a negative control.

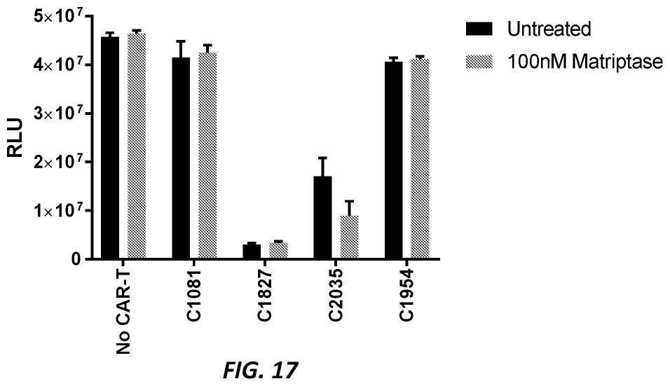

The results of this experiment are shown in FIG. 17. Cells expressing the non-cleavable receptor C1954 showed little cell killing activity. Cells expressing the cleavable receptor C2035 showed moderate cell killing activity, which was further increased by treatment with recombinant Matriptase protease.

### Example 23: Construction and Testing of Exemplary Multivalent Target Binding Proteins

Constructs

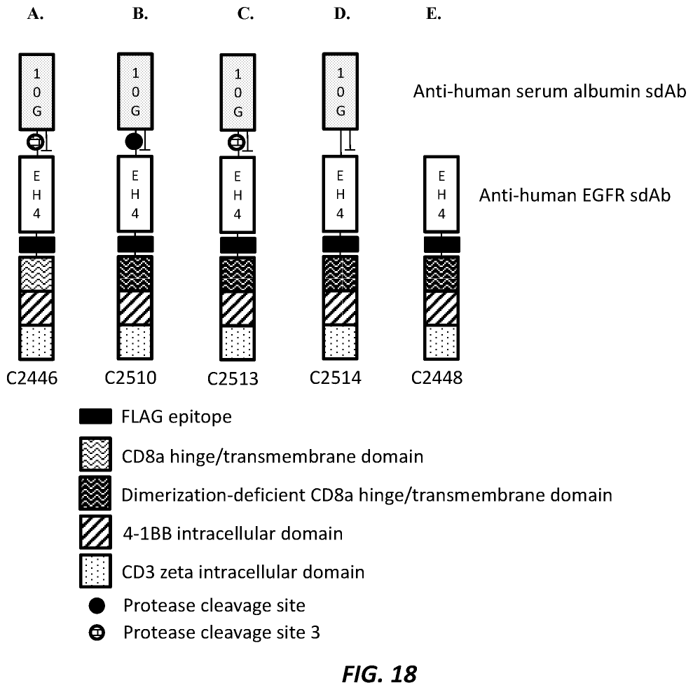

The following constructs were made. Construct C2446 includes an anti-human serine albumin sdAb, a protease cleavage site 3, an anti-human EGFR sdAb, a FLAG epitope, a CD8 hinge/transmembrane domain, a 4-1BB intracellular domain, and a CD3 zeta intracellular domain (FIG. 18A; SEQ ID NO: 71). Construct C2510 includes an anti-human serine albumin sdAb, a protease cleavage site, an anti-human EGFR sdAb, a FLAG epitope, a dimerization-deficient CD8a hinge/transmembrane domain, a 4-1BB intracellular domain, and a CD3 zeta intracellular domain (FIG. 18B; SEQ ID NO: 73). Construct C2513 includes an anti-human serine albumin sdAb, a protease cleavage site 3, an anti-human EGFR sdAb, a FLAG epitope, a dimerization-deficient CD8a hinge/transmembrane domain, a 4-1BB intracellular domain, and a CD3 zeta intracellular domain (FIG. 18C; SEQ ID NO: 74).Construct C2514 includes an anti-human serine albumin sdAb, an anti-human EGFR sdAb, a FLAG epitope, a dimerization-deficient CD8a hinge/transmembrane domain, a 4-1BB intracellular domain, and a CD3 zeta intracellular domain (FIG. 18D; SEQ ID NO: 75). Construct C2448, includes an anti-human EGFR sdAb, a FLAG epitope, a dimerization-deficient CD8a hinge/transmembrane domain, a 4-1BB intracellular domain, and a CD3 zeta intracellular domain (FIG. 18E; SEQ ID NO: 72).

In Vitro Protease Site-Dependent Cell Killing Activity

In a first assay, 300,000 primary human T cells isolated from healthy donors were infected with 1 mL lentiviral supernatant made from the indicated constructs to generate anti-EGFR CAR-T cells. Twenty five thousand CAR-T cells were subsequently co-cultured under standard conditions at a 10:1 ratio (CAR-T:Target cells) with EGFR-expressing cancer cells that stably express luciferase for 72 hours. Luciferase activity is determined as a proxy for cancer cell viability and normalized to the control CAR-T cells that do not contain scFv.

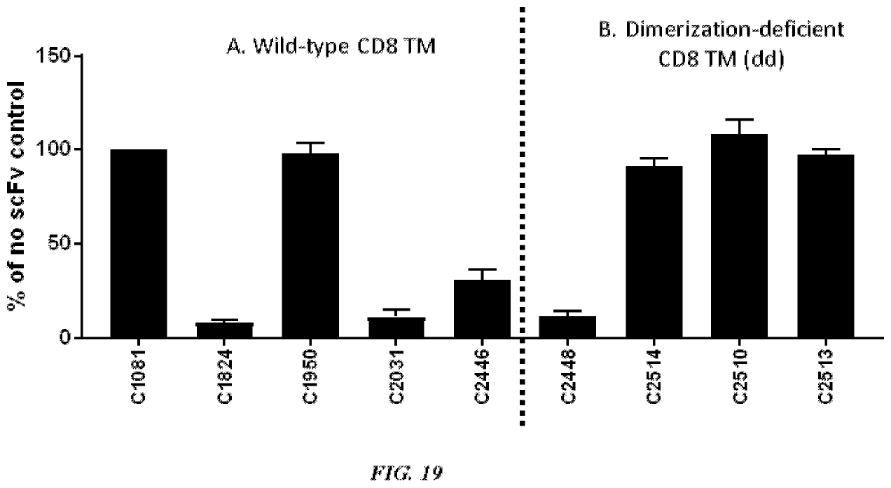

The data demonstrated that the level of protease side-dependent cell killing activity was reduced in ProCAR constructs that lacked the dimerization activity of the CD8a transmembrane domain (FIG. 19B) compared to wild-type (FIG. 19A). The dimerization mutation reduces the amount of cell killing activity in the absence of exogenous protease (e.g., compare C2031 to C2510, or compare C2446 to C2513).

In a second assay, 300,000 primary human T cells isolated from healthy donors were infected with 1 mL lentiviral supernatant made from the indicated constructs to generate anti-EGFR CAR-T cells, which were subsequently co-cultured at ratios of 10:1, 5:1, 2.5:1, and 1:1 (CAR-T:Target cells) with EGFR-expressing cancer cells that stably express luciferase for 72 hours. Luciferase activity is determined as a proxy for cancer cell viability and normalized to the control CAR-T cells that do not contain scFv.

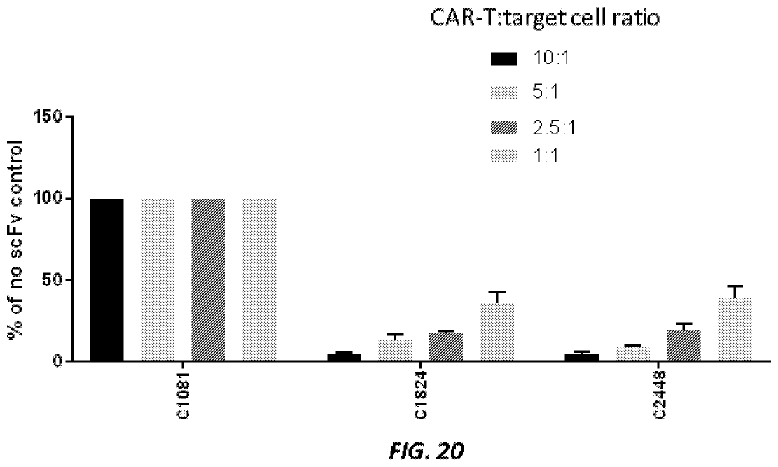

The data demonstrates that dimerization-deficient EGFR CAR has similar killing activity compared to wild-type (FIG. 20).

### Example 24: Construction and Testing of Exemplary Multivalent Target Binding Proteins

Constructs

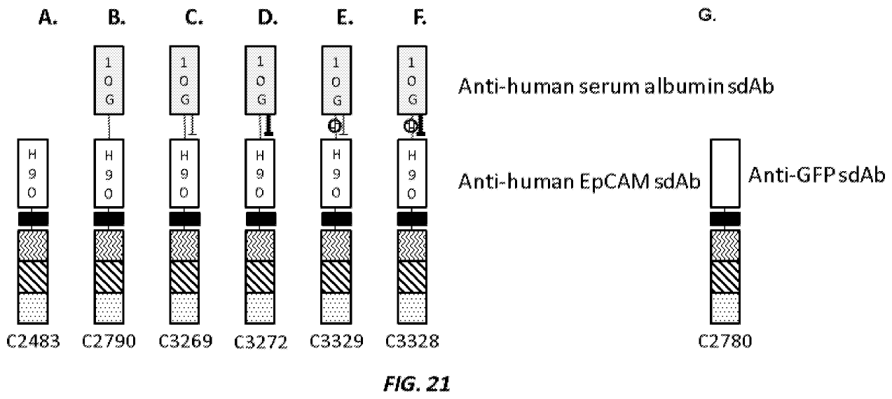

The following ProCAR constructs were made. Construct C2483 includes an anti-human EpCAM sdAb, a FLAG epitope, a CD8 hinge/transmembrane domain, a 4-1BB intracellular domain, and a CD3 zeta intracellular domain (FIG. 21A; SEQ ID NO: 77). Construct C2790 includes an anti-human serum albumin sdAb, an anti-human EpCAM sdAb, a FLAG epitope, a CD8 hinge/transmembrane domain, a 4-1BB intracellular domain, and a CD3 zeta intracellular domain (FIG. 21B; SEQ ID NO: 78).Construct C3269 includes an anti-human serum albumin sdAb, an anti-human EpCAM sdAb, a FLAG epitope, a CD8 hinge/transmembrane domain, a 4-1BB intracellular domain, and a CD3 zeta intracellular domain (FIG. 21C; SEQ ID NO: 79). Construct C3272 includes an anti-human serum albumin sdAb, an anti-human EpCAM sdAb, a FLAG epitope, a CD8 hinge/transmembrane domain, a 4-1BB intracellular domain, and a CD3 zeta intracellular domain (FIG. 21D; SEQ ID NO: 80). Construct C3329 includes an anti-human serum albumin sdAb, a protease cleavage site 3, an anti-human EpCAM sdAb, a FLAG epitope, a CD8 hinge/transmembrane domain, a 4-1BB intracellular domain, and a CD3 zeta intracellular domain (FIG. 21E; SEQ ID NO: 82). Construct C3328 includes an anti-human serum albumin sdAb, a protease cleavage site 3, an anti-human EpCAM sdAb, a FLAG epitope, a CD8 hinge/transmembrane domain, a 4-1BB intracellular domain, and a CD3 zeta intracellular domain (FIG. 21F; SEQ ID NO: 81). Construct C2780 includes an anti-GFP sdAb, a FLAG epitope, a CD8 hinge/transmembrane domain, a 4-1BB intracellular domain, and a CD3 zeta intracellular domain (FIG. 21G; SEQ ID NO: 76).

EpCAM Mask 1 Blocks ProCAR EpCAM-Binding Activity

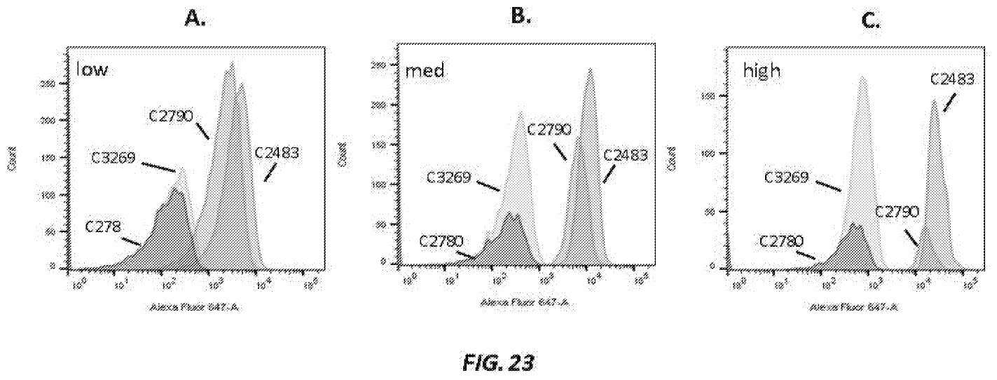

300,000 primary human T cells isolated from healthy donors were infected with 1 mL lentiviral supernatant made from the indicated constructs from FIG. 21 to generate anti-EpCAM CAR-T cells, which were subsequently stained with anti-FLAG antibodies and EGFR-Fc along with the indicated secondary antibodies. The data was analyzed by flow cytometry. FIG. 23 provides histograms of EpCAM-Fc/Alexa Fluor 647 staining of CAR-T cells that have been grouped into low (FIG. 23A), medium (FIG. 23B), or high (FIG. 23C) CAR expression based on anti-FLAG staining.

This data demonstrates the efficacy of EpCAM mask 1 blocking ProCAR EpCAM-binding activity.

EpCAM Mask 2 Blocks ProCAR EpCAM-Binding Activity

300,000 primary human T cells isolated from healthy donors were infected with 1 mL lentiviral supernatant made from the indicated constructs from FIG. 21 to generate anti-EpCAM CAR-T cells, which were subsequently stained with anti-FLAG antibodies and EGFR-Fc along with the indicated secondary antibodies. The data was analyzed by flow cytometry.

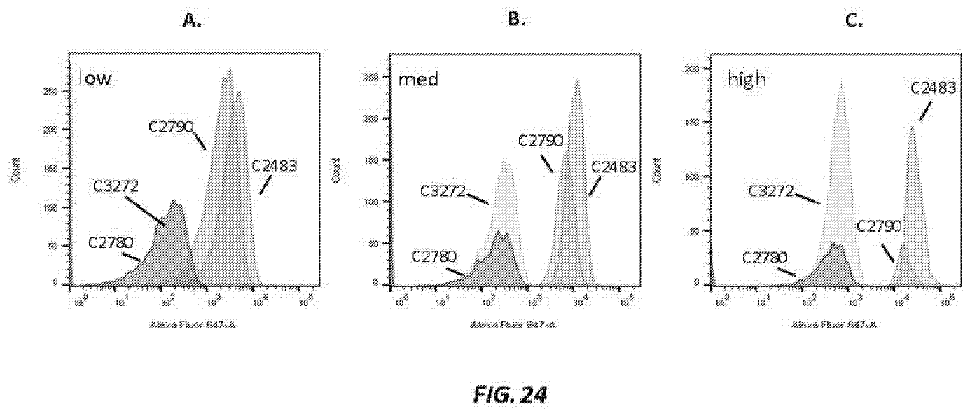

FIG. 24 provides histograms of EpCAM-Fc/Alexa Fluor 647 staining of CAR-T cells from FIG. 21 that have been grouped into low (FIG. 24A), medium (FIG. 24B), or high (FIG. 24C) CAR expression based on anti-FLAG staining that demonstrate the efficacy of EpCAM mask 2 in blocking ProCAR EpCAM-binding activity.

Masking of Anti-EpCAM sdAb H90

300,000 primary human T cells isolated from healthy donors were infected with 1 mL lentiviral supernatant made from the indicated constructs from FIG. 21 to generate anti-EpCAM CAR-T cells, which were subsequently co-cultured at various ratios (CAR-T:Target cells) with EGFR-expressing cancer cells that stably express luciferase. Luciferase activity was read 72 hours later as a proxy for cancer cell viability and normalized to the anti-GFP control CAR-T cells, C2780.

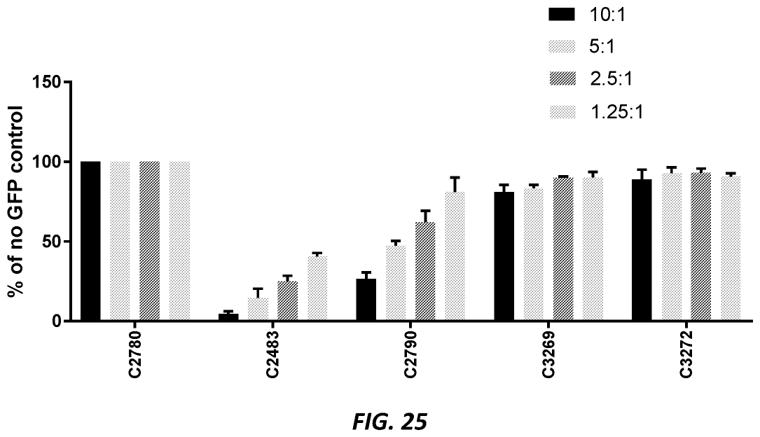

The data provided in FIG. 25 demonstrates masking of the anti-EpCAM sdAb H90.

C2843 is the “naked” CAR, i.e., no anti-ALB domain. Addition of the anti-ALB domain (C2790) has a small impact on cell killing activity. Addition of a mask to the CC′ non-CDR loop of the anti-ALB domain has a large impact on cell killing activity due to specific blocking of EpCAM binding.

Steric Blocking by HSA of AntiEpCAM sdAb H90

300,000 primary human T cells isolated from healthy donors were infected with 1 mL lentiviral supernatant made from the indicated constructs from FIG. 21 to generate anti-EpCAM CAR-T cells, which were subsequently co-cultured at various ratios (CAR-T:Target cells) with EGFR-expressing cancer cells that stably express luciferase in the presence or absence of human serum albumin (HSA). Luciferase activity was read 72 hours later as a proxy for cancer cell viability and normalized to the anti-GFP control CAR-T cells, C2780.

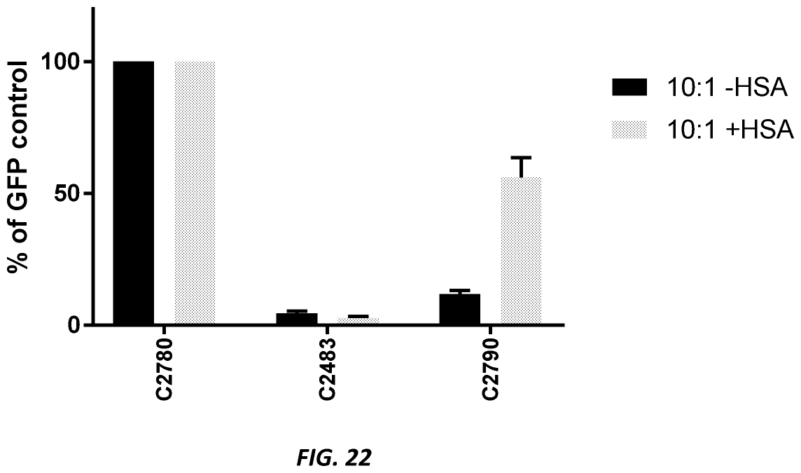

The data provided in FIG. 22 demonstrate steric blocking by HSA of the anti-EpCAM sdAb H90.

### Example 25: EpCAM ProCAR Protease Site-Dependent Cell Killing

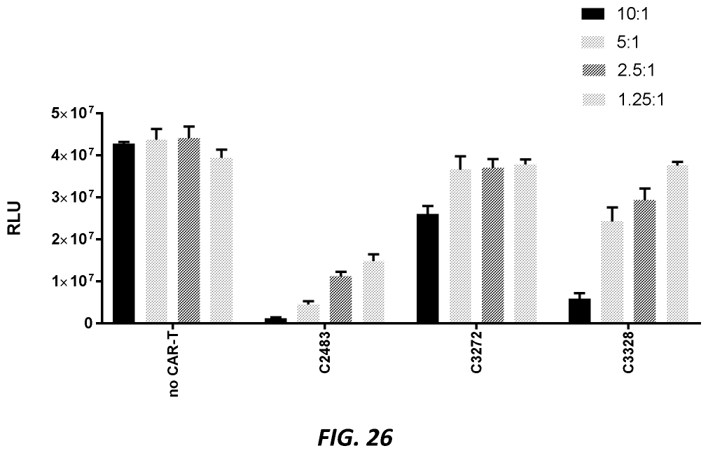

300,000 primary human T cells isolated from healthy donors were infected with 1 mL lentiviral supernatant made from the indicated constructs from FIG. 21 to generate anti-EpCAM CAR-T cells, which were subsequently co-cultured at various ratios (CAR-T:Target cells) with EGFR-expressing cancer cells that stably express luciferase. Luciferase activity was read 72 hours later as a proxy for cancer cell viability and normalized to the anti-GFP control CAR-T cells, C2780 FIG. 26 demonstrates protease site-dependent cell killing activity of CAR-T cells expressing a masked EpCAM ProCAR.

### Example 26: Protease Activation of EpCAM Mask 1 ProCAR Antigen Binding Activity

300,000 primary human T cells isolated from healthy donors were infected with 1 mL lentiviral supernatant made from the indicated constructs from FIG. 21 to generate anti-EpCAM CAR-T cells, which were subsequently washed in PBS, and then treated for 1 hr at room temp with either PBS or PBS containing 400 nM recombinant uPA protease and then stained with anti-FLAG antibodies and EpCAM-Fc along with the anti-mouse BV421 and anti-human Fc Alexa Fluor 647 secondary antibodies and analyzed by flow cytometry.

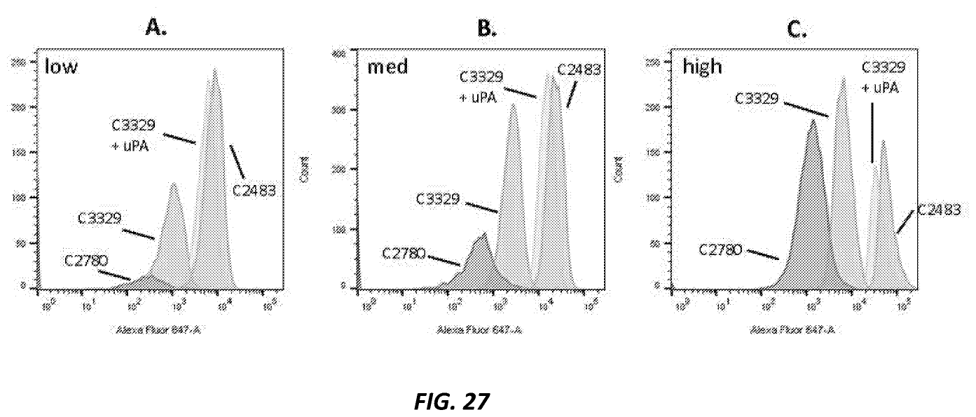

Histograms of EpCAM-Fc staining of CAR-T cells that have been grouped into low (FIG. 27A), medium (FIG. 27B), or high (FIG. 27C) CAR expression based on anti-FLAG staining. These results demonstrate protease activation of EpCAM Mask 1 ProCAR antigen binding activity.

### Example 26: Protease Activation of EpCAM Mask 2 ProCAR Antigen Binding Activity

300,000 primary human T cells isolated from healthy donors were infected with 1 mL lentiviral supernatant made from the indicated constructs from FIG. 21 to generate anti-EpCAM CAR-T cells, which were subsequently washed in PBS, and then treated for 1 hr at room temp with either PBS or PBS containing 400 nM recombinant uPA protease and then stained with anti-FLAG antibodies and EpCAM-Fc along with the anti-mouse BV421 and anti-human Fc Alexa Fluor 647 secondary antibodies and analyzed by flow cytometry.

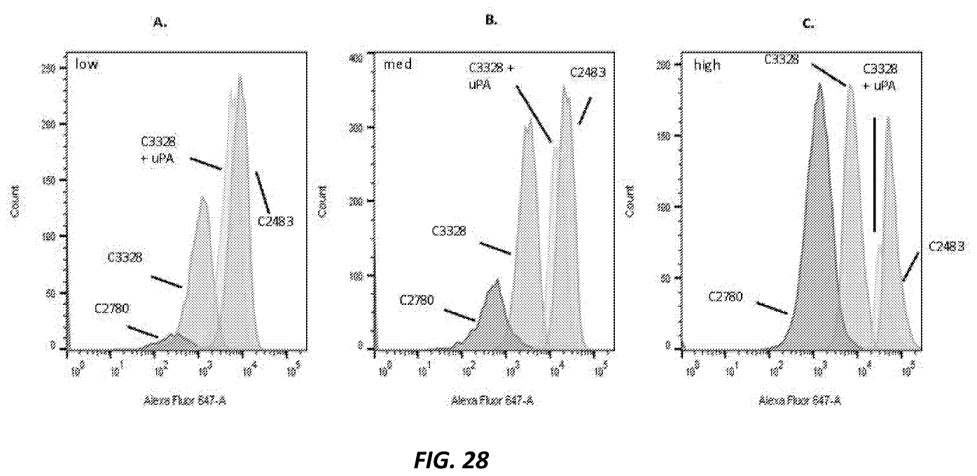

Histograms of EpCAM-Fc staining of CAR-T cells that have been grouped into low (FIG. 28A), medium (FIG. 28B), or high (FIG. 28C) CAR expression based on anti-FLAG staining. These results demonstrate protease activation of EpCAM Mask 2 ProCAR antigen binding activity.

SEQUENCE LISTING

| SEQ | | |
|---|---|---|
| ID | | |
| NO: | Description | Sequence |
| 43. | C1081 | MALPVTALLLPLALLLHAARPTSDYKDDDDKTTTPAPRPPTPAPTIASQ |
| | | PLSLRPEACRPAAGGAVHTRGLDFACDIYIWAPLAGTCGVLLLSLVITL |
| | | YKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPR |
| | | RKNPQEGLYNELQKDKMAEAYSEIGMKGERRRGKGHDGLYQGLSTA |
| | | TKDTYDALHMQALPPR |
| 44. | C1824 | MALPVTALLLPLALLLHAARPEVQLLESGGGLVQPGGSLTLSCAASGR |
| | | SFSNYIMGWFRQAPGKEREFVAGLGWSPGNTYYADSVKGRFTISRD |
| | | NSKNTLYLQMNSLRAEDTAVYYCAARRGDVIYTTPWNYVYWGLGTQ |
| | | VTVSSTSDYKDDDDKTTTPAPRPPTPAPTIASQPLSLRPEACRPAAGG |
| | | AVHTRGLDFACDIYIWAPLAGTCGVLLLSLVITLYKRGRKKLLYIFKQPF |
| | | MRPVQTTQEEDGCSCRFPEEEEGGCELRVKFSRSADAPAYQQGQNQ |
| | | LYNELNLGRREEYDVLDKRRGRDPEMGGKPRRKNPQEGLYNELQKD |
| | | KMAEAYSEIGMKGERRRGKGHDGLYQGLSTATKDTYDALHMQALPP |
| | | R |
| 45. | C1826 | MALPVTALLLPLALLLHAARPEVQLVESGGGLVQPGNSLRLSCAASGF |
| | | TFSKFGMSWVRQAPGKGLEWVSSISGSGRDTLYADSVKGRFTISRDN |
| | | AKTTLYLQMNSLRPEDTAVYYCTIGGSLSVSSQGTLVTVSSGSGGSGG |
| | | GGSGGGGGSGEVQLLESGGGLVQPGGSLTLSCAASGRSFSNYIMGW |
| | | FRQAPGKEREFVAGLGWSPGNTYYADSVKGRFTISRDNSKNTLYLQM |
| | | NSLRAEDTAVYYCAARRGDVIYTTPWNYVYWGLGTQVTVSSTSDYKD |
| | | DDDKTTTPAPRPPTPAPTIASQPLSLRPEACRPAAGGAVHTRGLDFAC |
| | | DIYIWAPLAGTCGVLLLSLVITLYKRGRKKLLYIFKQPFMRPVQTTQEED |
| | | GCSCRFPEEEEGGCELRVKFSRSADAPAYQQGQNQLYNELNLGRREE |
| | | YDVLDKRRGRDPEMGGKPRRKNPQEGLYNELQKDKMAEAYSEIGM |
| | | KGERRRGKGHDGLYQGLSTATKDTYDALHMQALPPR |
| 46. | C1950 | MALPVTALLLPLALLLHAARPEVQLVESGGGLVQPGNSLRLSCAASGF |
| | | TFSKFGMSWVRQANPWVPPREGLEWVSSISGSGRDTLYADSVKGRF |
| | | TISRDNAKTTLYLQMNSLRPEDTAVYYCTIGGSLSVSSQGTLVTVSSGS |
| | | GGSGGGGSGGGGGSGEVQLLESGGGLVQPGGSLTLSCAASGRSFSN |
| | | YIMGWFRQAPGKEREFVAGLGWSPGNTYYADSVKGRFTISRDNSKN |
| | | TLYLQMNSLRAEDTAVYYCAARRGDVIYTTPWNYVYWGLGTQVTVS |
| | | STSDYKDDDDKTTTPAPRPPTPAPTIASQPLSLRPEACRPAAGGAVHT |
| | | RGLDFACDIYIWAPLAGTCGVLLLSLVITLYKRGRKKLLYIFKQPFMRPV |
| | | QTTQEEDGCSCRFPEEEEGGCELRVKFSRSADAPAYQQGQNQLYNEL |
| | | NLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 47. | C2031 | MALPVTALLLPLALLLHAARPEVQLVESGGGLVQPGNSLRLSCAASGF |
| | | TFSKFGMSWVRQANPWVPPREGLEWVSSISGSGRDTLYADSVKGRF |
| | | TISRDNAKTTLYLQMNSLRPEDTAVYYCTIGGSLSVSSQGTLVTVSSGG |
| | | GGLSGRSDNHGGGGTEVQLLESGGGLVQPGGSLTLSCAASGRSFSNY |
| | | IMGWFRQAPGKEREFVAGLGWSPGNTYYADSVKGRFTISRDNSKNT |
| | | LYLQMNSLRAEDTAVYYCAARRGDVIYTTPWNYVYWGLGTQVTVSS |
| | | TSDYKDDDDKTTTPAPRPPTPAPTIASQPLSLRPEACRPAAGGAVHTR |
| | | GLDFACDIYIWAPLAGTCGVLLLSLVITLYKRGRKKLLYIFKQPFMRPVQ |
| | | TTQEEDGCSCRFPEEEEGGCELRVKFSRSADAPAYQQGQNQLYNELN |
| | | LGRREEYDVLDKRRGRDPEMGGKPRRKNPQEGLYNELQKDKMAEAY |
| | | SEIGMKGERRRGKGHDGLYQGLSTATKDTYDALHMQALPPR |
| 48. | C1827 | MALPVTALLLPLALLLHAARPEVQLLESGGGLVQPGGSLTLSCAASGR |
| | | TDSWYVMGWFRQAPGKDREFVAGVSWSYGNTYYADSVKGRFTISR |
| | | DNSKNTLYLQMNSLRAEDTAVYYCAARVSREVIPTRWDLYNYWGLG |
| | | TQVTVSSTSDYKDDDDKTTTPAPRPPTPAPTIASQPLSLRPEACRPAA |
| | | GGAVHTRGLDFACDIYIWAPLAGTCGVLLLSLVITLYKRGRKKLLYIFKQ |
| | | PFMRPVQTTQEEDGCSCRFPEEEEGGCELRVKFSRSADAPAYQQGQ |
| | | NQLYNELNLGRREEYDVLDKRRGRDPEMGGKPRRKNPQEGLYNELQ |
| | | KDKMAEAYSEIGMKGERRRGKGHDGLYQGLSTATKDTYDALHMQA |
| | | LPPR |
| 49. | C1829 | MALPVTALLLPLALLLHAARPEVQLVESGGGLVQPGNSLRLSCAASGF |
| | | TFSKFGMSWVRQAPGKGLEWVSSISGSGRDTLYADSVKGRFTISRDN |
| | | AKTTLYLQMNSLRPEDTAVYYCTIGGSLSVSSQGTLVTVSSGSGGSGG |
| | | GGSGGGGGSGEVQLLESGGGLVQPGGSLTLSCAASGRTDSWYVMG |
| | | WFRQAPGKDREFVAGVSWSYGNTYYADSVKGRFTISRDNSKNTLYL |
| | | QMNSLRAEDTAVYYCAARVSREVIPTRWDLYNYWGLGTQVTVSSTS |
| | | DYKDDDDKTTTPAPRPPTPAPTIASQPLSLRPEACRPAAGGAVHTRGL |
| | | DFACDIYIWAPLAGTCGVLLLSLVITLYKRGRKKLLYIFKQPFMRPVQTT |
| | | QEEDGCSCRFPEEEEGGCELRVKFSRSADAPAYQQGQNQLYNELNLG |
| | | RREEYDVLDKRRGRDPEMGGKPRRKNPQEGLYNELQKDKMAEAYSE |
| | | IGMKGERRRGKGHDGLYQGLSTATKDTYDALHMQALPPR |
| 50. | C1952 | MALPVTALLLPLALLLHAARPEVQLVESGGGLVQPGNSLRLSCAASGF |
| | | TFSKFGMSWVRQAHEPLTCRGLEWVSSISGSGRDTLYADSVKGRFTIS |
| | | RDNAKTTLYLQMNSLRPEDTAVYYCTIGGSLSVSSQGTLVTVSSGSGG |
| | | SGGGGSGGGGGSGEVQLLESGGGLVQPGGSLTLSCAASGRTDSWYV |
| | | MGWFRQAPGKDREFVAGVSWSYGNTYYADSVKGRFTISRDNSKNTL |
| | | YLQMNSLRAEDTAVYYCAARVSREVIPTRWDLYNYWGLGTQVTVSS |
| | | TSDYKDDDDKTTTPAPRPPTPAPTIASQPLSLRPEACRPAAGGAVHTR |
| | | GLDFACDIYIWAPLAGTCGVLLLSLVITLYKRGRKKLLYIFKQPFMRPVQ |
| | | TTQEEDGCSCRFPEEEEGGCELRVKFSRSADAPAYQQGQNQLYNELN |
| | | LGRREEYDVLDKRRGRDPEMGGKPRRKNPQEGLYNELQKDKMAEAY |
| | | SEIGMKGERRRGKGHDGLYQGLSTATKDTYDALHMQALPPR |
| 51. | C1954 | MALPVTALLLPLALLLHAARPEVQLVESGGGLVQPGNSLRLSCAASGF |
| | | TFSKFGMSWVRQASHPMSCRGLEWVSSISGSGRDTLYADSVKGRFTI |
| | | SRDNAKTTLYLQMNSLRPEDTAVYYCTIGGSLSVSSQGTLVTVSSGSG |
| | | GSGGGGSGGGGGSGEVQLLESGGGLVQPGGSLTLSCAASGRTDSWY |
| | | VMGWFRQAPGKDREFVAGVSWSYGNTYYADSVKGRFTISRDNSKN |
| | | TLYLQMNSLRAEDTAVYYCAARVSREVIPTRWDLYNYWGLGTQVTVS |
| | | STSDYKDDDDKTTTPAPRPPTPAPTIASQPLSLRPEACRPAAGGAVHT |
| | | RGLDFACDIYIWAPLAGTCGVLLLSLVITLYKRGRKKLLYIFKQPFMRPV |
| | | QTTQEEDGCSCRFPEEEEGGCELRVKFSRSADAPAYQQGQNQLYNEL |
| | | NLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 52. | C2033 | MALPVTALLLPLALLLHAARPEVQLVESGGGLVQPGNSLRLSCAASGF |
| | | TFSKFGMSWVRQAHEPLTCRGLEWVSSISGSGRDTLYADSVKGRFTIS |
| | | RDNAKTTLYLQMNSLRPEDTAVYYCTIGGSLSVSSQGTLVTVSSGGGG |
| | | LSGRSDNHGGGGTEVQLLESGGGLVQPGGSLTLSCAASGRTDSWYV |
| | | MGWFRQAPGKDREFVAGVSWSYGNTYYADSVKGRFTISRDNSKNTL |
| | | YLQMNSLRAEDTAVYYCAARVSREVIPTRWDLYNYWGLGTQVTVSS |
| | | TSDYKDDDDKTTTPAPRPPTPAPTIASQPLSLRPEACRPAAGGAVHTR |
| | | GLDFACDIYIWAPLAGTCGVLLLSLVITLYKRGRKKLLYIFKQPFMRPVQ |
| | | TTQEEDGCSCRFPEEEEGGCELRVKFSRSADAPAYQQGQNQLYNELN |
| | | LGRREEYDVLDKRRGRDPEMGGKPRRKNPQEGLYNELQKDKMAEAY |
| | | SEIGMKGERRRGKGHDGLYQGLSTATKDTYDALHMQALPPR |
| 53. | C1953 | MALPVTALLLPLALLLHAARPEVQLVESGGGLVQPGNSLRLSCAASGF |
| | | TFSKFGMSWVRQASHPMSCRGLEWVSSISGSGRDTLYADSVKGRFTI |
| | | SRDNAKTTLYLQMNSLRPEDTAVYYCTIGGSLSVSSQGTLVTVSSGSG |
| | | GPLGLQARVVGGSGEVQLLESGGGLVQPGGSLTLSCAASGRTDSWY |
| | | VMGWFRQAPGKDREFVAGVSWSYGNTYYADSVKGRFTISRDNSKN |
| | | TLYLQMNSLRAEDTAVYYCAARVSREVIPTRWDLYNYWGLGTQVTVS |
| | | STSDYKDDDDKTTTPAPRPPTPAPTIASQPLSLRPEACRPAAGGAVHT |
| | | RGLDFACDIYIWAPLAGTCGVLLLSLVITLYKRGRKKLLYIFKQPFMRPV |
| | | QTTQEEDGCSCRFPEEEEGGCELRVKFSRSADAPAYQQGQNQLYNEL |
| | | NLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 54. | C2035 | MALPVTALLLPLALLLHAARPEVQLVESGGGLVQPGNSLRLSCAASGF |
| | | TFSKFGMSWVRQASHPMSCRGLEWVSSISGSGRDTLYADSVKGRFTI |
| | | SRDNAKTTLYLQMNSLRPEDTAVYYCTIGGSLSVSSQGTLVTVSSGGG |
| | | GLSGRSDNHGGGGTEVQLLESGGGLVQPGGSLTLSCAASGRTDSWY |
| | | VMGWFRQAPGKDREFVAGVSWSYGNTYYADSVKGRFTISRDNSKN |
| | | TLYLQMNSLRAEDTAVYYCAARVSREVIPTRWDLYNYWGLGTQVTVS |
| | | STSDYKDDDDKTTTPAPRPPTPAPTIASQPLSLRPEACRPAAGGAVHT |
| | | RGLDFACDIYIWAPLAGTCGVLLLSLVITLYKRGRKKLLYIFKQPFMRPV |
| | | QTTQEEDGCSCRFPEEEEGGCELRVKFSRSADAPAYQQGQNQLYNEL |
| | | NLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 55. | CD8a signal peptide | MALPVTALLLPLALLLHAARP |
| 56. | 10G sdAb | EVQLVESGGGLVQPGNSLRLSCAASGFTFSKFGMSWVRQAPGKGLE |
| | | WVSSISGSGRDTLYADSVKGRFTISRDNAKTTLYLQMNSLRPEDTAVY |
| | | YCTIGGSLSVSSQGTLVTVSS |
| 57. | 10G sdAb EH4 Mask 1 | EVQLVESGGGLVQPGNSLRLSCAASGFTFSKFGMSWVRQANPWVP |
| | | PREGLEWVSSISGSGRDTLYADSVKGRFTISRDNAKTTLYLQMNSLRP |
| | | EDTAVYYCTIGGSLSVSSQGTLVTVSS |
| 58. | 10G sdAb EH104 | EVQLVESGGGLVQPGNSLRLSCAASGFTFSKFGMSWVRQAHEPLTC |
| | Mask 1 | RGLEWVSSISGSGRDTLYADSVKGRFTISRDNAKTTLYLQMNSLRPED |
| | | TAVYYCTIGGSLSVSSQGTLVTVSS |
| 59. | 10G sdAb EH104 | EVQLVESGGGLVQPGNSLRLSCAASGFTFSKFGMSWVRQASHPMSC |
| | Mask 2 | RGLEWVSSISGSGRDTLYADSVKGRFTISRDNAKTTLYLQMNSLRPED |
| | | TAVYYCTIGGSLSVSSQGTLVTVSS |
| 60. | EH4 sdAb | EVQLLESGGGLVQPGGSLTLSCAASGRSFSNYIMGWFRQAPGKEREF |
| | | VAGLGWSPGNTYYADSVKGRFTISRDNSKNTLYLQMNSLRAEDTAVY |
| | | YCAARRGDVIYTTPWNYVYWGLGTQVTVSS |
| 61. | EH104 sdAb | EVQLLESGGGLVQPGGSLTLSCAASGRTDSWYVMGWFRQAPGKDR |
| | | EFVAGVSWSYGNTYYADSVKGRFTISRDNSKNTLYLQMNSLRAEDTA |
| | | VYYCAARVSREVIPTRWDLYNYWGLGTQVTVSS |
| 62. | CD8a hinge/TM | TTTPAPRPPTPAPTIASQPLSLRPEACRPAAGGAVHTRGLDFACDIYIW |
| | | APLAGTCGVLLLSLVITLY |
| 63. | 4-1BB intracellular | KRGRKKLLYIFKQPFMRPVQTTQEEDGCSCRFPEEEEGGCEL |
| | domain | |
| 64. | CD3z intracellular | RVKFSRSADAPAYQQGQNQLYNELNLGRREEYDVLDKRRGRDPEMG |
| | domain | GKPRRKNPQEGLYNELQKDKMAEAYSEIGMKGERRRGKGHDGLYQ |
| | | GLSTATKDTYDALHMQALPPR |
| 65. | non-cleavable linker | GSGGSGGGGSGGGGGSG |
| 66. | protease cleavable | GGGGLSGRSDNHGGGGT |
| | linker 1 | |
| 67. | protease cleavable | GSGGPLGLQARVVGGSG |
| | linker 2 | |
| 68. | EH4 Mask 1 | ANPWVPPREG |
| 69. | EH104 Mask 1 | AHEPLTCRG |
| 70. | EH104 Mask 2 | ASHPMSCRG |
| 71. | C2446 | MALPVTALLLPLALLLHAARPEVQLVESGGGLVQPGNSLRLSCAASGF |
| | | TFSKFGMSWVRQANPWVPPREGLEWVSSISGSGRDTLYADSVKGRF |
| | | TISRDNAKTTLYLQMNSLRPEDTAVYYCTIGGSLSVSSQGTLVTVSSGG |
| | | GGPPASSGRAGGGGGGTEVQLLESGGGLVQPGGSLTLSCAASGRSFS |
| | | NYIMGWFRQAPGKEREFVAGLGWSPGNTYYADSVKGRFTISRDNSK |
| | | NTLYLQMNSLRAEDTAVYYCAARRGDVIYTTPWNYVYWGLGTQVTV |
| | | SSTSDYKDDDDKTTTPAPRPPTPAPTIASQPLSLRPEACRPAAGGAVH |
| | | TRGLDFACDIYIWAPLAGTCGVLLLSLVITLYKRGRKKLLYIFKQPFMRP |
| | | VQTTQEEDGCSCRFPEEEEGGCELRVKFSRSADAPAYQQGQNQLYN |
| | | ELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 72. | C2448 | MALPVTALLLPLALLLHAARPEVQLLESGGGLVQPGGSLTLSCAASGR |
| | | SFSNYIMGWFRQAPGKEREFVAGLGWSPGNTYYADSVKGRFTISRD |
| | | NSKNTLYLQMNSLRAEDTAVYYCAARRGDVIYTTPWNYVYWGLGTQ |
| | | VTVSSTSDYKDDDDKTTTPAPRPPTPAPTIASQPLSLRPEACRPAAGG |
| | | AVHTRGLDFASDIYIWAPLAGTCGVLLLSLVITLYKRGRKKLLYIFKQPF |
| | | MRPVQTTQEEDGCSCRFPEEEEGGCELRVKFSRSADAPAYQQGQNQ |
| | | LYNELNLGRREEYDVLDKRRGRDPEMGGKPRRKNPQEGLYNELQKD |
| | | KMAEAYSEIGMKGERRRGKGHDGLYQGLSTATKDTYDALHMQALPP |
| | | R |
| 73. | C2510 | MALPVTALLLPLALLLHAARPEVQLVESGGGLVQPGNSLRLSCAASGF |
| | | TFSKFGMSWVRQANPWVPPREGLEWVSSISGSGRDTLYADSVKGRF |
| | | TISRDNAKTTLYLQMNSLRPEDTAVYYCTIGGSLSVSSQGTLVTVSSGG |
| | | GGLSGRSDNHGGGGTEVQLLESGGGLVQPGGSLTLSCAASGRSFSNY |
| | | IMGWFRQAPGKEREFVAGLGWSPGNTYYADSVKGRFTISRDNSKNT |
| | | LYLQMNSLRAEDTAVYYCAARRGDVIYTTPWNYVYWGLGTQVTVSS |
| | | TSDYKDDDDKTTTPAPRPPTPAPTIASQPLSLRPEACRPAAGGAVHTR |
| | | GLDFASDIYIWAPLAGTCGVLLLSLVITLYKRGRKKLLYIFKQPFMRPVQ |
| | | TTQEEDGCSCRFPEEEEGGCELRVKFSRSADAPAYQQGQNQLYNELN |
| | | LGRREEYDVLDKRRGRDPEMGGKPRRKNPQEGLYNELQKDKMAEAY |
| | | SEIGMKGERRRGKGHDGLYQGLSTATKDTYDALHMQALPPR |
| 74. | C2513 | MALPVTALLLPLALLLHAARPEVQLVESGGGLVQPGNSLRLSCAASGF |
| | | TFSKFGMSWVRQANPWVPPREGLEWVSSISGSGRDTLYADSVKGRF |
| | | TISRDNAKTTLYLQMNSLRPEDTAVYYCTIGGSLSVSSQGTLVTVSSGG |
| | | GGPPASSGRAGGGGGGTEVQLLESGGGLVQPGGSLTLSCAASGRSFS |
| | | NYIMGWFRQAPGKEREFVAGLGWSPGNTYYADSVKGRFTISRDNSK |
| | | NTLYLQMNSLRAEDTAVYYCAARRGDVIYTTPWNYVYWGLGTQVTV |
| | | SSTSDYKDDDDKTTTPAPRPPTPAPTIASQPLSLRPEACRPAAGGAVH |
| | | TRGLDFASDIYIWAPLAGTCGVLLLSLVITLYKRGRKKLLYIFKQPFMRP |
| | | VQTTQEEDGCSCRFPEEEEGGCELRVKFSRSADAPAYQQGQNQLYN |
| | | ELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 75. | C2514 | MALPVTALLLPLALLLHAARPEVQLVESGGGLVQPGNSLRLSCAASGF |
| | | TFSKFGMSWVRQANPWVPPREGLEWVSSISGSGRDTLYADSVKGRF |
| | | TISRDNAKTTLYLQMNSLRPEDTAVYYCTIGGSLSVSSQGTLVTVSSGS |
| | | GGSGGGGSGGGGGSGEVQLLESGGGLVQPGGSLTLSCAASGRSFSN |
| | | YIMGWFRQAPGKEREFVAGLGWSPGNTYYADSVKGRFTISRDNSKN |
| | | TLYLQMNSLRAEDTAVYYCAARRGDVIYTTPWNYVYWGLGTQVTVS |
| | | STSDYKDDDDKTTTPAPRPPTPAPTIASQPLSLRPEACRPAAGGAVHT |
| | | RGLDFASDIYIWAPLAGTCGVLLLSLVITLYKRGRKKLLYIFKQPFMRPV |
| | | QTTQEEDGCSCRFPEEEEGGCELRVKFSRSADAPAYQQGQNQLYNEL |
| | | NLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 76. | C2780 | MALPVTALLLPLALLLHAARPQVQLVESGGALVQPGGSLRLSCAASGF |
| | | PVNRYSMRWYRQAPGKEREWVAGMSSAGDRSSYEDSVKGRFTISR |
| | | DDARNTVYLQMNSLKPEDTAVYYCNVNVGFEYWGQGTQVTVSSTS |
| | | DYKDDDDKTTTPAPRPPTPAPTIASQPLSLRPEACRPAAGGAVHTRGL |
| | | DFACDIYIWAPLAGTCGVLLLSLVITLYKRGRKKLLYIFKQPFMRPVQTT |
| | | QEEDGCSCRFPEEEEGGCELRVKFSRSADAPAYQQGQNQLYNELNLG |
| | | RREEYDVLDKRRGRDPEMGGKPRRKNPQEGLYNELQKDKMAEAYSE |
| | | IGMKGERRRGKGHDGLYQGLSTATKDTYDALHMQALPPR |
| 77. | C2483 | MALPVTALLLPLALLLHAARPEVQLLESGGGLVQPGGSLTLSCAASGFI |
| | | FRAASMAWYRQSPGNERELVASISSGAFTNYADSVKARFTISRDNSK |
| | | NTLYLQMNSLRAEDTAVYYCGATFLRSDGHHTINGQGTLVTVSSTSD |
| | | YKDDDDKTTTPAPRPPTPAPTIASQPLSLRPEACRPAAGGAVHTRGLD |
| | | FACDIYIWAPLAGTCGVLLLSLVITLYKRGRKKLLYIFKQPFMRPVQTTQ |
| | | EEDGCSCRFPEEEEGGCELRVKFSRSADAPAYQQGQNQLYNELNLGR |
| | | REEYDVLDKRRGRDPEMGGKPRRKNPQEGLYNELQKDKMAEAYSEI |
| | | GMKGERRRGKGHDGLYQGLSTATKDTYDALHMQALPPR |
| 78. | C2790 | MALPVTALLLPLALLLHAARPEVQLVESGGGLVQPGNSLRLSCAASGF |
| | | TFSKFGMSWVRQAPGKGLEWVSSISGSGRDTLYADSVKGRFTISRDN |
| | | AKTTLYLQMNSLRPEDTAVYYCTIGGSLSVSSQGTLVTVSSGSGGSGG |
| | | GGSGGGGGSGEVQLLESGGGLVQPGGSLTLSCAASGFIFRAASMAW |
| | | YRQSPGNERELVASISSGAFTNYADSVKARFTISRDNSKNTLYLQMNS |
| | | LRAEDTAVYYCGATFLRSDGHHTINGQGTLVTVSSTSDYKDDDDKTTT |
| | | PAPRPPTPAPTIASQPLSLRPEACRPAAGGAVHTRGLDFACDIYIWAP |
| | | LAGTCGVLLLSLVITLYKRGRKKLLYIFKQPFMRPVQTTQEEDGCSCRF |
| | | PEEEEGGCELRVKFSRSADAPAYQQGQNQLYNELNLGRREEYDVLDK |
| | | RRGRDPEMGGKPRRKNPQEGLYNELQKDKMAEAYSEIGMKGERRR |
| | | GKGHDGLYQGLSTATKDTYDALHMQALPPR |
| 79. | C3269 | MALPVTALLLPLALLLHAARPEVQLVESGGGLVQPGNSLRLSCAASGF |
| | | TFSKFGMSWVRQRQSTFMAMDMPILEWVSSISGSGRDTLYADSVK |
| | | GRFTISRDNAKTTLYLQMNSLRPEDTAVYYCTIGGSLSVSSQGTLVTVS |
| | | SGSGGSGGGGSGGGGGSGEVQLLESGGGLVQPGGSLTLSCAASGFIF |
| | | RAASMAWYRQSPGNERELVASISSGAFTNYADSVKARFTISRDNSKN |
| | | TLYLQMNSLRAEDTAVYYCGATFLRSDGHHTINGQGTLVTVSSTSDYK |
| | | DDDDKTTTPAPRPPTPAPTIASQPLSLRPEACRPAAGGAVHTRGLDFA |
| | | CDIYIWAPLAGTCGVLLLSLVITLYKRGRKKLLYIFKQPFMRPVQTTQEE |
| | | DGCSCRFPEEEEGGCELRVKFSRSADAPAYQQGQNQLYNELNLGRRE |
| | | EYDVLDKRRGRDPEMGGKPRRKNPQEGLYNELQKDKMAEAYSEIG |
| | | MKGERRRGKGHDGLYQGLSTATKDTYDALHMQALPPR |
| 80. | C3272 | MALPVTALLLPLALLLHAARPEVQLVESGGGLVQPGNSLRLSCAASGF |
| | | TFSKFGMSWVRQQAYWWGNSRDLEWVSSISGSGRDTLYADSVKGR |
| | | FTISRDNAKTTLYLQMNSLRPEDTAVYYCTIGGSLSVSSQGTLVTVSSG |
| | | SGGSGGGGSGGGGGSGEVQLLESGGGLVQPGGSLTLSCAASGFIFRA |
| | | ASMAWYRQSPGNERELVASISSGAFTNYADSVKARFTISRDNSKNTLY |
| | | LQMNSLRAEDTAVYYCGATFLRSDGHHTINGQGTLVTVSSTSDYKDD |
| | | DDKTTTPAPRPPTPAPTIASQPLSLRPEACRPAAGGAVHTRGLDFACD |
| | | IYIWAPLAGTCGVLLLSLVITLYKRGRKKLLYIFKQPFMRPVQTTQEEDG |
| | | CSCRFPEEEEGGCELRVKFSRSADAPAYQQGQNQLYNELNLGRREEY |
| | | DVLDKRRGRDPEMGGKPRRKNPQEGLYNELQKDKMAEAYSEIGMK |
| | | GERRRGKGHDGLYQGLSTATKDTYDALHMQALPPR |
| 81. | C3328 | MALPVTALLLPLALLLHAARPEVQLVESGGGLVQPGNSLRLSCAASGF |
| | | TFSKFGMSWVRQQAYWWGNSRDLEWVSSISGSGRDTLYADSVKGR |
| | | FTISRDNAKTTLYLQMNSLRPEDTAVYYCTIGGSLSVSSQGTLVTVSSG |
| | | GGGPQGSTGRAAGGGGGTEVQLLESGGGLVQPGGSLTLSCAASGFIF |
| | | RAASMAWYRQSPGNERELVASISSGAFTNYADSVKARFTISRDNSKN |
| | | TLYLQMNSLRAEDTAVYYCGATFLRSDGHHTINGQGTLVTVSSTSDYK |
| | | DDDDKTTTPAPRPPTPAPTIASQPLSLRPEACRPAAGGAVHTRGLDFA |
| | | CDIYIWAPLAGTCGVLLLSLVITLYKRGRKKLLYIFKQPFMRPVQTTQEE |
| | | DGCSCRFPEEEEGGCELRVKFSRSADAPAYQQGQNQLYNELNLGRRE |
| | | EYDVLDKRRGRDPEMGGKPRRKNPQEGLYNELQKDKMAEAYSEIG |
| | | MKGERRRGKGHDGLYQGLSTATKDTYDALHMQALPPR |
| 82. | C3329 | MALPVTALLLPLALLLHAARPEVQLVESGGGLVQPGNSLRLSCAASGF |
| | | TFSKFGMSWVRQRQSTFMAMDMPILEWVSSISGSGRDTLYADSVK |
| | | GRFTISRDNAKTTLYLQMNSLRPEDTAVYYCTIGGSLSVSSQGTLVTVS |
| | | SGGGGPQGSTGRAAGGGGGTEVQLLESGGGLVQPGGSLTLSCAASG |
| | | FIFRAASMAWYRQSPGNERELVASISSGAFTNYADSVKARFTISRDNS |
| | | KNTLYLQMNSLRAEDTAVYYCGATFLRSDGHHTINGQGTLVTVSSTS |
| | | DYKDDDDKTTTPAPRPPTPAPTIASQPLSLRPEACRPAAGGAVHTRGL |
| | | DFACDIYIWAPLAGTCGVLLLSLVITLYKRGRKKLLYIFKQPFMRPVQTT |
| | | QEEDGCSCRFPEEEEGGCELRVKFSRSADAPAYQQGQNQLYNELNLG |
| | | RREEYDVLDKRRGRDPEMGGKPRRKNPQEGLYNELQKDKMAEAYSE |
| | | IGMKGERRRGKGHDGLYQGLSTATKDTYDALHMQALPPR |
| 83. | dimerization | TTTPAPRPPTPAPTIASQPLSLRPEACRPAAGGAVHTRGLDFASDIYIW |
| | deficient CD8a | APLAGTCGVLLLSLVITLY |
| | hinge/TM | |
| 84. | protease cleavable | GGGGPPASSGRAGGGGGGT |
| | linker 3 | |
| 85. | 10G sdAb H90 Mask 1 | EVQLVESGGGLVQPGNSLRLSCAASGFTFSKFGMSWVRQRQSTFMA |
| | | MDMPILEWVSSISGSGRDTLYADSVKGRFTISRDNAKTTLYLQMNSL |
| | | RPEDTAVYYCTIGGSLSVSSQGTLVTVSS |
| 86. | 10G sdAb H90 Mask 2 | EVQLVESGGGLVQPGNSLRLSCAASGFTFSKFGMSWVRQQAYWWG |
| | | NSRDLEWVSSISGSGRDTLYADSVKGRFTISRDNAKTTLYLQMNSLRP |
| | | EDTAVYYCTIGGSLSVSSQGTLVTVSS |
| 87. | H90 sdAb | EVQLLESGGGLVQPGGSLTLSCAASGFIFRAASMAWYRQSPGNEREL |
| | | VASISSGAFTNYADSVKARFTISRDNSKNTLYLQMNSLRAEDTAVYYC |
| | | GATFLRSDGHHTINGQGTLVTVSS |
| 88. | GFP sdAb | QVQLVESGGALVQPGGSLRLSCAASGFPVNRYSMRWYRQAPGKERE |
| | | WVAGMSSAGDRSSYEDSVKGRFTISRDDARNTVYLQMNSLKPEDTA |
| | | VYYCNVNVGFEYWGQGTQVTVSS |
| 89. | H90 Mask 1 | RQSTFMAMDMPI |
| 90. | H90 Mask 2 | QAYWWGNSRD |
| 91. | metalloproteinase | Gly-Pro-Leu-Gly-Ile-Ala-Gly-Gln |
| | cleavage site | |
| 92. | metalloproteinase | Ala-Val-Arg-Trp-Leu-Leu-Thr-Ala |
| | cleavage site | |
| 93. | furin cleavage site | Arg-Arg-Arg-Arg-Arg-Arg |
| 94. | Exemplary | IYIWAPLAGTCGVLLLSLVITLYC(SEQ ID NO: 94) |
| | transmembrane (TM) | |
| | sequence | |
| 95. | CD8 beta derived TM | GLLVAGVLVLLVSLGVAIHLCC |
| 96. | CD4 derived TM | ALIVLGGVAGLLLFIGLGIFFCVRC |
| 97. | CD3 zeta derived TM | LCYLLDGILFIYGVILTALFLRV |
| 98. | CD28 derived TM | WVLVVVGGVLACYSLLVTVAFIIFWV |
| 99. | CD134 (OX40) | AAILGLGLVLGLLGPLAILLALYLL |
| | derived TM: | |
| 100. | CD7 derived TM | ALPAALAVISFLLGLGLGVACVLA |
| 101. | Hinge region | CPEPKSCDTPPPCPR |
| 102. | Hinge region | ELKTPLGDTTHT |
| 103. | Hinge region | KCCVDCP |
| 104. | Hinge region | KYGPPCP |
| 105. | Human IgG1 hinge | EPKSCDKTHTCPPCP |
| | region | |
| 106. | Human IgG2 hinge | ERKCCVECPPCP |
| | region | |
| 107. | Human IgG3 hinge | ELKTPLGDTTHTCPRCP |
| | region | |
| 108. | Human IgG4 hinge | SPNMVPHAHHAQ |
| | region | |
| 109. | His229 of Human | EPKSCDKTYTCPPCP |
| | IgG1 hinge region | |
| | substituted with Tyr | |
| 110. | CD8 hinge region | TTTPAPRPPTPAPTIASQPLSLRPEACRPAAGGAVHTRGLDFACD |
| 111. | Hinge region | KSCDKTHTCP |
| 112. | EPL90 Anti-EPCAM | QVQLQESGGGLVQAGGSLRLSCAASGFIFRAASMAWYRQSPGNERE |
| | sequence | LVASISSGAFTNYADSVKARFTISRDNAKNTVYLQMNSLKPEDTAVYF |
| | | CGATFLRSDGHHTINGQGTQVTVSS |
| 113. | EPL118 Anti-EPCAM | QVQLQESGGGLVQAGGSLRLSCAASGFIFRAASMGWFRQSPGNERE |
| | sequence | LVATVSSGDFTNYADSVKGRFTISRDNAKNTVYLQMNSLKPEDTAVYF |
| | | CGATFVRSDGHHTIYGQGTQVTVSS |
| 114. | EPL138 Anti-EPCAM | QVQLQESGGGLVQAGGSLRLSCAASGFIFRAASMDWYRQFPGNERE |
| | sequence | SIATISSGGFTNYADSVKGRFTISRDNAKNTVYLQMNSLKPEDTAVYFC |
| | | GATFLRSDGHHTINGQGTQVTVSS |
| 115. | EPL145 Anti-EPCAM | QVQLQESGGGLVQAGGSLRLSCAASGFIFRAASMGWFRQSPGNERE |
| | sequence | LVATVSSGGFTNYADSVKGRFTISRDNAKNTVYLQMNSLKPEDTAVY |
| | | FCGATFVRSDGHHTIYGQGTQVTVSS |
| 116. | EPL164 Anti-EPCAM | QVQLQESGGGLVQAGGSLRLSCAASGFIFRAASMDWYRQSPGTQPE |
| | sequence | LVATISSTGFTNYANSVKGRFTISRDNAKNTVYLQMNSLKPEDTAVYF |
| | | CGATFLRSDGQHSIYGQGTQVTVSS |
| 117. | EPL31 Anti-EPCAM | QVQLQESGGGLVHTGGSLRLSCAASGDTFLRYAMGWFRQAPGKERE |
| | sequence | FVAAITWNGGNTDYAGSLKGRFTISRDNTKNTVYLQMNSLKPEDTAV |
| | | YYCAADLTFGLASSHYQYDYWGQGTQVTVSS |
| 118. | EPL55 Anti-EPCAM | QVQLQESGGGLVQAGGSLRLSCAASGDTFLRYAMGWFRQAPGKER |
| | sequence | EFVAAITWNGGNTDYAGSLKGRFTISRDNTKNTVYLQMNSLKPEDTA |
| | | VYYCAADLTFGLASSHYQYDYWGQGTQVTVSS |
| 119. | EPL57 Anti-EPCAM | QVQLQESGGGLVHTGGSLRLSCAFSGDTFLRYAMGWFRQAPGKERE |
| | sequence | FVAAITWNGGNTDYADSLKGRFTISRDNTKNTVYLQMNSLRPEDTAV |
| | | YYCAADLTFGLASSHYQYDYWGQGTQVTVSS |
| 120. | EPL136 Anti-EPCAM | QVQLQESGGGLVQPGGSLRLSCAASGDTFLRYAMGWFRQAPGKER |
| | sequence | EFVAAITWNGGNTDYAGSLKGRFTISRDNTKNTVYLQMNSLKPEDTA |
| | | VYYCAADLTFGLASSHYQYDYWGQGTQVTVSS |
| 121. | EPL15 Anti-EPCAM | QVQLQESGGGSVLAGGSLRLSCAASGFTFSSYYMSWVRQAPGKGLE |
| | sequence | WVSGIHYTGDWTNYADSVKGRFTISRDNAKNELYLEMNNLKPEDTA |
| | | VYYCARGSDKGQGTQVTVSS |
| 122. | EPL34 Anti-EPCAM | QVQLQESGGGSVQAGGSLRLSCAASGFTFSSYYMSWVRQAPGKGLE |
| | sequence | WVSGIHYTGDWTNYADSVKGRFTISRDNAKNELYLEMNNLKPEDTA |
| | | VYYCARGSDKGQGTQVTVSS |
| 123. | EPL86 Anti-EPCAM | QVQLQESGGGLVQPGGSLRLSCAASGFTFSDWAMSWVRQAPGKGL |
| | sequence | EWVSGIHYGDHTTHYADFVKGRFTISRDDAKNTLYLQMNSLKPEDTA |
| | | VYYCARGSTKGQGTQVTVSS |
| 124. | EPL153 Anti-EPCAM | QVQLQESGGGLVQPGGSLRLSCAASGFTFSDWAMSWVRQAPGKGL |
| | sequence | EWVSSIHYGDHTTHYADFVKGRFTISRDDAKNTLYLQMNSLKPEDTA |
| | | VYYCEKGTTRGQGTQVTVSS |
| 125. | EPL20 Anti-EPCAM | QVQLQESGGGLVQAGGSLKLSCAASGNVFRAATMAWYRQAPEKQR |
| | sequence | EMVATIASGGTTNYADFVKGRFTISRDNAKNTVYLQMNTLKPEDTAV |
| | | YYCNAGYLTSLGPKNYWGQGTQVTVSS |
| 126. | EPL70 Anti-EPCAM | QVQLQESGGGLVQPGGSLRLSCAASGNVFRAATMAWYRQAPEKXR |
| | sequence | EMVATIASGGTTNYADFVKGRFTISRDNAKNTVYLQMNTLKPEDTAV |
| | | YYCNAGYLTSLGPKNYWGQGTQVTVSS |
| 127. | EPL125 Anti-EPCAM | QVQLQESGGGLVQAGGSLRLSCAASGNVFRAATMAWYRQVPEKQR |
| | sequence | EMVATIASGGTTNYADFVKGRFTISRDNAKNTVYLQMNTLKPEDTAV |
| | | YYCNALYLTSLGPKSYWGQGTQVTVSS |
| 128. | EPL13 Anti-EPCAM | QVQLQESGGGLVQPGGSLRLSCAASGFAFGNHWMYWYRQAPGRG |
| | sequence | RELVASISSGGSTNYVDSVKGRFTISRDNARNTVYLQMYSLKPEDTAV |
| | | YYCGTSDNWGQGTQVTVSS |
| 129. | EPL129 Anti-EPCAM | QVQLQESGGGLVQAGGSLRLSCAASGFAFGNHWMYWYRQAPGRG |
| | sequence | RELVASISSGGSTNYVDSVKGRFTISRDNARNTVYLQMYSLRPEDTAV |
| | | YYCGTSDNWGQGTQVTVSS |
| 130. | EPL159 Anti-EPCAM | QVQLQESGGGLVQAGGSLRLSCAASGFAFGNHWMYWYRQAPGRG |
| | sequence | RELVASISSGGSTNYVDSVKGRFTISRDNARNTVYLQMYSLKPEDTAV |
| | | YYCGTSDNWGQGTQVTVSS |
| 131. | EPL120 Anti-EPCAM | QVQLQESGGGLVQAGGSLRLSCAASGFIFRAASISWYRQSPGNEREL |
| | sequence | VATINSGGFTNYADSVLGRFTISRDNAKNTGYLQMNSLKPEDTAVYFC |
| | | AATFLRSDGQPPIWGQGTQVTVSS |
| 132. | EPL126 Anti-EPCAM | QVQLQESGGGLVQAGGSLRLSCAASGFIFRAASMGWYRQSPGNERE |
| | sequence | LVATINSGGFTNYADSVKGRFTISRDNAKNTGYLQMNSLKPEDTAVYF |
| | | CAATFLRSDGQPPIWGQGTQVTVSS |
| 133. | EPL60 Anti-EPCAM | QVQLQESGGGLVQAGGSLRLSCAASEYILSMYRMAWYRQAPGKVRE |
| | sequence | LVADMSSGGTTNYADFVKGRFTISRDNDRNTVYLQMNRLQPEDTAA |
| | | YYCNVAGRTGPPSYDAFNNWGQGTQVTVSS |
| 134. | EPL156 Anti-EPCAM | QVQLQESGGGLVQPGASLRVSCAASEYILSMYRMAWYRQAPGKVRE |
| | sequence | LVADMSSGGTTNYADFVKGRFTISRDNDRNTVYLQMNRLQPEDTAA |
| | | YYCNVAGRTGPPSYDAFNNWGQGTQVTVSS |
| 135. | EPL2 Anti-EPCAM | QVQLQESGGGLVQPGGSLRLSCAASESISSFIAVGWYRQAPGKERELV |
| | sequence | AGINRSGFTYYTDSVKGRFSISRDNAKNTVLLQMTSLKPEDTAVYYCN |
| | | AGGLYFSNAYTQGDYWGQGTQVTVSS |
| 136. | EPL43 Anti-EPCAM | QVQLQESGGGLVQTGGSLRLSCAASESISSFIAVGWYRQAPGKERELV |
| | sequence | AGINRSGFTYYTDSVKGRFSISRDNAKNTVLLQMTSLKPEDTAVYYCN |
| | | AGGLYFSNAYTQGDYWGQGTQVTVSS |
| 137. | EPL10 Anti-EPCAM | QVQLQESGGGLVQAGGSLRLSCAASGSVFRANVMGWYRQAPGKQ |
| | sequence | HELVARIDPGGTTTYADPVKGRFTISRDNAKKTVYLQMNSLKPDDTA |
| | | VYYCNAIILLSGGPKDYWGQGTQVTVSS |
| 138. | EPL49 Anti-EPCAM | QVQLQESGGGLVQAGGSLRLSCAPSGRTSSIFGMGWFRQAPGKERE |
| | sequence | FVASINWSGGSTSYADSVKGRFTISRDNAKNEMYLQMNSLKFEDTAV |
| | | YVCAAAVLTNKPSWNFWGQGTQVTVSS |
| 139. | EPL58 Anti-EPCAM | QVQLQESGGGLVQAGGSLRLSCAASGPIFSDTIRTMGWYRQAAGKQ |
| | sequence | RELVATIASFPSRTNYVDSVKGRFTISRDIAKNTVYLQMDSLKPEDTAV |
| | | YYCNVDLASIPTKTYWGQGTQVTVSS |
| 140. | EPL74 Anti-EPCAM | QVQLQESGGGLVQAGGSLRLSCAASGSIFGINAMGWYRQAPGKQR |
| | sequence | ESVAFITIGGNTNYLDSVKGRFTISRDNAKNTVYLQMNGLKPEDTAVY |
| | | YCNTNPPLILTAGGLYWGQGTQVTVSS |
| 141. | EPL78 Anti-EPCAM | QVQLQESGGGLVQPGGSLRLSCATSANRFNINVMGWYRQAPGQQR |
| | sequence | ELVATINIGGSTDYADSVKGRFTISRDNAKNTVYLQLSDLKPEDTAVYY |
| | | CNVKLRVSGPTGPNVYWGQGTQVTVSS |
| 142. | EPL82 Anti-EPCAM | QVQLQESGGGLVQAGGSLKLSCTASGTILSTMAWYRQAPGKQRELV |
| | sequence | ATISRGGTTNYSDSVKGRFAISRDSTKNTVYLQMNSLKPEDTAVYYCN |
| | | TPLTDYGMGYNWGQGTQVTVSS |
| 143. | EPL83 Anti-EPCAM | QVQLQESGGGLVQAGGSLRLSCAVSGSIFSLNTLAWYRQAPGRQRDL |
| | sequence | IARITGGGTTVYADSVKGRFTISRDNAKNTVYLQMNSLKPEDTAVYYC |
| | | NLMVRHPSGSTYEYWGQGTQVTVSS |
| 144. | EPL97 Anti-EPCAM | QVQLQESGGGLVQAGGSLRLSCAASGIIFRGTTMGWFRQAPGKQRE |
| | sequence | SVASISPLGTTSYSGSVEGRFTVSRDNAKNTLFLQMNSLKSEDTAVYYC |
| | | NAIQVTNVGPRVYWGQGTQVTVSS |
| 145. | EPL109 Anti-EPCAM | QVQLQESGGGLVQPGGSLRLSCASSGFTLDDYTIGWFRQAPGKEREG |
| | sequence | VSCISRRDDSTYYADSVKGRFTISRDNAKNTVDLQMISLRPEDTAVYYC |
| | | AATPRSYTLRCLGKFDFQGQGTQVTVSS |
| 146. | EPL117 Anti-EPCAM | QVQLQESGGGLVQAGGSLRLSCAASGNIVRMTNMAWYRQAPGKQ |
| | sequence | REFVATISAGGSTTYVDSVKDRFTISRDNTKNTVYLQMNYLKPEDTAV |
| | | YYCATGSILTNRGAIPGSWGHGTQVTVSS |
| 147. | EPL127 Anti-EPCAM | QVQLQESGGGLVQAGGSLRLSCAAPGFAFNDHAILWFRQAPGKERE |
| | sequence | GVSEICRDGTTYYTDSVKGRFTISSDNAKNTVYLQMNSVKTDDTAVYY |
| | | CAVDRRRYYCSGNRAFSSDYYYWGQGTQVTVSS |
| 148. | EPL152 Anti-EPCAM | QVQLQESGGGLVQAGGSLRLSCVHSGSIFRASTMAWYRQAPGKQRE |
| | sequence | LVAQIMSGGGTNYAGSVKGRFTISRDNANNTVYLQMNSLKPEDTAV |
| | | YYCNAAQITSWGPKVYWGQGTQVTVSS |
| 149. | EPL189 Anti-EPCAM | QVQLQESGGGLVQPGGSLRLSCAASGRINSINTMGWYRQAPGNQR |
| | sequence | ELVAEITRGGTTNYADSVQGRYAISRDNAKNLVYLQMNSLKPEDTDV |
| | | YYCNAQTFPTFSRPTGLDYWGQGTQVTVSS |
| 150. | EPL90 CDR1 | GFIFRAASMA |
| 151. | EPL118 CDR1 | GFIFRAASMG |
| 152. | EPL138 CDR1 | GFIFRAASMD |
| 153. | EPL145 CDR1 | GFIFRAASMG |
| 154. | EPL164 CDR1 | GFIFRAASMD |
| 155. | EPL31 CDR1 | GDTFLRYAMG |
| 156. | EPL55 CDR1 | GDTFLRYAMG |
| 157. | EPL57 CDR1 | GDTFLRYAMG |
| 158. | EPL136 CDR1 | GDTFLRYAMG |
| 159. | EPL34 CDR1 | GFTFSSYYMS |
| 160. | EPL86 CDR1 | GFTFSDWAMS |
| 161. | EPL153 CDR1 | GFTFSDWAMS |
| 162. | EPL20 CDR1 | GNVFRAATMA |
| 163. | EPL70 CDR1 | GNVFRAATMA |
| 164. | EPL125 CDR1 | GNVFRAATMA |
| 165. | EPL13 CDR1 | GFAFGNHWMY |
| 166. | EPL129 CDR1 | GFAFGNHWMY |
| 167. | EPL159 CDR1 | GFAFGNHWMY |
| 168. | EPL120 CDR1 | GFIFRAASIS |
| 169. | EPL126 CDR1 | GFIFRAASMG |
| 170. | EPL60 CDR1 | EYILSMYRMA |
| 171. | EPL156 CDR1 | EYILSMYRMA |
| 172. | EPL2 CDR1 | ESISSFIAVG |
| 173. | EPL43 CDR1 | ESISSFIAVG |
| 174. | EPL10 CDR1 | GSVFRANVMG |
| 175. | EPL49 CDR1 | GRTSSIFGMG |
| 176. | EPL58 CDR1 | GPIFSDTIRTMG |
| 177. | EPL74 CDR1 | GSIFGINAMG |
| 178. | EPL78 CDR1 | ANRFNINVMG |
| 179. | EPL82 CDR1 | GTILSTMA |
| 180. | EPL83 CDR1 | GSIFSLNTLA |
| 181. | EPL97 CDR1 | GIIFRGTTMG |
| 182. | EPL109 CDR1 | GFTLDDYTIG |
| 183. | EPL117 CDR1 | GNIVRMTNMA |
| 184. | EPL127 CDR1 | GFAFNDHAIL |
| 185. | EPL152 CDR1 | GSIFRASTMA |
| 186. | EPL189 CDR1 | GRINSINTMG |
| 187. | EPL90 CDR2 | SISSGAFTNYADSVKA |
| 188. | EPL118 CDR2 | TVSSGDFTNYADSVKG |
| 189. | EPL138 CDR2 | TISSGGFTNYADSVKG |
| 190. | EPL145 CDR2 | TVSSGGFTNYADSVKG |
| 191. | EPL164 CDR2 | TISSTGFTNYANSVKG |
| 192. | EPL31 CDR2 | AITWNGGNTDYAGSLKG |
| 193. | EPL55 CDR2 | AITWNGGNTDYAGSLKG |
| 194. | EPL57 CDR2 | AITWNGGNTDYADSLKG |
| 195. | EPL136 CDR2 | AITWNGGNTDYAGSLKG |
| 196. | EPL15 CDR2 | GIHYTGDWTNYADSVKG |
| 197. | EPL34 CDR2 | GIHYTGDWTNYADSVKG |
| 198. | EPL86 CDR2 | GIHYGDHTTHYADFVKG |
| 199. | EPL153 CDR2 | SIHYGDHTTHYADFVKG |
| 200. | EPL20 CDR2 | TIASGGTTNYADFVKG |
| 201. | EPL70 CDR2 | TIASGGTTNYADFVKG |
| 202. | EPL125 CDR2 | TIASGGTTNYADFVKG |
| 203. | EPL13 CDR2 | SISSGGSTNYVDSVKG |
| 204. | EPL129 CDR2 | SISSGGSTNYVDSVKG |
| 205. | EPL159 CDR2 | SISSGGSTNYVDSVKG |
| 206. | EPL120 CDR2 | TINSGGFTNYADSVLG |
| 207. | EPL126 CDR2 | TINSGGFTNYADSVKG |
| 208. | EPL60 CDR2 | DMSSGGTTNYADFVKG |
| 209. | EPL156 CDR2 | DMSSGGTTNYADFVKG |
| 210. | EPL2 CDR2 | GINRSGFTYYTDSVKG |
| 211. | EPL43 CDR2 | GINRSGFTYYTDSVKG |
| 212. | EPL10 CDR2 | RIDPGGTTTYADPVKG |
| 213. | EPL49 CDR2 | SINWSGGSTSYADSVKG |
| 214. | EPL58 CDR2 | TIASFPSRTNYVDSVKG |
| 215. | EPL74 CDR2 | FITIGGNTNYLDSVKG |
| 216. | EPL78 CDR2 | TINIGGSTDYADSVKG |
| 217. | EPL82 CDR2 | TISRGGTTNYSDSVKG |
| 218. | EPL83 CDR2 | RITGGGTTVYADSVKG |
| 219. | EPL97 CDR2 | SISPLGTTSYSGSVEG |
| 220. | EPL109 CDR2 | CISRRDDSTYYADSVKG |
| 221. | EPL117 CDR2 | TISAGGSTTYVDSVKD |
| 222. | EPL127 CDR2 | EICRDGTTYYTDSVKG |
| 223. | EPL152 CDR2 | QIMSGGGTNYAGSVKG |
| 224. | EPL189 CDR2 | EITRGGTTNYADSVQG |
| 225. | EPL90 CDR3 | TFLRSDGHHTI |
| 226. | EPL118 CDR3 | TFVRSDGHHTI |
| 227. | EPL138 CDR3 | TFLRSDGHHTI |
| 228. | EPL145 CDR3 | TFVRSDGHHTI |
| 229. | EPL164 CDR3 | TFLRSDGQHSI |
| 230. | EPL31 CDR3 | DLTFGLASSHYQYDY |
| 231. | EPL55 CDR3 | DLTFGLASSHYQYDY |
| 232. | EPL57 CDR3 | DLTFGLASSHYQYDY |
| 233. | EPL136 CDR3 | DLTFGLASSHYQYDY |
| 234. | EPL15 CDR3 | GSD |
| 235. | EPL34 CDR3 | GSD |
| 236. | EPL86 CDR3 | GST |
| 237. | EPL153 CDR3 | GTT |
| 238. | EPL20 CDR3 | GYLTSLGPKNY |
| 239. | EPL70 CDR3 | GYLTSLGPKNY |
| 240. | EPL125 CDR3 | LYLTSLGPKSY |
| 241. | EPL13 CDR3 | SDN |
| 242. | EPL129 CDR3 | SDN |
| 243. | EPL159 CDR3 | SDN |
| 244. | EPL120 CDR3 | TFLRSDGQPPI |
| 245. | EPL126 CDR3 | TFLRSDGQPPI |
| 246. | EPL60 CDR3 | AGRTGPPSYDAFNN |
| 247. | EPL156 CDR3 | AGRTGPPSYDAFNN |
| 248. | EPL2 CDR3 | GGLYFSNAYTQGDY |
| 249. | EPL43 CDR3 | GGLYFSNAYTQGDY |
| 250. | EPL10 CDR3 | IILLSGGPKDY |
| 251. | EPL49 CDR3 | AVLTNKPSWNF |
| 252. | EPL58 CDR3 | DLASIPTKTY |
| 253. | EPL74 CDR3 | NPPLILTAGGLY |
| 254. | EPL78 CDR3 | KLRVSGPTGPNVY |
| 255. | EPL82 CDR3 | PLTDYGMGYN |
| 256. | EPL83 CDR3 | MVRHPSGSTYEY |
| 257. | EPL97 CDR3 | IQVTNVGPRVY |
| 258. | EPL109 CDR3 | TPRSYTLRCLGKFDF |
| 259. | EPL117 CDR3 | GSILTNRGAIPGS |
| 260. | EPL127 CDR3 | DRRRYYCSGNRAFSSDYYY |
| 261. | EPL152 CDR3 | AQITSWGPKVY |
| 262. | EPL189 CDR3 | QTFPTFSRPTGLDY |
| 263. | H13 Anti-human | EVQLVESGGGLVQPGGSLTLSCAASGFAFGNHWMYWYRQAPGRGR |
| | EPCAM sequence | ELVASISSGGSTNYVDSVKGRFTISRDNAKNTLYLQMNSLRAEDTAVY |
| | name | YCGTSDNWGQGTLVTVSS |
| 264. | H138 Anti-human | EVQLLESGGGLVQPGGSLTLSCAASGFIFRAASMDWYRQFPGNERES |
| | EPCAM sequence | IATISSGGFTNYADSVKGRFTISRDNSKNTLYLQMNSLRAEDTAVYYCG |
| | name | ATFLRSDGHHTINGQGTLVTVSS |
| 265. | MMP9 + matriptase | KPLGLQARVV |
| | Cleavable Linker | |
| | Sequence | |
| 266. | MMP9 + matriptase + | PQASTGRSGG |
| | uPA Cleavable Linker | |
| | Sequence | |
| 267. | MMP9 + matriptase + | PQGSTGRAAG |
| | uPA Cleavable Linker | |
| | Sequence | |
| 268. | Matriptase + uPA | PPASSGRAGG |
| | Cleavable Linker | |
| | Sequence | |
| 269. | MMP9 + matriptase | PIPVQGRAH |
| | Cleavable Linker | |
| | Sequence | |
| 270. | L002 Non-cleavable | SGGGGSGGVV |
| | Linker Sequence | |
| 271. | L016 Non-cleavable | SGGGGSGGGGSGGGGS |
| | Linker Sequence | |
| 272. | L017 Non-cleavable | SGGGGSGGGGSGGGGGS |
| | Linker Sequence | |
| 273. | L046 Non-cleavable | SGGGGSGGGS |
| | Linker Sequence | |
| 274. | L001 linker sequence: | KPLGLQARVV |
| | MMP9 + matriptase | |
| 275. | L040 linker sequence: | PQASTGRSGG |
| | MMP9 + matriptase + | |
| | uPA | |
| 276. | L1041 linker | PQGSTGRAAG |
| | sequence: MMP9 + | |
| | matriptase + uPA | |
| 277. | L042 linker sequence: | PPASSGRAGG |
| | matriptase + uPA | |
| 278. | L045 linker sequence: | PIPVQGRAH |
| | MMP9 + matriptase | |
| 279. | L043 linker sequence: | PQGSTARSAG |
| | MMP9 + matriptase + | |
| | uPA | |

What is claimed is:

1. A conditionally active chimeric antigen receptor that comprises a single polypeptide chain, comprising
(a) a binding moiety comprising a non-CDR loop and a cleavable linker;
(b) a target antigen binding domain;
(c) a transmembrane domain; and
(d) an intracellular signaling domain;
wherein the binding moiety is capable of masking the binding of the target antigen binding domain to its target.

2. The conditionally active chimeric antigen receptor of claim 1, wherein the binding moiety is a natural peptide, a synthetic peptide, or an engineered scaffold.

3. The conditionally active chimeric antigen receptor of claim 2, wherein the engineered scaffold is a sdAb, a scFv, a Fab, a VHH, a fibronectin type III domain, immunoglobulin-like scaffold, DARPin, cystine knot peptide, lipocalin, three-helix bundle scaffold, protein G-related albumin-binding module, or a DNA or RNA aptamer scaffold.

4. The conditionally active chimeric antigen receptor of any one of claim 1-3, wherein the non-CDR-loop comprises a non-CDR loop of a variable domain, a constant domain, a C1-set domain, a C2-set domain, an I-domain, or a combination thereof.

5. The conditionally active chimeric antigen receptor of any one of claims 1-4, wherein the binding moiety further comprises one or more complementarity determining regions (CDRs).

6. The conditionally active chimeric antigen receptor of any one of claims 1-5, wherein the binding moiety is capable of binding an antigen, modifying a tumor microenvironment, activating an immune cell, or a combination thereof.

7. The conditionally active chimeric antigen receptor of claim 5 or 6, wherein the CDR loop provides a binding site specific for an antigen.

8. The conditionally active chimeric antigen receptor of claim 6 or 7, wherein the antigen is a tumor antigen.

9. The conditionally active chimeric antigen receptor of claim 8, wherein the tumor antigen comprises EGFR, PSMA, EpCAM, BCMA, 5T4, AFP, Ax1, B7-H3, Cadherin-6, CAIX, CD117, CD123, CD138, CD166, CD19, CD20, CD205, CD22, CD30, CD33, CD352, CD37, CD38, CD44, CD52, CD56, CD70, CD71, CD74, CD79b, CEACAM5, c-MET, DLL3, EphA2, FAP, FGFR2, FGFR3, glypican-3, FLT-3, FOLR1, gpNMB, HER2, HPV-16 E6, HPV-16 E7, ITGA3, SLC39A6, Mesothelin, Muc1, Muc16, NaPi2b, Nectin-4, P-cadherin, Prolactin R, PSCA, PTK7, ROR1, SLC44A4, SLTRK5, SLTRK6, STEAP1, TIM1, Trop2, or WT1.

10. The conditionally active chimeric antigen receptor of claim 6 or 7, wherein the antigen comprises PD1 or CTLA4.

11. The conditionally active chimeric antigen receptor of any one of claims 1-5, wherein the binding moiety comprises a non-immunoglobulin molecule.

12. The conditionally active chimeric antigen receptor of claim 11, wherein the non-immunoglobulin molecule comprises a natural peptide, a synthetic peptide, an engineered scaffold, or an engineered bulk serum protein.

13. The conditionally active chimeric antigen receptor of any one of claims 1-12, wherein the intracellular signaling domain comprises a signaling domain from ZAP70, CD3 zeta, CD28, or 4-1BB.

14. The conditionally active chimeric antigen receptor of any one of claims 1-13, wherein the cleavable linker comprises a cleavage site.

15. The conditionally active chimeric antigen receptor of claim 14, wherein the cleavage site is recognized by a protease, is pH sensitive, or is cleaved by chemical degradation.

16. The conditionally active chimeric antigen receptor of any one of claims 1-15, wherein the binding moiety is bound to the target antigen binding domain.

17. The conditionally active chimeric antigen receptor of claim 16, wherein the binding moiety is covalently linked to the target antigen binding domain.

18. The conditionally active chimeric antigen receptor of claim 16, wherein the binding moiety is capable of masking the binding of the target antigen binding domain to its target via specific intermolecular interactions between the binding moiety and the target antigen binding domain.

19. The conditionally active chimeric antigen receptor of claim 18, wherein the non-CDR loop provides a binding site specific for binding of the moiety to the target antigen binding domain.

20. The conditionally active chimeric antigen receptor of any one of claims 1-19, wherein upon cleavage of the cleavage site, the binding moiety is separated from the target antigen binding domain and the target antigen binding domain binds to its target.

21. The conditionally active chimeric antigen receptor of any one of claims 1-20, wherein the target antigen binding domain binds to a tumor antigen.

22. The conditionally active chimeric antigen receptor of claim 21, wherein the tumor antigen comprises EGFR, PSMA, EpCAM, BCMA, 5T4, AFP, Ax1, B7-H3, Cadherin-6, CAIX, CD117, CD123, CD138, CD166, CD19, CD20, CD205, CD22, CD30, CD33, CD352, CD37, CD38, CD44, CD52, CD56, CD70, CD71, CD74, CD79b, CEACAM5, c-MET, DLL3, EphA2, FAP, FGFR2, FGFR3, glypican-3, FLT-3, FOLR1, gpNMB, HER2, HPV-16 E6, HPV-16 E7, ITGA3, SLC39A6, Mesothelin, Muc1, Muc16, NaPi2b, Nectin-4, P-cadherin, Prolactin R, PSCA, PTK7, ROR1, SLC44A4, SLTRK5, SLTRK6, STEAP1, TIM1, Trop2, or WT1.

23. The conditionally active chimeric antigen receptor of any one of claims 1-22, wherein the target antigen binding domain binds to an immune checkpoint protein.

24. The conditionally active chimeric antigen receptor of claim 23, wherein the immune checkpoint protein comprises CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA.

25. The conditionally active chimeric antigen receptor of any one of claims 14-24, wherein the cleavage site is recognized by a serine protease, a cysteine protease, an aspartate protease, a threonine protease, a glutamic acid protease, a metalloproteinase, a gelatinase, or a asparagine peptide lyase.

26. The conditionally active chimeric antigen receptor of any one of claims 14-24, wherein the cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elatase, a cathepsin G, a proteinase 3, a neutrophil serine protease 4, a mast cell chymase, a mast cell tryptase, a dipeptidyl peptidase, or a dipeptidyl peptidase IV (DPPIV/CD26).

27. The conditionally active chimeric antigen receptor of any one of claims 1-26, wherein the target antigen binding domains comprises an sdAb, a scFv, a Fab, a variable heavy chain domain (VHH), or a combination thereof.

28. A conditionally active T-cell receptor fusion protein comprising a single polypeptide chain comprising:
(a) a T-cell receptor subunit comprising:
(i) at least a portion of a T-cell receptor extracellular domain;
(ii) a transmembrane domain; and
(iii) a T-cell receptor intracellular domain comprising a stimulatory domain from an intracellular signaling domain;
(b) a binding moiety comprising a non-CDR loop and a cleavable linker; and
(c) a target antigen binding domain;
wherein the binding moiety is capable of masking the binding of the target antigen binding domain to its target.

29. The conditionally active T-cell receptor fusion protein of claim 28, further comprising a costimulatory domain, wherein the costimulatory domain is a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

30. The conditionally active T-cell receptor fusion protein of claim 29, wherein the at least one but not more than 20 modifications thereto comprises a modification of an amino acid that mediates cell signaling or a modification of an amino acid that is phosphorylated in response to a ligand binding to the encoded T-cell receptor fusion protein.

31. The conditionally active T-cell receptor fusion protein of any one of claims 28-30, wherein the encoded transmembrane domain comprises a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CDS, CD8, CD9, CD16, CD22, CD33, CD28, CD37, CD64, CD80, CD86, CD134, CD137, CD154, functional fragments thereof, and amino acid sequences thereof having at least one but not more than 20 modifications thereto.

32. The conditionally active T-cell receptor fusion protein of claim any one of claims 28-31, wherein the T-cell receptor fusion comprises an immunoreceptor tyrosine-based activation motif (ITAM) or portion thereof, wherein the ITAM or portion thereof is from a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b 1 chain, Fc gamma receptor 2b2 chain, Fc gamma receptor 3a chain, Fc gamma receptor 3b chain, Fc beta receptor 1 chain, TYROBP (DAP12), CDS, CD16a, CD16b, CD22, CD23, CD32, CD64, CD79a, CD79b, CD89, CD278, CD66d, functional fragments thereof, and amino acid sequences thereof having at least one but not more than 20 modifications thereto.

33. The conditionally active T-cell receptor fusion protein of any one of claims 28-32, wherein the T-cell receptor intracellular domain is derived from CD3 epsilon CD3 gamma, CD3 delta, CD3 alpha, CD3 beta, or a combination thereof.

34. The conditionally active T-cell receptor fusion protein of any one of claims 28-33, wherein the T-cell receptor subunit and the target antigen binding domain are operatively linked.

35. The conditionally active T-cell receptor fusion protein of any one of claims 28-34, wherein the T-cell receptor fusion protein incorporates into a T-cell receptor when expressed in a T-cell.

36. The conditionally active T-cell receptor fusion protein of any one of claims 28-35, wherein the antigen binding domain comprises a domain from a human or humanized antibody.

37. The conditionally active T-cell receptor fusion protein of any one of claims 28-36, wherein the binding moiety comprises a natural peptide, a synthetic peptide, or an engineered scaffold.

38. The conditionally active T-cell receptor fusion protein of claim 37, wherein the engineered scaffold comprises a sdAb, a scFv, a Fab, a VHH, a fibronectin type III domain, an immunoglobulin-like scaffold, a DARPin, a cystine knot peptide, a lipocalin, or a three-helix bundle scaffold.

39. The conditionally active T-cell receptor fusion protein of any one of claims 28-38, wherein the non-CDR-loop comprises a non-CDR loop of a variable domain, a constant domain, a C1-set domain, a C2-set domain, an I-domain, or any combinations thereof.

40. The conditionally active T-cell receptor fusion protein of any one of claims 28-39, wherein the binding moiety further comprises complementarity determining regions (CDRs).

41. The conditionally active T-cell receptor fusion protein of any one of claims 28-40, wherein the binding moiety is capable of binding an antigen, modifying a tumor microenvironment, activating an immune cell, or any combinations thereof.

42. The conditionally active T-cell receptor fusion protein of claim 40 or 41, wherein the CDR loop provides a binding site specific for an antigen.

43. The conditionally active T-cell receptor fusion protein of claim 41 or 42, wherein the antigen comprises a tumor antigen.

44. The conditionally active T-cell receptor fusion protein of claim 43, wherein the tumor antigen comprises EGFR, PSMA, EpCAM, BCMA, 5T4, AFP, Ax1, B7-H3, Cadherin-6, CAIX, CD117, CD123, CD138, CD166, CD19, CD20, CD205, CD22, CD30, CD33, CD352, CD37, CD38, CD44, CD52, CD56, CD70, CD71, CD74, CD79b, CEACAM5, c-MET, DLL3, EphA2, FAP, FGFR2, FGFR3, glypican-3, FLT-3, FOLR1, gpNMB, HER2, HPV-16 E6, HPV-16 E7, ITGA3, SLC39A6, Mesothelin, Muc1, Muc16, NaPi2b, Nectin-4, P-cadherin, Prolactin R, PSCA, PTK7, ROR1, SLC44A4, SLTRK5, SLTRK6, STEAP1, TIM1, Trop2, or WT1.

45. The conditionally active T-cell receptor fusion protein of claim 41 or 42, wherein the antigen comprises PD1 or CTLA4.

46. The conditionally active T-cell receptor fusion protein of any one of claims 28-45, wherein the intracellular signaling domain comprises a signaling domain from ZAP70, CD3 zeta, CD28, or 4-1BB.

47. The conditionally active T-cell receptor fusion protein of any one of claims 28-46, wherein the cleavable linker comprises a cleavage site.

48. The conditionally active T-cell receptor fusion protein of claim 47, wherein the cleavage site is recognized by a protease, is pH sensitive, or is cleaved by chemical degradation.

49. The conditionally active T-cell receptor fusion protein of any one of claims 28-48, wherein the binding moiety is bound to the target antigen binding domain.

50. The conditionally active T-cell receptor fusion protein of claim 49, wherein the binding moiety is covalently linked to the target antigen binding domain.

51. The conditionally active T-cell receptor fusion protein of claim 49, wherein the binding moiety is capable of masking the binding of the target antigen binding domain to its target via specific intermolecular interactions between the binding moiety and the target antigen binding domain.

52. The conditionally active T-cell receptor fusion protein of claim 51, wherein the non-CDR loop provides a binding site specific for binding of the moiety to the target antigen binding domain.

53. The conditionally active T-cell receptor fusion protein of any one of claims 47-52, wherein upon cleavage of the cleavage site, the binding moiety is separated from the target antigen binding domain and the target antigen binding domain binds to its target.

54. The conditionally active T-cell receptor fusion protein of any one of claims 28-53, wherein the target antigen binding domain binds to a tumor antigen.

55. The conditionally active T-cell receptor fusion protein of claim 54, wherein the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1.

56. The conditionally active T-cell receptor fusion protein of any one of claims 28-53, wherein the target antigen binding domain binds to an immune checkpoint protein.

57. The conditionally active T-cell receptor fusion protein of claim 56, wherein the immune checkpoint protein is CD27, CD137, 2B4, TIGIT, CD155, ICOS, HVEM, CD40L, LIGHT, TIM-1, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA.

58. The conditionally active T-cell receptor fusion protein of any one of claims 47-57, wherein the cleavage site is recognized by a serine protease, a cysteine protease, an aspartate protease, a threonine protease, a glutamic acid protease, a metalloproteinase, a gelatinase, or a asparagine peptide lyase.

59. The conditionally active T-cell receptor fusion protein of any one of claims 47-57, wherein the cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β0 converting enzyme, a thrombin, a FAP (FAP-α), a type II transmembrane serine protease (TTSP), a neutrophil elatase, a cathepsin G, a proteinase 3, a neutrophil serine protease 4, a mast cell chymase, a mast cell tryptase, a dipeptidyl peptidase, or a dipeptidyl peptidase IV (DPPIV/CD26).

60. The conditionally active T-cell receptor fusion protein of any one of claims 28-59, wherein the target antigen binding domains comprises an sdAb, a scFv, a Fab, a variable heavy chain domain (VHH), or a combination thereof.

61. A conditionally active T-cell receptor comprising a binding moiety comprising a a non-CDR loop and a cleavable linker, wherein the binding moiety is capable of masking the binding of the T-cell receptor to its target.

62. The conditionally active T-cell receptor of claim 61, wherein the non-CDR loop of the binding moiety provides a binding site specific for T-cell receptor alpha, T-cell receptor beta, or a combination thereof.

63. The conditionally active T-cell receptor of claim 61 or 62, wherein the binding moiety is a natural peptide or, a synthetic peptide, an engineered scaffold.

64. The conditionally active T-cell receptor of claim 63, wherein the engineered scaffold comprises a sdAb, a scFv, a Fab, a VHH, a fibronectin type III domain, an immunoglobulin-like scaffold, a DARPin, a cystine knot peptide, a lipocalin, or a three-helix bundle scaffold.

65. The conditionally active T-cell receptor of any one of claims 61-64, wherein the non-CDR-loop comprises a non-CDR loop of a variable domain, a constant domain, a C1-set domain, a C2-set domain, an I-domain, or any combinations thereof.

66. The conditionally active T-cell receptor of any one of claims 61-65, wherein the binding moiety further comprises complementarity determining regions (CDRs).

67. The conditionally active T-cell receptor of any one of claims 61-66, wherein the binding moiety is capable of binding an antigen, modifying a tumor microenvironment, activating an immune cell, or any combinations thereof.

68. The conditionally active T-cell receptor of claim 66 or 67, wherein the CDR loop provides a binding site specific for an antigen.

69. The conditionally active T-cell receptor of claim 67 or 68, wherein the antigen comprises a tumor antigen.

70. The conditionally active T-cell receptor of claim 69, wherein the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1.

71. The conditionally active T-cell receptor of claim 67 or 68, wherein the antigen comprises PD1 or CTLA4.

72. The conditionally active T-cell receptor of any one of claims 61-71, wherein the intracellular signaling domain comprises a signaling domain from ZAP70, CD3 zeta, CD28, or 4-1BB.

73. The conditionally active T-cell receptor of any one of claims 61-72, wherein the cleavable linker comprises a cleavage site.

74. The conditionally active T-cell receptor of claim 73, wherein the cleavage site is recognized by a protease, is pH sensitive, or is cleaved by chemical degradation.

75. The conditionally active T-cell receptor of any one of claims 61-74, wherein the binding moiety is bound to the target antigen binding domain.

76. The conditionally active T-cell receptor of claim 75, wherein the binding moiety is covalently linked to the target antigen binding domain.

77. The conditionally active T-cell receptor of claim 75, wherein the binding moiety is capable of masking the binding of the target antigen binding domain to its target via specific intermolecular interactions between the binding moiety and the target antigen binding domain.

78. The conditionally active T-cell receptor of claim 77, wherein the non-CDR loop provides a binding site specific for binding of the moiety to the target antigen binding domain.

79. The conditionally active T-cell receptor of any one of claims 61-78, wherein upon cleavage of the cleavage site, the binding moiety is separated from the target antigen binding domain and the target antigen binding domain binds to its target.

80. The conditionally active T-cell receptor of any one of claims 61-79, wherein the target antigen binding domain binds to a tumor antigen.

81. The conditionally active T-cell receptor of claim 80, wherein the tumor antigen comprises EpCAM, EGFR, HER-2, HER-3, c-Met, FoIR, PSMA, CD38, BCMA, CEA, 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1.

82. The conditionally active T-cell receptor of any one of claims 61-81, wherein the target antigen binding domain binds to an immune checkpoint protein.

83. The conditionally active T-cell receptor of claim 82, wherein the immune checkpoint protein comprises CD27, CD137, 2B4, TIGIT, CD155, ICOS, HVEM, CD40L, LIGHT, TIM-1, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA.

84. The conditionally active T-cell receptor of any one of claims 61-83, wherein the protease cleavage site is recognized by a serine protease, a cysteine protease, an aspartate protease, a threonine protease, a glutamic acid protease, a metalloproteinase, a gelatinase, or a asparagine peptide lyase.

85. The conditionally active T-cell receptor of any one of claims 61-83, wherein the protease cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elatase, a cathepsin G, a proteinase 3, a neutrophil serine protease 4, a mast cell chymase, a mast cell tryptase, a dipeptidyl peptidase, or a dipeptidyl peptidase IV (DPPIV/CD26).

86. The conditionally active T-cell receptor of any one of claims 61-86, wherein the target antigen binding domains comprises an sdAb, a scFv, a Fab, a variable heavy chain domain (VHH), or a combination thereof.


SEQUENCE LISTING (292 sequences)

SEQ ID NO: 1 - protein, 8 aa, Unknown
  Description of Unknown Protease cleavage site sequence
KRALGLPG

SEQ ID NO: 2 - protein, 40 aa, Unknown
  Description of Unknown Protease cleavage site sequence
DEDEDEDEDEDEDEDERPLALWRSDRDRDRDRDRDRDRDR

SEQ ID NO: 3 - protein, 5 aa, Unknown
  Description of Unknown Protease cleavage site sequence
  MOD_RES (3)..(3): Ser or Thr
  MOD_RES (4)..(4): Leu or Ile
  MOD_RES (5)..(5): Ser or Thr
PRXXX

SEQ ID NO: 4 - protein, 5 aa, Unknown
  Description of Unknown Protease cleavage site sequence
LEATA

SEQ ID NO: 5 - protein, 10 aa, Unknown
  Description of Unknown Protease cleavage site sequence
GGAANLVRGG

SEQ ID NO: 6 - protein, 10 aa, Unknown
  Description of Unknown Protease cleavage site sequence
SGRIGFLRTA

SEQ ID NO: 7 - protein, 6 aa, Unknown
  Description of Unknown Protease cleavage site sequence
PLGLAG

SEQ ID NO: 8 - protein, 6 aa, Unknown
  Description of Unknown Protease cleavage site sequence
  MOD_RES (6)..(6): Any amino acid
PLGLAX

SEQ ID NO: 9 - protein, 6 aa, Unknown
  Description of Unknown Protease cleavage site sequence
PLGCAG

SEQ ID NO: 10 - protein, 8 aa, Unknown
  Description of Unknown Protease cleavage site sequence
ESPAYYTA

SEQ ID NO: 11 - protein, 6 aa, Unknown
  Description of Unknown Protease cleavage site sequence
RLQLKL

SEQ ID NO: 12 - protein, 7 aa, Unknown
  Description of Unknown Protease cleavage site sequence
RLQLKAC

SEQ ID NO: 13 - protein, 7 aa, Unknown
  Description of Unknown Protease cleavage site sequence
  MOD_RES (3)..(3): Cit
  MOD_RES (5)..(5): Hof
EPXGXYL

SEQ ID NO: 14 - protein, 5 aa, Unknown
  Description of Unknown Protease cleavage site sequence
SGRSA

SEQ ID NO: 15 - protein, 4 aa, Unknown
  Description of Unknown Protease cleavage site sequence
DAFK

SEQ ID NO: 16 - protein, 5 aa, Unknown
  Description of Unknown Protease cleavage site sequence
GGGRR

SEQ ID NO: 17 - protein, 4 aa, Unknown
  Description of Unknown Protease cleavage site sequence
GFLG

SEQ ID NO: 18 - protein, 4 aa, Unknown
  Description of Unknown Protease cleavage site sequence
ALAL

SEQ ID NO: 19 - protein, 2 aa, Unknown
  Description of Unknown Protease cleavage site sequence
FK

SEQ ID NO: 20 - protein, 3 aa, Unknown
  Description of Unknown Protease cleavage site sequence
NLL

SEQ ID NO: 21 - protein, 5 aa, Unknown
  Description of Unknown Protease cleavage site sequence
PICFF

SEQ ID NO: 22 - protein, 8 aa, Unknown
  Description of Unknown Protease cleavage site sequence
GGPRGLPG

SEQ ID NO: 23 - protein, 6 aa, Unknown
  Description of Unknown Protease cleavage site sequence
HSSKLQ

SEQ ID NO: 24 - protein, 7 aa, Unknown
  Description of Unknown Protease cleavage site sequence
HSSKLQL

SEQ ID NO: 25 - protein, 9 aa, Unknown
  Description of Unknown Protease cleavage site sequence
HSSKLQEDA

SEQ ID NO: 26 - protein, 10 aa, Unknown
  Description of Unknown Protease cleavage site sequence
LVLASSSFGY

SEQ ID NO: 27 - protein, 10 aa, Unknown
  Description of Unknown Protease cleavage site sequence
GVSQNYPIVG

SEQ ID NO: 28 - protein, 10 aa, Unknown
  Description of Unknown Protease cleavage site sequence
GVVQASCRLA

SEQ ID NO: 29 - protein, 4 aa, Unknown
  Description of Unknown Protease cleavage site sequence
  MOD_RES (2)..(2): Pip
FXRS

SEQ ID NO: 30 - protein, 6 aa, Unknown
  Description of Unknown Protease cleavage site sequence
DPRSFL

SEQ ID NO: 31 - protein, 6 aa, Unknown
  Description of Unknown Protease cleavage site sequence
PPRSFL

SEQ ID NO: 32 - protein, 4 aa, Unknown
  Description of Unknown Protease cleavage site sequence
DEVD

SEQ ID NO: 33 - protein, 5 aa, Unknown
  Description of Unknown Protease cleavage site sequence
DEVDP

SEQ ID NO: 34 - protein, 8 aa, Unknown
  Description of Unknown Protease cleavage site sequence
KGSGDVEG

SEQ ID NO: 35 - protein, 6 aa, Unknown
  Description of Unknown Protease cleavage site sequence
GWEHDG

SEQ ID NO: 36 - protein, 7 aa, Unknown
  Description of Unknown Protease cleavage site sequence
EDDDDKA

SEQ ID NO: 37 - protein, 9 aa, Unknown
  Description of Unknown Protease cleavage site sequence
KQEQNPGST

SEQ ID NO: 38 - protein, 6 aa, Unknown
  Description of Unknown Protease cleavage site sequence
GKAFRR

SEQ ID NO: 39 - protein, 4 aa, Unknown
  Description of Unknown Protease cleavage site sequence
DAFK

SEQ ID NO: 40 - protein, 4 aa, Unknown
  Description of Unknown Protease cleavage site sequence
DVLK

SEQ ID NO: 41 - protein, 4 aa, Unknown
  Description of Unknown Protease cleavage site sequence
DAFK

SEQ ID NO: 42 - protein, 7 aa, Unknown
  Description of Unknown Protease cleavage site sequence
ALLLALL

SEQ ID NO: 43 - protein, 253 aa, Artificial Sequence
  Description of Artificial Sequence Synthetic polypeptide
MALPVTALLLPLALLLHAARPTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 377 aa, Artificial Sequence
  Description of Artificial Sequence Synthetic polypeptide
MALPVTALLLPLALLLHAARPEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 509 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGSGGSGGGGSGGGGGSGEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 514 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQANPWVPPREGLEWVSSISGSGRDTLYADSVKGRFTISRDNAKTTLYLQMNSLRPEDTAVYYCTIGGSLSVSSQGTLVTVSSGSGGSGGGGSGGGGGSGEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 514 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQANPWVPPREGLEWVSSISGSGRDTLYADSVKGRFTISRDNAKTTLYLQMNSLRPEDTAVYYCTIGGSLSVSSQGTLVTVSSGGGGLSGRSDNHGGGGTEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 378 aa, Artificial Sequence
  Description of Artificial Sequence Synthetic polypeptide
MALPVTALLLPLALLLHAARPEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 510 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGSGGSGGGGSGGGGGSGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 514 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQAHEPLTCRGLEWVSSISGSGRDTLYADSVKGRFTISRDNAKTTLYLQMNSLRPEDTAVYYCTIGGSLSVSSQGTLVTVSSGSGGSGGGGSGGGGGSGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 514 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQASHPMSCRGLEWVSSISGSGRDTLYADSVKGRFTISRDNAKTTLYLQMNSLRPEDTAVYYCTIGGSLSVSSQGTLVTVSSGSGGSGGGGSGGGGGSGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 514 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQAHEPLTCRGLEWVSSISGSGRDTLYADSVKGRFTISRDNAKTTLYLQMNSLRPEDTAVYYCTIGGSLSVSSQGTLVTVSSGGGGLSGRSDNHGGGGTEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 514 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQASHPMSCRGLEWVSSISGSGRDTLYADSVKGRFTISRDNAKTTLYLQMNSLRPEDTAVYYCTIGGSLSVSSQGTLVTVSSGSGGPLGLQARVVGGSGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 514 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQASHPMSCRGLEWVSSISGSGRDTLYADSVKGRFTISRDNAKTTLYLQMNSLRPEDTAVYYCTIGGSLSVSSQGTLVTVSSGGGGLSGRSDNHGGGGTEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 21 aa, Unknown
  Description of Unknown CD8a signal peptide
MALPVTALLLPLALLLHAARP

SEQ ID NO: 56 - protein, 115 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSS

SEQ ID NO: 57 - protein, 120 aa, Artificial Sequence
  Description of Artificial Sequence Synthetic polypeptide
EVQLVESGGGLVQPGNSLRLSCAASGFTFSKFGMSWVRQANPWVPPREGLEWVSSISGSGRDTLYADSVKGRFTISRDNAKTTLYLQMNSLRPEDTAVYYCTIGGSLSVSSQGTLVTVSS

SEQ ID NO: 58 - protein, 119 aa, Artificial Sequence
  Description of Artificial Sequence Synthetic polypeptide
EVQLVESGGGLVQPGNSLRLSCAASGFTFSKFGMSWVRQAHEPLTCRGLEWVSSISGSGRDTLYADSVKGRFTISRDNAKTTLYLQMNSLRPEDTAVYYCTIGGSLSVSSQGTLVTVSS

SEQ ID NO: 59 - protein, 119 aa, Artificial Sequence
  Description of Artificial Sequence Synthetic polypeptide
EVQLVESGGGLVQPGNSLRLSCAASGFTFSKFGMSWVRQASHPMSCRGLEWVSSISGSGRDTLYADSVKGRFTISRDNAKTTLYLQMNSLRPEDTAVYYCTIGGSLSVSSQGTLVTVSS

SEQ ID NO: 60 - protein, 124 aa, Artificial Sequence
  Description of Artificial Sequence Synthetic polypeptide
EVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSS

SEQ ID NO: 61 - protein, 125 aa, Artificial Sequence
  Description of Artificial Sequence Synthetic polypeptide
EVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSS

SEQ ID NO: 62 - protein, 68 aa, Unknown
  Description of Unknown CD8a hinge/TM sequence
TTTPAPRPPTPAPTIASQPLSLRPEACRPAAGGAVHTRGLDFACDIYIWAPLAGTCGVLLLSLVITLY

SEQ ID NO: 63 - protein, 42 aa, Unknown
  Description of Unknown 4-1BB intracellular domain sequence
KRGRKKLLYIFKQPFMRPVQTTQEEDGCSCRFPEEEEGGCEL

SEQ ID NO: 64 - protein, 112 aa, Unknown
  Description of Unknown CD3z intracellular domain sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 17 aa, Artificial Sequence
  Description of Artificial Sequence Synthetic peptide
GSGGSGGGGSGGGGGSG

SEQ ID NO: 66 - protein, 17 aa, Artificial Sequence
  Description of Artificial Sequence Synthetic peptide
GGGGLSGRSDNHGGGGT

SEQ ID NO: 67 - protein, 17 aa, Artificial Sequence
  Description of Artificial Sequence Synthetic peptide
GSGGPLGLQARVVGGSG

SEQ ID NO: 68 - protein, 10 aa, Artificial Sequence
  Description of Artificial Sequence Synthetic peptide
ANPWVPPREG

SEQ ID NO: 69 - protein, 9 aa, Artificial Sequence
  Description of Artificial Sequence Synthetic peptide
AHEPLTCRG

SEQ ID NO: 70 - protein, 9 aa, Artificial Sequence
  Description of Artificial Sequence Synthetic peptide
ASHPMSCRG

SEQ ID NO: 71 - protein, 516 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQANPWVPPREGLEWVSSISGSGRDTLYADSVKGRFTISRDNAKTTLYLQMNSLRPEDTAVYYCTIGGSLSVSSQGTLVTVSSGGGGPPASSGRAGGGGGGTEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 377 aa, Artificial Sequence
  Description of Artificial Sequence Synthetic polypeptide
MALPVTALLLPLALLLHAARPEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFASDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 514 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQANPWVPPREGLEWVSSISGSGRDTLYADSVKGRFTISRDNAKTTLYLQMNSLRPEDTAVYYCTIGGSLSVSSQGTLVTVSSGGGGLSGRSDNHGGGGTEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFASDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 516 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQANPWVPPREGLEWVSSISGSGRDTLYADSVKGRFTISRDNAKTTLYLQMNSLRPEDTAVYYCTIGGSLSVSSQGTLVTVSSGGGGPPASSGRAGGGGGGTEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFASDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 514 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQANPWVPPREGLEWVSSISGSGRDTLYADSVKGRFTISRDNAKTTLYLQMNSLRPEDTAVYYCTIGGSLSVSSQGTLVTVSSGSGGSGGGGSGGGGGSGEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFASDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 368 aa, Artificial Sequence
  Description of Artificial Sequence Synthetic polypeptide
MALPVTALLLPLALLLHAARPQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 372 aa, Artificial Sequence
  Description of Artificial Sequence Synthetic polypeptide
MALPVTALLLPLALLLHAARPEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 504 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGSGGSGGGGSGGGGGSGEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 511 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGSGGSGGGGSGGGGGSGEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 509 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQQAYWWGNSRDLEWVSSISGSGRDTLYADSVKGRFTISRDNAKTTLYLQMNSLRPEDTAVYYCTIGGSLSVSSQGTLVTVSSGSGGSGGGGSGGGGGSGEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 511 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQQAYWWGNSRDLEWVSSISGSGRDTLYADSVKGRFTISRDNAKTTLYLQMNSLRPEDTAVYYCTIGGSLSVSSQGTLVTVSSGGGGPQGSTGRAAGGGGGTEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 513 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGGGGPQGSTGRAAGGGGGTEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 68 aa, Artificial Sequence
  Description of Artificial Sequence Synthetic polypeptide
TTTPAPRPPTPAPTIASQPLSLRPEACRPAAGGAVHTRGLDFASDIYIWAPLAGTCGVLLLSLVITLY

SEQ ID NO: 84 - protein, 19 aa, Artificial Sequence
  Description of Artificial Sequence Synthetic peptide
GGGGPPASSGRAGGGGGGT

SEQ ID NO: 85 - protein, 122 aa, Artificial Sequence
  Description of Artificial Sequence Synthetic polypeptide
EVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSS

SEQ ID NO: 86 - protein, 120 aa, Artificial Sequence
  Description of Artificial Sequence Synthetic polypeptide
EVQLVESGGGLVQPGNSLRLSCAASGFTFSKFGMSWVRQQAYWWGNSRDLEWVSSISGSGRDTLYADSVKGRFTISRDNAKTTLYLQMNSLRPEDTAVYYCTIGGSLSVSSQGTLVTVSS

SEQ ID NO: 87 - protein, 119 aa, Artificial Sequence
  Description of Artificial Sequence Synthetic polypeptide
EVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSS

SEQ ID NO: 88 - protein, 115 aa, Artificial Sequence [sequence omitted]

SEQ ID NO: 89 - protein, 12 aa, Artificial Sequence
  Description of Artificial Sequence Synthetic peptide
RQSTFMAMDMPI

SEQ ID NO: 90 - protein, 10 aa, Artificial Sequence
  Description of Artificial Sequence Synthetic peptide
QAYWWGNSRD

SEQ ID NO: 91 - protein, 8 aa, Unknown
  Description of Unknown metalloproteinase cleavage site
GPLGIAGQ

SEQ ID NO: 92 - protein, 8 aa, Unknown
  Description of Unknown metalloproteinase cleavage site
AVRWLLTA

SEQ ID NO: 93 - protein, 6 aa, Unknown
  Description of Unknown furin cleavage site
RRRRRR

SEQ ID NO: 94 - protein, 24 aa, Artificial Sequence
  Description of Artificial Sequence Synthetic peptide
IYIWAPLAGTCGVLLLSLVITLYC

SEQ ID NO: 95 - protein, 22 aa, Artificial Sequence
  Description of Artificial Sequence Synthetic peptide
GLLVAGVLVLLVSLGVAIHLCC

SEQ ID NO: 96 - protein, 25 aa, Artificial Sequence
  Description of Artificial Sequence Synthetic peptide
ALIVLGGVAGLLLFIGLGIFFCVRC

SEQ ID NO: 97 - protein, 23 aa, Artificial Sequence
  Description of Artificial Sequence Synthetic peptide
LCYLLDGILFIYGVILTALFLRV

SEQ ID NO: 98 - protein, 26 aa, Artificial Sequence
  Description of Artificial Sequence Synthetic peptide
WVLVVVGGVLACYSLLVTVAFIIFWV

SEQ ID NO: 99 - protein, 25 aa, Artificial Sequence
  Description of Artificial Sequence Synthetic peptide
AAILGLGLVLGLLGPLAILLALYLL

SEQ ID NO: 100 - protein, 24 aa, Artificial Sequence
  Description of Artificial Sequence Synthetic peptide
ALPAALAVISFLLGLGLGVACVLA

SEQ ID NO: 101 - protein, 15 aa, Artificial Sequence
  Description of Artificial Sequence Synthetic peptide
CPEPKSCDTPPPCPR

SEQ ID NO: 102 - protein, 12 aa, Artificial Sequence
  Description of Artificial Sequence Synthetic peptide
ELKTPLGDTTHT

SEQ ID NO: 103 - protein, 7 aa, Artificial Sequence
  Description of Artificial Sequence Synthetic peptide
KCCVDCP

SEQ ID NO: 104 - protein, 7 aa, Artificial Sequence
  Description of Artificial Sequence Synthetic peptide
KYGPPCP

SEQ ID NO: 105 - protein, 15 aa, Homo sapiens
EPKSCDKTHTCPPCP

SEQ ID NO: 106 - protein, 12 aa, Homo sapiens
ERKCCVECPPCP

SEQ ID NO: 107 - protein, 17 aa, Homo sapiens
ELKTPLGDTTHTCPRCP

SEQ ID NO: 108 - protein, 12 aa, Homo sapiens
SPNMVPHAHHAQ

SEQ ID NO: 109 - protein, 15 aa, Artificial Sequence
  Description of Artificial Sequence Synthetic peptide
EPKSCDKTYTCPPCP

SEQ ID NO: 110 - protein, 45 aa, Artificial Sequence
  Description of Artificial Sequence Synthetic polypeptide
TTTPAPRPPTPAPTIASQPLSLRPEACRPAAGGAVHTRGLDFACD

SEQ ID NO: 111 - protein, 10 aa, Artificial Sequence
  Description of Artificial Sequence Synthetic peptide
KSCDKTHTCP

SEQ ID NO: 112 - protein, 119 aa, Artificial Sequence
  Description of Artificial Sequence Synthetic polypeptide
QVQLQESGGGLVQAGGSLRLSCAASGFIFRAASMAWYRQSPGNERELVASISSGAFTNYADSVKARFTISRDNAKNTVYLQMNSLKPEDTAVYFCGATFLRSDGHHTINGQGTQVTVSS

SEQ ID NO: 113 - protein, 119 aa, Artificial Sequence
  Description of Artificial Sequence Synthetic polypeptide
QVQLQESGGGLVQAGGSLRLSCAASGFIFRAASMGWFRQSPGNERELVATVSSGDFTNYADSVKGRFTISRDNAKNTVYLQMNSLKPEDTAVYFCGATFVRSDGHHTIYGQGTQVTVSS

SEQ ID NO: 114 - protein, 119 aa, Artificial Sequence
  Description of Artificial Sequence Synthetic polypeptide
QVQLQESGGGLVQAGGSLRLSCAASGFIFRAASMDWYRQFPGNERESIATISSGGFTNYADSVKGRFTISRDNAKNTVYLQMNSLKPEDTAVYFCGATFLRSDGHHTINGQGTQVTVSS

SEQ ID NO: 115 - protein, 119 aa, Artificial Sequence
  Description of Artificial Sequence Synthetic polypeptide
QVQLQESGGGLVQAGGSLRLSCAASGFIFRAASMGWFRQSPGNERELVATVSSGGFTNYADSVKGRFTISRDNAKNTVYLQMNSLKPEDTAVYFCGATFVRSDGHHTIYGQGTQVTVSS

SEQ ID NO: 116 - protein, 119 aa, Artificial Sequence
  Description of Artificial Sequence Synthetic polypeptide
QVQLQESGGGLVQAGGSLRLSCAASGFIFRAASMDWYRQSPGTQPELVATISSTGFTNYANSVKGRFTISRDNAKNTVYLQMNSLKPEDTAVYFCGATFLRSDGQHSIYGQGTQVTVSS

SEQ ID NO: 117 - protein, 124 aa, Artificial Sequence
  Description of Artificial Sequence Synthetic polypeptide
QVQLQESGGGLVHTGGSLRLSCAASGDTFLRYAMGWFRQAPGKEREFVAAITWNGGNTDYAGSLKGRFTISRDNTKNTVYLQMNSLKPEDTAVYYCAADLTFGLASSHYQYDYWGQGTQVTVSS

SEQ ID NO: 118 - protein, 124 aa, Artificial Sequence
  Description of Artificial Sequence Synthetic polypeptide
QVQLQESGGGLVQAGGSLRLSCAASGDTFLRYAMGWFRQAPGKEREFVAAITWNGGNTDYAGSLKGRFTISRDNTKNTVYLQMNSLKPEDTAVYYCAADLTFGLASSHYQYDYWGQGTQVTVSS

SEQ ID NO: 119 - protein, 124 aa, Artificial Sequence
  Description of Artificial Sequence Synthetic polypeptide
QVQLQESGGGLVHTGGSLRLSCAFSGDTFLRYAMGWFRQAPGKEREFVAAITWNGGNTDYADSLKGRFTISRDNTKNTVYLQMNSLRPEDTAVYYCAADLTFGLASSHYQYDYWGQGTQVTVSS

SEQ ID NO: 120 - protein, 124 aa, Artificial Sequence
  Description of Artificial Sequence Synthetic polypeptide
QVQLQESGGGLVQPGGSLRLSCAASGDTFLRYAMGWFRQAPGKEREFVAAITWNGGNTDYAGSLKGRFTISRDNTKNTVYLQMNSLKPEDTAVYYCAADLTFGLASSHYQYDYWGQGTQVTVSS

SEQ ID NO: 121 - protein, 112 aa, Artificial Sequence
  Description of Artificial Sequence Synthetic polypeptide
QVQLQESGGGSVLAGGSLRLSCAASGFTFSSYYMSWVRQAPGKGLEWVSGIHYTGDWTNYADSVKGRFTISRDNAKNELYLEMNNLKPEDTAVYYCARGSDKGQGTQVTVSS

SEQ ID NO: 122 - protein, 112 aa, Artificial Sequence
  Description of Artificial Sequence Synthetic polypeptide
QVQLQESGGGSVQAGGSLRLSCAASGFTFSSYYMSWVRQAPGKGLEWVSGIHYTGDWTNYADSVKGRFTISRDNAKNELYLEMNNLKPEDTAVYYCARGSDKGQGTQVTVSS

SEQ ID NO: 123 - protein, 112 aa, Artificial Sequence
  Description of Artificial Sequence Synthetic polypeptide
QVQLQESGGGLVQPGGSLRLSCAASGFTFSDWAMSWVRQAPGKGLEWVSGIHYGDHTTHYADFVKGRFTISRDDAKNTLYLQMNSLKPEDTAVYYCARGSTKGQGTQVTVSS

SEQ ID NO: 124 - protein, 112 aa, Artificial Sequence
  Description of Artificial Sequence Synthetic polypeptide
QVQLQESGGGLVQPGGSLRLSCAASGFTFSDWAMSWVRQAPGKGLEWVSSIHYGDHTTHYADFVKGRFTISRDDAKNTLYLQMNSLKPEDTAVYYCEKGTTRGQGTQVTVSS

SEQ ID NO: 125 - protein, 119 aa, Artificial Sequence
  Description of Artificial Sequence Synthetic polypeptide
QVQLQESGGGLVQAGGSLKLSCAASGNVFRAATMAWYRQAPEKQREMVATIASGGTTNYADFVKGRFTISRDNAKNTVYLQMNTLKPEDTAVYYCNAGYLTSLGPKNYWGQGTQVTVSS

SEQ ID NO: 126 - protein, 119 aa, Artificial Sequence
  Description of Artificial Sequence Synthetic polypeptide
  MOD_RES (44)..(44): Any amino acid
QVQLQESGGGLVQPGGSLRLSCAASGNVFRAATMAWYRQAPEKXREMVATIASGGTTNYADFVKGRFTISRDNAKNTVYLQMNTLKPEDTAVYYCNAGYLTSLGPKNYWGQGTQVTVSS

SEQ ID NO: 127 - protein, 119 aa, Artificial Sequence
  Description of Artificial Sequence Synthetic polypeptide
QVQLQESGGGLVQAGGSLRLSCAASGNVFRAATMAWYRQVPEKQREMVATIASGGTTNYADFVKGRFTISRDNAKNTVYLQMNTLKPEDTAVYYCNALYLTSLGPKSYWGQGTQVTVSS

SEQ ID NO: 128 - protein, 111 aa, Artificial Sequence
  Description of Artificial Sequence Synthetic polypeptide
QVQLQESGGGLVQPGGSLRLSCAASGFAFGNHWMYWYRQAPGRGRELVASISSGGSTNYVDSVKGRFTISRDNARNTVYLQMYSLKPEDTAVYYCGTSDNWGQGTQVTVSS

SEQ ID NO: 129 - protein, 111 aa, Artificial Sequence
  Description of Artificial Sequence Synthetic polypeptide
QVQLQESGGGLVQAGGSLRLSCAASGFAFGNHWMYWYRQAPGRGRELVASISSGGSTNYVDSVKGRFTISRDNARNTVYLQMYSLRPEDTAVYYCGTSDNWGQGTQVTVSS

SEQ ID NO: 130 - protein, 111 aa, Artificial Sequence
  Description of Artificial Sequence Synthetic polypeptide
QVQLQESGGGLVQAGGSLRLSCAASGFAFGNHWMYWYRQAPGRGRELVASISSGGSTNYVDSVKGRFTISRDNARNTVYLQMYSLKPEDTAVYYCGTSDNWGQGTQVTVSS

SEQ ID NO: 131 - protein, 119 aa, Artificial Sequence
  Description of Artificial Sequence Synthetic polypeptide
QVQLQESGGGLVQAGGSLRLSCAASGFIFRAASISWYRQSPGNERELVATINSGGFTNYADSVLGRFTISRDNAKNTGYLQMNSLKPEDTAVYFCAATFLRSDGQPPIWGQGTQVTVSS

SEQ ID NO: 132 - protein, 119 aa, Artificial Sequence
  Description of Artificial Sequence Synthetic polypeptide
QVQLQESGGGLVQAGGSLRLSCAASGFIFRAASMGWYRQSPGNERELVATINSGGFTNYADSVKGRFTISRDNAKNTGYLQMNSLKPEDTAVYFCAATFLRSDGQPPIWGQGTQVTVSS

SEQ ID NO: 133 - protein, 122 aa, Artificial Sequence
  Description of Artificial Sequence Synthetic polypeptide
QVQLQESGGGLVQAGGSLRLSCAASEYILSMYRMAWYRQAPGKVRELVADMSSGGTTNYADFVKGRFTISRDNDRNTVYLQMNRLQPEDTAAYYCNVAGRTGPPSYDAFNNWGQGTQVTVSS

SEQ ID NO: 134 - protein, 122 aa, Artificial Sequence
  Description of Artificial Sequence Synthetic polypeptide
QVQLQESGGGLVQPGASLRVSCAASEYILSMYRMAWYRQAPGKVRELVADMSSGGTTNYADFVKGRFTISRDNDRNTVYLQMNRLQPEDTAAYYCNVAGRTGPPSYDAFNNWGQGTQVTVSS

SEQ ID NO: 135 - protein, 122 aa, Artificial Sequence
  Description of Artificial Sequence Synthetic polypeptide
QVQLQESGGGLVQPGGSLRLSCAASESISSFIAVGWYRQAPGKERELVAGINRSGFTYYTDSVKGRFSISRDNAKNTVLLQMTSLKPEDTAVYYCNAGGLYFSNAYTQGDYWGQGTQVTVSS

SEQ ID NO: 136 - protein, 122 aa, Artificial Sequence
  Description of Artificial Sequence Synthetic polypeptide
QVQLQESGGGLVQTGGSLRLSCAASESISSFIAVGWYRQAPGKERELVAGINRSGFTYYTDSVKGRFSISRDNAKNTVLLQMTSLKPEDTAVYYCNAGGLYFSNAYTQGDYWGQGTQVTVSS

SEQ ID NO: 137 - protein, 119 aa, Artificial Sequence
  Description of Artificial Sequence Synthetic polypeptide
QVQLQESGGGLVQAGGSLRLSCAASGSVFRANVMGWYRQAPGKQHELVARIDPGGTTTYADPVKGRFTISRDNAKKTVYLQMNSLKPDDTAVYYCNAIILLSGGPKDYWGQGTQVTVSS

SEQ ID NO: 138 - protein, 120 aa, Artificial Sequence
  Description of Artificial Sequence Synthetic polypeptide
QVQLQESGGGLVQAGGSLRLSCAPSGRTSSIFGMGWFRQAPGKEREFVASINWSGGSTSYADSVKGRFTISRDNAKNEMYLQMNSLKFEDTAVYVCAAAVLTNKPSWNFWGQGTQVTVSS

SEQ ID NO: 139 - protein, 121 aa, Artificial Sequence
  Description of Artificial Sequence Synthetic polypeptide
QVQLQESGGGLVQAGGSLRLSCAASGPIFSDTIRTMGWYRQAAGKQRELVATIASFPSRTNYVDSVKGRFTISRDIAKNTVYLQMDSLKPEDTAVYYCNVDLASIPTKTYWGQGTQVTVSS

SEQ ID NO: 140 - protein, 120 aa, Artificial Sequence
  Description of Artificial Sequence Synthetic polypeptide
QVQLQESGGGLVQAGGSLRLSCAASGSIFGINAMGWYRQAPGKQRESVAFITIGGNTNYLDSVKGRFTISRDNAKNTVYLQMNGLKPEDTAVYYCNTNPPLILTAGGLYWGQGTQVTVSS

SEQ ID NO: 141 - protein, 121 aa, Artificial Sequence
  Description of Artificial Sequence Synthetic polypeptide
QVQLQESGGGLVQPGGSLRLSCATSANRFNINVMGWYRQAPGQQRELVATINIGGSTDYADSVKGRFTISRDNAKNTVYLQLSDLKPEDTAVYYCNVKLRVSGPTGPNVYWGQGTQVTVSS

SEQ ID NO: 142 - protein, 116 aa, Artificial Sequence
  Description of Artificial Sequence Synthetic polypeptide
QVQLQESGGGLVQAGGSLKLSCTASGTILSTMAWYRQAPGKQRELVATISRGGTTNYSDSVKGRFAISRDSTKNTVYLQMNSLKPEDTAVYYCNTPLTDYGMGYNWGQGTQVTVSS

SEQ ID NO: 143 - protein, 120 aa, Artificial Sequence
  Description of Artificial Sequence Synthetic polypeptide
QVQLQESGGGLVQAGGSLRLSCAVSGSIFSLNTLAWYRQAPGRQRDLIARITGGGTTVYADSVKGRFTISRDNAKNTVYLQMNSLKPEDTAVYYCNLMVRHPSGSTYEYWGQGTQVTVSS

SEQ ID NO: 144 - protein, 119 aa, Artificial Sequence
  Description of Artificial Sequence Synthetic polypeptide
QVQLQESGGGLVQAGGSLRLSCAASGIIFRGTTMGWFRQAPGKQRESVASISPLGTTSYSGSVEGRFTVSRDNAKNTLFLQMNSLKSEDTAVYYCNAIQVTNVGPRVYWGQGTQVTVSS

SEQ ID NO: 145 - protein, 124 aa, Artificial Sequence
  Description of Artificial Sequence Synthetic polypeptide
QVQLQESGGGLVQPGGSLRLSCASSGFTLDDYTIGWFRQAPGKEREGVSCISRRDDSTYYADSVKGRFTISRDNAKNTVDLQMISLRPEDTAVYYCAATPRSYTLRCLGKFDFQGQGTQVTVSS

SEQ ID NO: 146 - protein, 121 aa, Artificial Sequence
  Description of Artificial Sequence Synthetic polypeptide
QVQLQESGGGLVQAGGSLRLSCAASGNIVRMTNMAWYRQAPGKQREFVATISAGGSTTYVDSVKDRFTISRDNTKNTVYLQMNYLKPEDTAVYYCATGSILTNRGAIPGSWGHGTQVTVSS

SEQ ID NO: 147 - protein, 127 aa, Artificial Sequence
  Description of Artificial Sequence Synthetic polypeptide
QVQLQESGGGLVQAGGSLRLSCAAPGFAFNDHAILWFRQAPGKEREGVSEICRDGTTYYTDSVKGRFTISSDNAKNTVYLQMNSVKTDDTAVYYCAVDRRRYYCSGNRAFSSDYYYWGQGTQVTVSS

SEQ ID NO: 148 - protein, 119 aa, Artificial Sequence
  Description of Artificial Sequence Synthetic polypeptide
QVQLQESGGGLVQAGGSLRLSCVHSGSIFRASTMAWYRQAPGKQRELVAQIMSGGGTNYAGSVKGRFTISRDNANNTVYLQMNSLKPEDTAVYYCNAAQITSWGPKVYWGQGTQVTVSS

SEQ ID NO: 149 - protein, 122 aa, Artificial Sequence
  Description of Artificial Sequence Synthetic polypeptide
QVQLQESGGGLVQPGGSLRLSCAASGRINSINTMGWYRQAPGNQRELVAEITRGGTTNYADSVQGRYAISRDNAKNLVYLQMNSLKPEDTDVYYCNAQTFPTFSRPTGLDYWGQGTQVTVSS

SEQ ID NO: 150 - protein, 10 aa, Artificial Sequence
  Description of Artificial Sequence Synthetic peptide
GFIFRAASMA

SEQ ID NO: 151 - protein, 10 aa, Artificial Sequence
  Description of Artificial Sequence Synthetic peptide
GFIFRAASMG

SEQ ID NO: 152 - protein, 10 aa, Artificial Sequence
  Description of Artificial Sequence Synthetic peptide
GFIFRAASMD

SEQ ID NO: 153 - protein, 10 aa, Artificial Sequence
  Description of Artificial Sequence Synthetic peptide
GFIFRAASMG

SEQ ID NO: 154 - protein, 10 aa, Artificial Sequence
  Description of Artificial Sequence Synthetic peptide
GFIFRAASMD

SEQ ID NO: 155 - protein, 10 aa, Artificial Sequence
  Description of Artificial Sequence Synthetic peptide
GDTFLRYAMG

SEQ ID NO: 156 - protein, 10 aa, Artificial Sequence
  Description of Artificial Sequence Synthetic peptide
GDTFLRYAMG

SEQ ID NO: 157 - protein, 10 aa, Artificial Sequence
  Description of Artificial Sequence Synthetic peptide
GDTFLRYAMG

SEQ ID NO: 158 - protein, 10 aa, Artificial Sequence
  Description of Artificial Sequence Synthetic peptide
GDTFLRYAMG

SEQ ID NO: 159 - protein, 10 aa, Artificial Sequence
  Description of Artificial Sequence Synthetic peptide
GFTFSSYYMS

SEQ ID NO: 160 - protein, 10 aa, Artificial Sequence
  Description of Artificial Sequence Synthetic peptide
GFTFSDWAMS

SEQ ID NO: 161 - protein, 10 aa, Artificial Sequence
  Description of Artificial Sequence Synthetic peptide
GFTFSDWAMS

SEQ ID NO: 162 - protein, 10 aa, Artificial Sequence
  Description of Artificial Sequence Synthetic peptide
GNVFRAATMA

SEQ ID NO: 163 - protein, 10 aa, Artificial Sequence
  Description of Artificial Sequence Synthetic peptide
GNVFRAATMA

SEQ ID NO: 164 - protein, 10 aa, Artificial Sequence
  Description of Artificial Sequence Synthetic peptide
GNVFRAATMA

SEQ ID NO: 165 - protein, 10 aa, Artificial Sequence
  Description of Artificial Sequence Synthetic peptide
GFAFGNHWMY

SEQ ID NO: 166 - protein, 10 aa, Artificial Sequence
  Description of Artificial Sequence Synthetic peptide
GFAFGNHWMY

SEQ ID NO: 167 - protein, 10 aa, Artificial Sequence
  Description of Artificial Sequence Synthetic peptide
GFAFGNHWMY

SEQ ID NO: 168 - protein, 10 aa, Artificial Sequence
  Description of Artificial Sequence Synthetic peptide
GFIFRAASIS

SEQ ID NO: 169 - protein, 10 aa, Artificial Sequence
  Description of Artificial Sequence Synthetic peptide
GFIFRAASMG

SEQ ID NO: 170 - protein, 10 aa, Artificial Sequence
  Description of Artificial Sequence Synthetic peptide
EYILSMYRMA

SEQ ID NO: 171 - protein, 10 aa, Artificial Sequence
  Description of Artificial Sequence Synthetic peptide
EYILSMYRMA

SEQ ID NO: 172 - protein, 10 aa, Artificial Sequence
  Description of Artificial Sequence Synthetic peptide
ESISSFIAVG

SEQ ID NO: 173 - protein, 10 aa, Artificial Sequence
  Description of Artificial Sequence Synthetic peptide
ESISSFIAVG

SEQ ID NO: 174 - protein, 10 aa, Artificial Sequence
  Description of Artificial Sequence Synthetic peptide
GSVFRANVMG

SEQ ID NO: 175 - protein, 10 aa, Artificial Sequence
  Description of Artificial Sequence Synthetic peptide
GRTSSIFGMG

SEQ ID NO: 176 - protein, 12 aa, Artificial Sequence
  Description of Artificial Sequence Synthetic peptide
GPIFSDTIRTMG

SEQ ID NO: 177 - protein, 10 aa, Artificial Sequence
  Description of Artificial Sequence Synthetic peptide
GSIFGINAMG

SEQ ID NO: 178 - protein, 10 aa, Artificial Sequence
  Description of Artificial Sequence Synthetic peptide
ANRFNINVMG

SEQ ID NO: 179 - protein, 8 aa, Artificial Sequence
  Description of Artificial Sequence Synthetic peptide
GTILSTMA

SEQ ID NO: 180 - protein, 10 aa, Artificial Sequence
  Description of Artificial Sequence Synthetic peptide
GSIFSLNTLA

SEQ ID NO: 181 - protein, 10 aa, Artificial Sequence
  Description of Artificial Sequence Synthetic peptide
GIIFRGTTMG

SEQ ID NO: 182 - protein, 10 aa, Artificial Sequence
  Description of Artificial Sequence Synthetic peptide
GFTLDDYTIG

SEQ ID NO: 183 - protein, 10 aa, Artificial Sequence
  Description of Artificial Sequence Synthetic peptide
GNIVRMTNMA

SEQ ID NO: 184 - protein, 10 aa, Artificial Sequence
  Description of Artificial Sequence Synthetic peptide
GFAFNDHAIL

SEQ ID NO: 185 - protein, 10 aa, Artificial Sequence
  Description of Artificial Sequence Synthetic peptide
GSIFRASTMA

SEQ ID NO: 186 - protein, 10 aa, Artificial Sequence
  Description of Artificial Sequence Synthetic peptide
GRINSINTMG

SEQ ID NO: 187 - protein, 16 aa, Artificial Sequence
  Description of Artificial Sequence Synthetic peptide
SISSGAFTNYADSVKA

SEQ ID NO: 188 - protein, 16 aa, Artificial Sequence
  Description of Artificial Sequence Synthetic peptide
TVSSGDFTNYADSVKG

SEQ ID NO: 189 - protein, 16 aa, Artificial Sequence
  Description of Artificial Sequence Synthetic peptide
TISSGGFTNYADSVKG

SEQ ID NO: 190 - protein, 16 aa, Artificial Sequence
  Description of Artificial Sequence Synthetic peptide
TVSSGGFTNYADSVKG

SEQ ID NO: 191 - protein, 16 aa, Artificial Sequence
  Description of Artificial Sequence Synthetic peptide
TISSTGFTNYANSVKG

SEQ ID NO: 192 - protein, 17 aa, Artificial Sequence
  Description of Artificial Sequence Synthetic peptide
AITWNGGNTDYAGSLKG

SEQ ID NO: 193 - protein, 17 aa, Artificial Sequence
  Description of Artificial Sequence Synthetic peptide
AITWNGGNTDYAGSLKG

SEQ ID NO: 194 - protein, 17 aa, Artificial Sequence
  Description of Artificial Sequence Synthetic peptide
AITWNGGNTDYADSLKG

SEQ ID NO: 195 - protein, 17 aa, Artificial Sequence
  Description of Artificial Sequence Synthetic peptide
AITWNGGNTDYAGSLKG

SEQ ID NO: 196 - protein, 17 aa, Artificial Sequence
  Description of Artificial Sequence Synthetic peptide
GIHYTGDWTNYADSVKG

SEQ ID NO: 197 - protein, 17 aa, Artificial Sequence
  Description of Artificial Sequence Synthetic peptide
GIHYTGDWTNYADSVKG

SEQ ID NO: 198 - protein, 17 aa, Artificial Sequence
  Description of Artificial Sequence Synthetic peptide
GIHYGDHTTHYADFVKG

SEQ ID NO: 199 - protein, 17 aa, Artificial Sequence
  Description of Artificial Sequence Synthetic peptide
SIHYGDHTTHYADFVKG

SEQ ID NO: 200 - protein, 16 aa, Artificial Sequence
  Description of Artificial Sequence Synthetic peptide
TIASGGTTNYADFVKG

SEQ ID NO: 201 - protein, 16 aa, Artificial Sequence
  Description of Artificial Sequence Synthetic peptide
TIASGGTTNYADFVKG

SEQ ID NO: 202 - protein, 16 aa, Artificial Sequence
  Description of Artificial Sequence Synthetic peptide
TIASGGTTNYADFVKG

SEQ ID NO: 203 - protein, 16 aa, Artificial Sequence
  Description of Artificial Sequence Synthetic peptide
SISSGGSTNYVDSVKG

SEQ ID NO: 204 - protein, 16 aa, Artificial Sequence
  Description of Artificial Sequence Synthetic peptide
SISSGGSTNYVDSVKG

SEQ ID NO: 205 - protein, 16 aa, Artificial Sequence
  Description of Artificial Sequence Synthetic peptide
SISSGGSTNYVDSVKG

SEQ ID NO: 206 - protein, 16 aa, Artificial Sequence
  Description of Artificial Sequence Synthetic peptide
TINSGGFTNYADSVLG

SEQ ID NO: 207 - protein, 16 aa, Artificial Sequence
  Description of Artificial Sequence Synthetic peptide
TINSGGFTNYADSVKG

SEQ ID NO: 208 - protein, 16 aa, Artificial Sequence
  Description of Artificial Sequence Synthetic peptide
DMSSGGTTNYADFVKG

SEQ ID NO: 209 - protein, 16 aa, Artificial Sequence
  Description of Artificial Sequence Synthetic peptide
DMSSGGTTNYADFVKG

SEQ ID NO: 210 - protein, 16 aa, Artificial Sequence
  Description of Artificial Sequence Synthetic peptide
GINRSGFTYYTDSVKG

SEQ ID NO: 211 - protein, 16 aa, Artificial Sequence
  Description of Artificial Sequence Synthetic peptide
GINRSGFTYYTDSVKG

SEQ ID NO: 212 - protein, 16 aa, Artificial Sequence
  Description of Artificial Sequence Synthetic peptide
RIDPGGTTTYADPVKG

SEQ ID NO: 213 - protein, 17 aa, Artificial Sequence
  Description of Artificial Sequence Synthetic peptide
SINWSGGSTSYADSVKG

SEQ ID NO: 214 - protein, 17 aa, Artificial Sequence
  Description of Artificial Sequence Synthetic peptide
TIASFPSRTNYVDSVKG

SEQ ID NO: 215 - protein, 16 aa, Artificial Sequence
  Description of Artificial Sequence Synthetic peptide
FITIGGNTNYLDSVKG

SEQ ID NO: 216 - protein, 16 aa, Artificial Sequence
  Description of Artificial Sequence Synthetic peptide
TINIGGSTDYADSVKG

SEQ ID NO: 217 - protein, 16 aa, Artificial Sequence
  Description of Artificial Sequence Synthetic peptide
TISRGGTTNYSDSVKG

SEQ ID NO: 218 - protein, 16 aa, Artificial Sequence
  Description of Artificial Sequence Synthetic peptide
RITGGGTTVYADSVKG

SEQ ID NO: 219 - protein, 16 aa, Artificial Sequence
  Description of Artificial Sequence Synthetic peptide
SISPLGTTSYSGSVEG

SEQ ID NO: 220 - protein, 17 aa, Artificial Sequence
  Description of Artificial Sequence Synthetic peptide
CISRRDDSTYYADSVKG

SEQ ID NO: 221 - protein, 16 aa, Artificial Sequence
  Description of Artificial Sequence Synthetic peptide
TISAGGSTTYVDSVKD

SEQ ID NO: 222 - protein, 16 aa, Artificial Sequence
  Description of Artificial Sequence Synthetic peptide
EICRDGTTYYTDSVKG

SEQ ID NO: 223 - protein, 16 aa, Artificial Sequence
  Description of Artificial Sequence Synthetic peptide
QIMSGGGTNYAGSVKG

SEQ ID NO: 224 - protein, 16 aa, Artificial Sequence
  Description of Artificial Sequence Synthetic peptide
EITRGGTTNYADSVQG

SEQ ID NO: 225 - protein, 11 aa, Artificial Sequence
  Description of Artificial Sequence Synthetic peptide
TFLRSDGHHTI

SEQ ID NO: 226 - protein, 11 aa, Artificial Sequence
  Description of Artificial Sequence Synthetic peptide
TFVRSDGHHTI

SEQ ID NO: 227 - protein, 11 aa, Artificial Sequence
  Description of Artificial Sequence Synthetic peptide
TFLRSDGHHTI

SEQ ID NO: 228 - protein, 11 aa, Artificial Sequence
  Description of Artificial Sequence Synthetic peptide
TFVRSDGHHTI

SEQ ID NO: 229 - protein, 11 aa, Artificial Sequence
  Description of Artificial Sequence Synthetic peptide
TFLRSDGQHSI

SEQ ID NO: 230 - protein, 15 aa, Artificial Sequence
  Description of Artificial Sequence Synthetic peptide
DLTFGLASSHYQYDY

SEQ ID NO: 231 - protein, 15 aa, Artificial Sequence
  Description of Artificial Sequence Synthetic peptide
DLTFGLASSHYQYDY

SEQ ID NO: 232 - protein, 15 aa, Artificial Sequence
  Description of Artificial Sequence Synthetic peptide
DLTFGLASSHYQYDY

SEQ ID NO: 233 - protein, 15 aa, Artificial Sequence
  Description of Artificial Sequence Synthetic peptide
DLTFGLASSHYQYDY

SEQ ID NO: 234 - protein, 3 aa, Artificial Sequence
  Description of Artificial Sequence Synthetic peptide
GSD

SEQ ID NO: 235 - protein, 3 aa, Artificial Sequence
  Description of Artificial Sequence Synthetic peptide
GSD

SEQ ID NO: 236 - protein, 3 aa, Artificial Sequence
  Description of Artificial Sequence Synthetic peptide
GST

SEQ ID NO: 237 - protein, 3 aa, Artificial Sequence
  Description of Artificial Sequence Synthetic peptide
GTT

SEQ ID NO: 238 - protein, 11 aa, Artificial Sequence
  Description of Artificial Sequence Synthetic peptide
GYLTSLGPKNY

SEQ ID NO: 239 - protein, 11 aa, Artificial Sequence
  Description of Artificial Sequence Synthetic peptide
GYLTSLGPKNY

SEQ ID NO: 240 - protein, 11 aa, Artificial Sequence
  Description of Artificial Sequence Synthetic peptide
LYLTSLGPKSY

SEQ ID NO: 241 - protein, 3 aa, Artificial Sequence
  Description of Artificial Sequence Synthetic peptide
SDN

SEQ ID NO: 242 - protein, 3 aa, Artificial Sequence
  Description of Artificial Sequence Synthetic peptide
SDN

SEQ ID NO: 243 - protein, 3 aa, Artificial Sequence
  Description of Artificial Sequence Synthetic peptide
SDN

SEQ ID NO: 244 - protein, 11 aa, Artificial Sequence
  Description of Artificial Sequence Synthetic peptide
TFLRSDGQPPI

SEQ ID NO: 245 - protein, 11 aa, Artificial Sequence
  Description of Artificial Sequence Synthetic peptide
TFLRSDGQPPI

SEQ ID NO: 246 - protein, 14 aa, Artificial Sequence
  Description of Artificial Sequence Synthetic peptide
AGRTGPPSYDAFNN

SEQ ID NO: 247 - protein, 14 aa, Artificial Sequence
  Description of Artificial Sequence Synthetic peptide
AGRTGPPSYDAFNN

SEQ ID NO: 248 - protein, 14 aa, Artificial Sequence
  Description of Artificial Sequence Synthetic peptide
GGLYFSNAYTQGDY

SEQ ID NO: 249 - protein, 14 aa, Artificial Sequence
  Description of Artificial Sequence Synthetic peptide
GGLYFSNAYTQGDY

SEQ ID NO: 250 - protein, 11 aa, Artificial Sequence
  Description of Artificial Sequence Synthetic peptide
IILLSGGPKDY

SEQ ID NO: 251 - protein, 11 aa, Artificial Sequence
  Description of Artificial Sequence Synthetic peptide
AVLTNKPSWNF

SEQ ID NO: 252 - protein, 10 aa, Artificial Sequence
  Description of Artificial Sequence Synthetic peptide
DLASIPTKTY

SEQ ID NO: 253 - protein, 12 aa, Artificial Sequence
  Description of Artificial Sequence Synthetic peptide
NPPLILTAGGLY

SEQ ID NO: 254 - protein, 13 aa, Artificial Sequence
  Description of Artificial Sequence Synthetic peptide
KLRVSGPTGPNVY

SEQ ID NO: 255 - protein, 10 aa, Artificial Sequence
  Description of Artificial Sequence Synthetic peptide
PLTDYGMGYN

SEQ ID NO: 256 - protein, 12 aa, Artificial Sequence
  Description of Artificial Sequence Synthetic peptide
MVRHPSGSTYEY

SEQ ID NO: 257 - protein, 11 aa, Artificial Sequence
  Description of Artificial Sequence Synthetic peptide
IQVTNVGPRVY

SEQ ID NO: 258 - protein, 15 aa, Artificial Sequence
  Description of Artificial Sequence Synthetic peptide
TPRSYTLRCLGKFDF

SEQ ID NO: 259 - protein, 13 aa, Artificial Sequence
  Description of Artificial Sequence Synthetic peptide
GSILTNRGAIPGS

SEQ ID NO: 260 - protein, 19 aa, Artificial Sequence
  Description of Artificial Sequence Synthetic peptide
DRRRYYCSGNRAFSSDYYY

SEQ ID NO: 261 - protein, 11 aa, Artificial Sequence
  Description of Artificial Sequence Synthetic peptide
AQITSWGPKVY

SEQ ID NO: 262 - protein, 14 aa, Artificial Sequence
  Description of Artificial Sequence Synthetic peptide
QTFPTFSRPTGLDY

SEQ ID NO: 263 - protein, 111 aa, Artificial Sequence
  Description of Artificial Sequence Synthetic polypeptide
EVQLVESGGGLVQPGGSLTLSCAASGFAFGNHWMYWYRQAPGRGRELVASISSGGSTNYVDSVKGRFTISRDNAKNTLYLQMNSLRAEDTAVYYCGTSDNWGQGTLVTVSS

SEQ ID NO: 264 - protein, 119 aa, Artificial Sequence
  Description of Artificial Sequence Synthetic polypeptide
EVQLLESGGGLVQPGGSLTLSCAASGFIFRAASMDWYRQFPGNERESIATISSGGFTNYADSVKGRFTISRDNSKNTLYLQMNSLRAEDTAVYYCGATFLRSDGHHTINGQGTLVTVSS

SEQ ID NO: 265 - protein, 10 aa, Artificial Sequence
  Description of Artificial Sequence Synthetic peptide
KPLGLQARVV

SEQ ID NO: 266 - protein, 10 aa, Artificial Sequence
  Description of Artificial Sequence Synthetic peptide
PQASTGRSGG

SEQ ID NO: 267 - protein, 10 aa, Artificial Sequence
  Description of Artificial Sequence Synthetic peptide
PQGSTGRAAG

SEQ ID NO: 268 - protein, 10 aa, Artificial Sequence
  Description of Artificial Sequence Synthetic peptide
PPASSGRAGG

SEQ ID NO: 269 - protein, 9 aa, Artificial Sequence
  Description of Artificial Sequence Synthetic peptide
PIPVQGRAH

SEQ ID NO: 270 - protein, 10 aa, Artificial Sequence
  Description of Artificial Sequence Synthetic peptide
SGGGGSGGVV

SEQ ID NO: 271 - protein, 16 aa, Artificial Sequence
  Description of Artificial Sequence Synthetic peptide
SGGGGSGGGGSGGGGS

SEQ ID NO: 272 - protein, 17 aa, Artificial Sequence
  Description of Artificial Sequence Synthetic peptide
SGGGGSGGGGSGGGGGS

SEQ ID NO: 273 - protein, 10 aa, Artificial Sequence
  Description of Artificial Sequence Synthetic peptide
SGGGGSGGGS

SEQ ID NO: 274 - protein, 10 aa, Artificial Sequence
  Description of Artificial Sequence Synthetic peptide
KPLGLQARVV

SEQ ID NO: 275 - protein, 10 aa, Artificial Sequence
  Description of Artificial Sequence Synthetic peptide
PQASTGRSGG

SEQ ID NO: 276 - protein, 10 aa, Artificial Sequence
  Description of Artificial Sequence Synthetic peptide
PQGSTGRAAG

SEQ ID NO: 277 - protein, 10 aa, Artificial Sequence
  Description of Artificial Sequence Synthetic peptide
PPASSGRAGG

SEQ ID NO: 278 - protein, 9 aa, Artificial Sequence
  Description of Artificial Sequence Synthetic peptide
PIPVQGRAH

SEQ ID NO: 279 - protein, 10 aa, Artificial Sequence
  Description of Artificial Sequence Synthetic peptide
PQGSTARSAG

SEQ ID NO: 280 - protein, 5 aa, Artificial Sequence
  Description of Artificial Sequence Synthetic peptide
GSGGS

SEQ ID NO: 281 - protein, 4 aa, Artificial Sequence
  Description of Artificial Sequence Synthetic peptide
GGGS

SEQ ID NO: 282 - protein, 4 aa, Artificial Sequence
  Description of Artificial Sequence Synthetic peptide
GGSG

SEQ ID NO: 283 - protein, 5 aa, Artificial Sequence
  Description of Artificial Sequence Synthetic peptide
GGSGG

SEQ ID NO: 284 - protein, 5 aa, Artificial Sequence
  Description of Artificial Sequence Synthetic peptide
GSGSG

SEQ ID NO: 285 - protein, 5 aa, Artificial Sequence
  Description of Artificial Sequence Synthetic peptide
GSGGG

SEQ ID NO: 286 - protein, 5 aa, Artificial Sequence
  Description of Artificial Sequence Synthetic peptide
GGGSG

SEQ ID NO: 287 - protein, 5 aa, Artificial Sequence
  Description of Artificial Sequence Synthetic peptide
GSSSG

SEQ ID NO: 288 - protein, 5 aa, Artificial Sequence
  Description of Artificial Sequence Synthetic peptide
DKTHT

SEQ ID NO: 289 - protein, 4 aa, Artificial Sequence
  Description of Artificial Sequence Synthetic peptide
CPPC

SEQ ID NO: 290 - protein, 20 aa, Artificial Sequence
  Description of Artificial Sequence Synthetic peptide
  MISC_FEATURE (1)..(20): This sequence may encompass 1-4 “Gly Gly Gly Gly Ser” repeating units
GGGGSGGGGSGGGGSGGGGS

SEQ ID NO: 291 - protein, 20 aa, Artificial Sequence
  Description of Artificial Sequence Synthetic peptide
  MISC_FEATURE (1)..(20): This sequence may encompass 2-4 “Gly Gly Gly Gly Ser” repeating units
GGGGSGGGGSGGGGSGGGGS

SEQ ID NO: 292 - protein, 15 aa, Artificial Sequence
  Description of Artificial Sequence Synthetic peptide
  MISC_FEATURE (1)..(15): This sequence may encompass 1-3 “Gly Gly Gly Gly Ser” repeating units
GGGGSGGGGSGGGGS